(19) United States
(12) Patent Application Publication
(10) Pub. No.: US 2022/0218747 A1
(43) Pub. Date: Jul. 14, 2022

(54) METHODS FOR SELECTIVE IN VIVO EXPANSION OF GAMMA DELTA T-CELL POPULATIONS AND COMPOSITIONS THEREOF

(71) Applicant: ADICET BIO, INC., Menlo Park, CA (US)

(72) Inventors: Aya JAKOBOVITS, Menlo Park, CA (US); Daulet SATPAYEV, Menlo Park, CA (US); Orit FOORD, Menlo Park, CA (US); Yifeng Frank JING, Menlo Park, CA (US); Hui SHAO, Menlo Park, CA (US)

(21) Appl. No.: 17/298,903

(22) PCT Filed: Dec. 3, 2019
(86) PCT No.: PCT/US2019/064319

§ 371 Date: Jun. 1, 2021

Related U.S. Application Data
(60) Provisional application No. 62/774,817, filed on Dec. 3, 2018.

(51) Int. Cl. A61K 35/17; A61K 38/20; C07K 16/28

(52) U.S. Cl. CPC A61K 35/17; C07K 2317/70; C07K 16/2809; A61K 38/20

(57) ABSTRACT

The present invention relates to methods for the selective in vivo activation, expansion and/or maintenance of γδ T-cell population(s), compositions and admixtures thereof and methods for using the same as a therapeutic. Methods and compositions of the disclosure are useful in the treatment of various cancers, infectious diseases, and immune disorders.

### CROSS-REFERENCES TO RELATED APPLICATIONS

The present application claims the benefit of priority to U.S. Provisional Application No. 62/774,817, filed on Dec. 3, 2018, the contents of which are hereby incorporated by reference in the entirety and for all purposes.

### BACKGROUND

Antigen recognition by T lymphocytes may be achieved by highly diverse heterodimeric receptors, the T-cell receptors (TCRs). Approximately 95% of human T-cells in blood and lymphoid organs express a heterodimeric αβ TCR receptor αβ T-cell lineage). Approximately 5% of human T-cells in the blood and lymphoid organs express heterodimeric γδ TCR receptor (γδ T-cell lineage). These T-cell subsets may be referred to as ‘αβ’ and ‘γδ’ T-cells, respectively. αβ and γδ T-cells are different in function. Activation of αβ T-cells then occurs when an antigen presenting cell (APC) presents an antigen in the context of class I/II MHC. In contrast to αβ T-cells, γδ T-cells can recognize an antigen independent of MHC restriction. In addition, γδ T-cells combine both innate and adoptive immune recognition and responses.

γδ T cells utilize a distinct set of somatically rearranged variable (V), diversity (D), joining (J), and constant (C) genes. γδ T cells contain fewer V, D, and J segments than αβ T cells. Although the number of germline Vγ and Vδ genes is more limited than the repertoire of Vα and Vβ TCR genes, more extensive junctional diversification processes during TCR γ and δ chain rearrangement leads to a potential larger γδ TCRs repertoire than that of αβTCRs (Carding and Egan, Nat Rev Immunol (2002) 2:336).

Human γδ T-cells use 3 main Vδ (Vδ1, Vδ2, Vδ3) and at most six Vγ region genes to make their TCRs (Hayday A C., Annu Rev Immunol. 2000; 18, 975-1026). Two main Vδ subsets are Vδ1 and Vδ2 γδ T cells. Vδ1 T cells with different Vγ predominate in the intraepithelial subset of mucosal γδ T cells where the TCRs appear to recognize stress molecules on epithelial cells (Beagley K W, Husband A J. Crit Rev Immunol. 1998; 18(3):237-254). Vδ2 T cells that generally coexpress Vγ9 are abundant in the peripheral blood and lymphatic system.

The ability of γδ T-cells to recognize an antigen on diseased cells directly and to exhibit inherent ability to kill tumor cells renders γδ T-cells an attractive therapeutic tool. The abundant Vγ9Vδ2 sub-type of γδ T cells recognize pyrophosphate compounds, such as the microbial compound (E)-4-hydroxy-3-methyl-but-2-enyl pyrophosphate. However, the ligand recognized by other γδ T-cell sub-types is unknown.

Adoptive transfer of Vγ9Vδ2 T cells has yielded limited objective clinical responses for investigational treatment of cancer (Kondo et al, Cytotherapy, 10:842-856, 2008; Lang et al, Cancer Immunology, Immunotherapy: CII, 60: 1447-1460, 2011; Nagamine et al, 2009; Nicol et al, British Journal of Cancer, 105:778-786, 2011; Wilhelm et al, Blood. 2003 Jul. 1; 102(1):200-6), indicating the need to isolate and test clinically new γδ T-cell populations.

The ability to selectively expand γδ T-cell subset populations having potent anti-tumor activity with improved purity and in clinically-relevant levels is highly desirable. Although antibodies and cytokine cocktails have been used to propagate a more diverse set of γδ T cells, activation of specific γδ T-cell subsets to sufficient purity and clinically-relevant levels, was not achieved (Dokouhaki et al, 2010; Kang et al, 2009; Lopez et al, 2000; Kress, 2006).

Selective expansion of γδ T-cell sub-types has been demonstrated ex vivo and in vivo by the use of known ligands of Vγ9Vδ2. For example, Pressey et al., Medicine (Baltimore). 2016 September; 95(39): e4909, reports in vivo expansion of Vγ9Vδ2 using intravenous zoledronate, a synthetic pyrophosphate mimic, and subcutaneous IL-2. Selective expansion of other γδ T-cell sub-types has been demonstrated ex vivo using immobilized antibodies that selectively bind and cross-link, e.g., δ1, δ2, and δ3 sub-types. See, WO 2016/081518; WO 2017/197347; and WO 2019/099744, the contents of which are incorporated in the entirety. Unfortunately, however, in vivo immobilization is typically performed by binding the antibody to an Fc receptor on the surface of a cell, which would generally be expected to induce an antibody-dependent cell-mediated cytotoxicity (ADCC) effect and thereby reduce the population of γδ T-cells recognized by the antibody. Similarly, methods of reducing the interaction between Fc receptor and the antibody also reduce immobilization and therefore would also not be expected to achieve robust in vivo expansion. Accordingly, clinically-relevant methods of expanding specific γδ T cell subsets in vivo, and the cells produced thereby, are greatly needed.

### SUMMARY OF THE INVENTION

The present inventors have identified i) activating agents that selectively activate and expand δ1 T cells by binding to an activating epitope specific of a M TCR, ii) activating agents that selectively activate and expand δ2 T cells by binding to an activating epitope specific of a δ2 TCR; and activating agents that selectively activate and expand δ3 T cells by binding to an activating epitope specific of a δ3 TCR. The present inventors have surprisingly found that, in a physiologically relevant setting, such antibodies can effectively activate and expand, as well as support the persistence of, adoptively transferred chimeric antigen receptor (CAR) γδ T-cells and/or endogenous γδ T-cells in vivo.

Described herein, are methods and compositions for using these activating agents, individually or in combinations, for in vivo expansion of γδ T cells. These methods and compositions can be suitable for selective activation and expansion of one or more γδ T cell sub-populations. The in vivo expansion methods and compositions can be suitable for maintaining and/or expanding endogenous γδ T cells or a sub-population thereof. Additionally, or alternatively, the in vivo expansion methods and compositions can be suitable for maintaining and/or or expanding γδ T cells, or a sub-population thereof, that have been administered to a subject.

In a first aspect, the present invention provides an in vivo method for activating, expanding and/or maintaining a population of T cells in a subject, the method comprising administering to the subject an effective amount of one or more agents which selectively expand δ1 T cells, δ2 T cells, or δ3 T cells, or a combination thereof, wherein the one or more agents which selectively expand δ1 T cells bind to an activating epitope specific of a δ1 TCR; and the one or more agents which selectively expand δ2 T cells bind to an activating epitope specific of a δ2 TCR; the one or more agents which selectively expand δ3 T cells bind to an activating epitope specific of a δ3 TCR, thereby activating, expanding, or maintaining the population of γδ T cells in the subject.

In some embodiments, the method comprises administering to the subject an effective amount of one or more agents which selectively expand M T cells. In some embodiments, the agent that selectively expands δ1 T-cells is selected from an agent which binds to the same epitope as an antibody selected from TS-1 and TS8.2. In some embodiments, the agent that binds the same epitope as an antibody selected from TS-1 and TS8.2 comprises the CDRs of TS-1 or TS8.2 and/or is a humanized TS-1 or TS8.2. In some embodiments, the agent that selectively expands M T-cells is selected from an agent that does not compete with TS-1, TS8.2, or R9.12. In some embodiments, the agent that selectively expands δ1 T-cells is selected from an agent which specifically binds to an epitope comprising a M variable region. In some embodiments, the agent that selectively expands M T-cells binds to an epitope comprising residues Arg71, Asp72 and Lys120 of the M variable region. In some embodiments, the agent that selectively expands δ1 T-cells has reduced binding to a mutant M TCR polypeptide comprising a mutation at K120 of delta J1 and delta J2.

In some embodiments, the agents that selectively expand M T-cells are agents that bind a δ1 TCR Bin 1 M epitope, Bin 1b δ1 epitope, Bin 2 δ1 epitope, Bin 2b M epitope, Bin 2c δ1 epitope, Bin 3 M epitope, Bin 4 δ1 epitope, Bin 5 δ1 epitope, Bin 6 δ1 epitope, Bin 7 δ1 epitope, Bin 8 δ1 epitope, or a Bin 9 δ1 epitope of a human M TCR. In some embodiments, the agent that selectively expands M T-cells is selected from an agent which binds to the same, or essentially the same, epitope as, or competes with, an antibody selected from the group consisting of δ1-05, δ1-08, δ1-18, δ1-22, δ1-23, δ1-26, δ1-35, δ1-37, δ1-39, δ1-113, δ1-143, δ1-149, δ1-155, δ1-182, δ1-183, δ1-191, δ1-192, δ1-195, δ1-197, δ1-199, δ1-201, δ1-203, δ1-239, δ1-253, δ1-257, δ1-278, δ1-282, and δ1-285.

In some embodiments, the agent that selectively expands M T-cells is an antibody selected from the group consisting of δ1-05, δ1-08, δ1-18, δ1-22, δ1-23, δ1-26, δ1-35, δ1-37, δ1-39, δ1-113, δ1-143, δ1-149, δ1-155, δ1-182, δ1-183, δ1-191, δ1-192, δ1-195, δ1-197, δ1-199, δ1-201, δ1-203, δ1-239, δ1-253, δ1-257, δ1-278, δ1-282, and δ1-285. In some embodiments, the agent that selectively expands δ1 T-cells selectively expands δ1 T cells and δ3 T cells. In some embodiments, the agent that selectively expands δ1 T cells selectively expands δ1, δ3, δ4, and δ5 γδ T cells.

In some embodiments, the method comprises administering to the subject an effective amount of one or more agents which selectively expand δ2 T cells. In some embodiments, the agent that selectively expands δ2 T-cells is selected from an agent which binds to the same epitope as an antibody selected from 15D and B6. In some embodiments, the agent that binds the same epitope as an antibody selected from 15D and B6 comprises the CDRs of 15D or B6 and/or is a humanized 15D and B6. In some embodiments, the agent that selectively expands δ2 T-cells is selected from an agent which binds to a different epitope as antibody 15D and/or B6. In some embodiments, the agent that selectively expands δ2 T-cells is selected from an agent which specifically binds to an epitope comprising a δ2 variable region. In some embodiments, the agent that selectively expands δ2 T-cells has reduced binding to a mutant δ2 TCR polypeptide comprising a mutation at G35 of the δ2 variable region. In some embodiments, the agent that selectively expands δ2 T-cells binds a δ2 TCR Bin 1 δ2 epitope, Bin 2 δ2 epitope, Bin 3 δ2 epitope, or Bin 4 δ2 epitope of a human δ2 TCR.

In some embodiments, the agent that selectively expands δ2 T-cells is an agent that binds to the same, or essentially the same, epitope as, or competes with, an antibody selected from the group consisting of δ2-14, δ2-17, δ2-22, δ2-30, δ2-31, δ2-32, δ2-33, δ2-35, δ2-36, and δ2-37. In some embodiments, the agent that selectively expands δ2 T-cells is an antibody selected from the group consisting of δ2-14, δ2-17, δ2-22, δ2-30, δ2-31, δ2-32, δ2-33, δ2-35, δ2-36, and δ2-37.

In some embodiments, the method comprises administering to the subject an effective amount of one or more agents which selectively expands δ3 T cells. In some embodiments, the agent that selectively expands δ3 T-cells is selected from an agent which binds to the same, or essentially the same, epitope as, or competes with, an antibody selected from the group consisting of δ3-08, δ3-20, δ3-23, δ3-31, δ3-42, δ3-47 and δ3-58. In some embodiments, the agent that selectively expands δ3 T-cells is selected from an agent which binds to the same, or essentially the same, epitope as, or competes with, an antibody selected from the group consisting of δ3-08, δ3-23, δ3-31, δ3-42, δ3-47 and δ3-58. In some embodiments, the agent that selectively expands δ3 T-cells is selected from an agent which binds to the same, or essentially the same, epitope as, or competes with, an antibody selected from the group consisting of δ3-23, δ3-31, δ3-42, δ3-47 and δ3-58.

In some embodiments, the agent that selectively expands δ3 T-cells is an antibody or fragment thereof selected from the group consisting of δ3-08, δ3-20, δ3-23, δ3-31, δ3-42, δ3-47 and δ3-58. In some embodiments, the agent that selectively expands δ3 T-cells is an antibody or fragment thereof selected from the group consisting of δ3-08, δ3-23, δ3-31, δ3-42, δ3-47 and δ3-58. In some embodiments, the agent that selectively expands δ3 T-cells is an antibody or fragment thereof selected from the group consisting of δ3-23, δ3-31, δ3-42, δ3-47 and δ3-58.

In general the agent is a human or humanized agent, such as a human antibody or fragment thereof, or a humanized antibody or fragment thereof. In some cases, the agent that selectively expands M T cells, δ2 T cells, or δ3 T cells, or a combination thereof, is multivalent, preferably wherein the multivalent agent comprises at least two, or greater than two, antigen-binding-sites that specifically bind the same antigen, or wherein the multivalent agent comprises at least two, or greater than two, antigen-binding sites that specifically bind the same epitope of the same antigen. In some cases the agent that selectively expands δ1 T cells, δ2 T cells, or δ3 T cells, or a combination thereof is, or is at least, trivalent, tetravalent, or pentavalent. In some cases the agent that selectively expands δ1 T cells, δ2 T cells, or δ3 T cells, or a combination thereof is, or is at least, trivalent, tetravalent, or pentavalent, and is monospecific.

In some embodiments, the method comprises administering to the patient a population of engineered and/or non-engineered γδ T cells. In some embodiments, the method comprises administering the population of engineered and/or non-engineered γδ T cells before administering the one or more agents which selectively expand δ1 T cells, δ2 T cells, or δ3 T cells. In some embodiments, the method comprises administering the population of engineered and/or non-engineered γδ T cells after administering the one or more agents which selectively expand δ1 T cells, δ2 T cells, or δ3 T cells. In some embodiments, the method comprises administering to a subject the one or more agents which selectively expand δ1 T cells, δ2 T cells, or δ3 T cells, isolating a sample comprising in vivo expanded γδ T cells from the subject, optionally engineering one or more isolated γδ T cells, optionally expanding one or more isolated (e.g., and engineered) γδ T cells ex vivo, and then administering the population of isolated, engineered and/or non-engineered, and/or ex vivo expanded γδ T cells to the same or a different subject. In some embodiments, the administered population of engineered and/or non-engineered γδ T cells is a population of cells that are autologous to the subject.

In some embodiments, the administered population of engineered and/or non-engineered γδ T cells is a population of cells that are allogeneic to the subject. In some embodiments, the administered population of engineered and/or non-engineered γδ T cells is a population comprising at least 60% (e.g., at least 70%, 80%, or 90%; from about 60% to about 80%; or from about 60% to about 90%) δ1 γδ T cells, and the method comprises administering the γδ T cells and sequentially or simultaneously administering the one or more agents which selectively expand M T cells.

In some embodiments, the administered population of engineered and/or non-engineered γδ T cells is a population comprising at least 60% (e.g., at least 70%, 80%, or 90%; from about 60% to about 80%; or from about 60% to about 90%) 62 γδ T cells, and the method comprises administering the γδ T cells and sequentially or simultaneously administering the one or more agents which selectively expand δ2 T cells.

In some embodiments, the administered population of engineered and/or non-engineered γδ T cells is a population comprising at least 10% (e.g., at least 10%, 20%, 25%, 30%, 40%, 50%, 60%, 70%, 80%, or 90%; from about 10% to about 80%; from about 20% to about 40%; from about 20% to about 50%; or from about 20% to about 60%) δ3 γδ T cells, and the method comprises administering the γδ T cells and sequentially or simultaneously administering the one or more agents which selectively expand δ3 T cells.

In some embodiments, the method comprises administering an aminophosphonate (e.g., aminobisphosphonate) or a prenyl-phosphate. In some embodiments, the method comprises administering an aminophosphonate selected from the group consisting of zoledronate, pamidronic acid, alendronic acid, risedronic acid, ibandronic acid, and incadronic acid, or a salt thereof, and/or a hydrate thereof. In some embodiments, the method comprises repeatedly administering the one or more agents which selectively expand M T cells, δ2 T cells, and/or δ3 T cells. In some embodiments, the method comprises expanding or maintaining a population of administered γδ T cells in the subject. In some embodiments, the method comprises expanding, or maintaining a population of endogenous γδ T cells in the subject. In some embodiments, the method comprises activating, expanding or maintaining a population of endogenous γδ T cells in the subject, isolating a sample containing endogenous γδ T cells from the subject, manipulating the γδ T cells of the isolated sample (e.g., by purification, ex vivo expansion, and/or engineering), administering the manipulated γδ T cells to the same or different subject, and then activating, expanding, or maintaining a population of administered γδ T cells in the same or different subject by administering one or more selective in vivo activating agents.

In some embodiments, the method comprises expanding a population of endogenous and/or administered γδ T cells in the subject by a detectable amount (e.g., at least 10%, 20%, 30%, 40%, 50%, 60%, 75%, 2-fold, 10-fold, or 50-fold; or from about 10% to about 20%; from about 10% to about 50%; from about 10% to about 75%; from about 10% to about 2-fold; from about 10% to about 10-fold; from about 2-fold to about 10-fold; or from about 10-fold to about 50-fold) over an amount of γδ T cells in a subject that has not been administered the one or more agents which selectively expand δ1 T cells, δ2 T cells, and/or δ3 T cells. In some embodiments, the method comprises maintaining a larger population of endogenous and/or administered γδ T cells in the subject by a detectable amount (e.g., at least 10%, 20%, 30%, 40%, 50%, 60%, 75%, 2-fold, 10-fold, or 50-fold; or from about 10% to about 20%; from about 10% to about 50%; from about 10% to about 75%; from about 10% to about 2-fold; from about 10% to about 10-fold; from about 2-fold to about 10-fold; or from about 10-fold to about 50-fold) over the number of γδ T cells in a subject that has not been administered the one or more agents which selectively expand δ1 T cells, δ2 T cells, and/or δ3 T cells.

In some embodiments, the agent that selectively expands M T cells, δ2 T cells, or δ3 T cells, or a combination thereof is multivalent, preferably wherein the multivalent agent comprises at least two antigen-binding-sites that specifically bind the same antigen, or wherein the multivalent agent comprises at least two antigen-binding sites that specifically bind the same epitope of the same antigen. In some embodiments, the agent that selectively expands M T cells, δ2 T cells, or δ3 T cells, or a combination thereof is multivalent, preferably wherein the multivalent agent comprises at least three antigen-binding-sites that specifically bind the same antigen, or wherein the multivalent agent comprises at least three antigen-binding sites that specifically bind the same epitope of the same antigen. In some cases, the agent that selectively expands δ1 T cells, δ2 T cells, or δ3 T cells, or a combination thereof is, or is at least, bivalent, trivalent, tetravalent, or pentavalent. In some cases, the agent that selectively expands δ1 T cells, δ2 T cells, or δ3 T cells, or a combination thereof is, or is at least, trivalent, tetravalent, or pentavalent. In some cases, the agent that selectively expands M T cells, δ2 T cells, or δ3 T cells, or a combination thereof is, or is at least, tetravalent.

In some embodiments, the method comprises simultaneously or sequentially administering a cytokine to the subject. In some embodiments, the method comprises administering engineered γδ T cells to the subject, wherein the engineered γδ T cells comprise a transgene that encodes a secreted cytokine. In some embodiments, the cytokine is a common gamma chain cytokine, or a cytokine selected from the group consisting of IL-2, IL-15 and IL-4, preferably wherein the cytokine is IL-2, IL-15, and/or IL-4. In some embodiments, the method comprises administering a lymphodepletion protocol to the subject before administering γδ T cells.

In a second aspect, the present invention provides a method of treating a cancer, infectious disease, inflammatory disease, or an autoimmune disease in a subject in need thereof, the method comprising performing any one of the foregoing in vivo expansion methods and/or using any one or more of the γδ T cell activating agents described herein for in vivo expansion of γδ T cells in a subject in need thereof.

In a third aspect, the present invention provides a use of an agent that selectively expands δ1 T cells, δ2 T cells, or δ3 T cells in the manufacture of a medicament for the in vivo expansion of γδ T cells in a subject in need thereof. In some embodiments, the in vivo expansion of γδ T cells in a subject comprises treating a cancer, infectious disease, inflammatory disease, or an autoimmune disease in the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “Fig.” herein), of which:

FIG. 1 depicts heavy-chain framework and complementarity determining region amino acid sequences of δ1-specific MAbs.

FIG. 2 depicts light-chain framework and complementarity determining region amino acid sequences of the M-specific MAbs described in FIG. 1.

FIG. 3 depicts heavy-chain framework and complementarity determining region amino acid sequences of δ2-specific MAbs.

FIG. 4 depicts light-chain framework and complementarity determining region amino acid sequences of the δ2-specific MAbs described in FIG. 3.

FIG. 5 shows variable region sequences of δ3-specific anti-γδ TCR antibodies. Top sequence of heavy chain variable regions. Bottom sequence of light chain variable regions.

FIG. 6 shows an in vivo effect of γδ T cell sub-type specific activating agents on the number of total human CD45+ cells detected in mouse blood, bone marrow, lung and in the spleen after adoptive transfer human γδ T cells to the mice. Untreated mice were not treated with activating agents. Treated mice were treated with 3 or 10 μg activating agent as indicated. D1-35 refers to the δ1 γδ T cell-specific antibody δ1-35 described herein. Some animals also received intraperitoneal injection of non-specific murine IgG fraction 4-5 hrs prior to administration of cells, to saturate unoccupied Fc receptors.

FIG. 7A-B shows proliferation of adoptively transferred human γδ T cells in blood, bone marrow and spleen by 5 days after treatment with activating agent as evidenced by shift in the CellTrace Violet traces to the left toward decreased MFI due to dye dilution in cellular progeny at both 3 and 10 μg dose levels.

FIG. 8 shows an in vivo activating effect of indicated activating antibodies D1-35 and D1-08 (δ1-08), as detected by CellTrace Violet. Row 1, mice were not treated with activating agent. Row 2, mice were treated with activating agent D1-35. Row 3, mice were treated with activating agent and were not treated with non-specific murine IgG. Row 4, mice were treated with an hIgG4 isotype version of D1-35 activating agent. Row 5, mice were treated with activating agent D1-08.

FIGS. 9A-B shows in vivo proliferation of Vδ2 and Vδ3 cells detected in bone marrow and spleen of animals treated with D2-37 (δ2-37) and D3-23 (δ3-23) activating antibodies respectively, as detected by CellTrace Violet dye dilution.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as commonly understood to one of ordinary skill in the art to which the inventions described herein belong. For purposes of interpreting this specification, the following definitions will apply and whenever appropriate, terms used in the singular will also include the plural and vice versa. In the event that any definition set forth conflicts with any document incorporated herein by reference, the definition set forth below shall control.

The term “γδ T-cells (gamma delta T-cells)” as used herein refers to a subset of T-cells that express a distinct T-cell receptor (TCR), γδ TCR, on their surface, composed of one γ-chain and one δ-chain. The term “γδ T-cells” specifically includes all subsets of γδ T-cells and combinations thereof, including, without limitation, Vδ1, Vδ2, and Vδ3 γδ T cells, as well as naïve, effector memory, central memory, and terminally differentiated γδ T-cells. As a further example, the term “γδ T-cells” includes Vδ4, Vδ5, Vδ7, and Vδ8 γδ T cells, as well as Vγ2, Vγ3, Vγ5, Vγ8, Vγ9, Vγ10, and Vγ11 γδ T cells.

As used herein, the term “T lymphocyte” or “T cell” refers to an immune cell expressing CD3 (CD3+) and a T Cell Receptor (TCR+). T cells play a central role in cell-mediated immunity.

As used herein, the term “TCR” or “T cell receptor” refers to a dimeric heterologous cell surface signaling protein forming an alpha-beta or gamma-delta receptor. αβTCR recognize an antigen presented by an MHC molecule, whereas γδ TCR recognize an antigen independently of MHC presentation.

The term “MHC” (major histocompatibility complex) refers to a subset of genes that encodes cell-surface antigen-presenting proteins. In humans, these genes are referred to as human leukocyte antigen (HLA) genes. Herein, the abbreviations MHC or HLA are used interchangeably.

As used herein, the term “peripheral blood lymphocyte(s)” or “PBL(s)” is used in the broadest sense and refers to white blood cell(s) comprising T cells and B cells of a range of differentiation and functional stages, plasma cells, monocytes, macrophages, natural killer cells, basocytes, eosinophils, etc. The range of T lymphocytes in peripheral blood is about 20-80%.

As used herein, the term “cell population” refers to a number of cells obtained by isolation directly from a suitable source, usually from a mammal. The isolated cell population may be subsequently cultured in vitro. Those of ordinary skill in the art will appreciate that various methods for isolating and culturing cell populations for use with the present invention and various numbers of cells in a cell population that are suitable for use in the present invention. A cell population may be purified to homogeneity, substantial homogeneity, or to deplete one or more cell types (e.g., αβ T cells) by various culture techniques and/or negative or positive selection for a specified cell type. A cell population may be, for example, a mixed heterogeneous cell population derived from a peripheral blood sample, a cord blood sample, a tumor, a stem cell precursor, a tumor biopsy, a tissue, a lymph, skin, a sample of or containing tumor infiltrating lymphocytes, or from epithelial sites of a subject directly contacting the external milieu, or derived from stem precursor cells. Alternatively, the mixed cell population may be derived from in vitro cultures of mammalian cells, established from a peripheral blood sample, a cord blood sample, a tumor, a stem cell precursor, a tumor biopsy, a tissue, a lymph, skin, a sample of or containing tumor infiltrating lymphocytes, or from epithelial sites of a subject directly contacting the external milieu, or derived from stem precursor cells.

An “enriched” cell population or preparation refers to a cell population derived from a starting mixed cell population that contains a greater percentage of a specific cell type than the percentage of that cell type in the starting population. For example, a starting mixed cell population can be enriched for a specific γδ T-cell population. In one embodiment, the enriched γδ T-cell population contains a greater percentage of δ1 cells than the percentage of that cell type in the starting population. As another example, an enriched γδ T-cell population can contain a greater percentage of δ1 cells and a greater percentage of δ3 cells than the percentage of the respective cell type in the starting population. As yet another example, an enriched γδ T-cell population can contain a greater percentage of δ1 cells and a greater percentage of δ4 cells than the percentage of the respective cell type in the starting population. As another example, an enriched γδ T-cell population can contain a greater percentage of δ1 cells and a greater percentage of δ5 cells than the percentage of the respective cell type in the starting population. As yet another example, an enriched γδ T-cell population can contain a greater percentage of δ1 T cells, δ3 T cells, δ4 T cells, and δ5 T cells than the percentage of each of the respective cell type in the starting population. In another embodiment, the enriched γδ T-cell population contains a greater percentage of δ2 cells than the percentage of that cell type in the starting population. In another embodiment, the enriched γδ T-cell population contains a greater percentage of δ3 cells than the percentage of that cell type in the starting population. In yet another embodiment, the enriched γδ T-cell population contains a greater percentage of both M cells and δ2 cells than the percentage of the respective cell type in the starting population. In yet another embodiment, the enriched γδ T-cell population contains a greater percentage of δ1 cells, δ2 cells, and δ3 cells than the percentage of the respective cell type in the starting population. In all embodiments, the enriched γδ T-cell population contains a lesser percentage of αβ T-cell populations.

By “expanded” as used herein is meant that the number of the desired or target cell type (e.g., M and/or δ2 T-cells and/or δ3 T cells) in the enriched preparation is higher than the number in the initial or starting cell population.

By “selectively expand” is meant that the target cell type (e.g., δ1, δ2, or δ3 T-cells) are preferentially expanded over other non-target cell types, e.g., αβ T-cells or NK cells, or an untargeted subpopulation of γδ T cells. In certain embodiments, the activating agents of the invention selectively expand, e.g., engineered or non-engineered, δ1, δ2, and/or δ3 T-cells without, or without significant, expansion of αβ T-cells. In certain embodiments, the activating agents of the invention selectively expand, e.g., engineered or non-engineered, M T-cells without, or without significant, expansion of δ2 T-cells. In other embodiments, the activating agents of the invention selectively expand, e.g., engineered or non-engineered, δ2 T-cells without, or without significant, expansion of M T-cells. In certain embodiments, the activating agents of the invention selectively expand, e.g., engineered or non-engineered, δ3 T-cells without, or without significant, expansion of δ2 T-cells and/or without, or without significant, expansion of δ1 T-cells. In certain embodiments, the activating agents of the invention selectively expand, e.g., engineered or non-engineered, δ1 and δ3 T-cells without, or without significant, expansion of δ2 T-cells. In certain embodiments, the activating agents of the invention selectively expand, e.g., engineered or non-engineered, δ1 and δ4 T-cells without, or without significant, expansion of δ2 T-cells. In certain embodiments, the activating agents of the invention selectively expand, e.g., engineered or non-engineered, δ1 and δ5 T-cells without, or without significant, expansion of δ2 T-cells. In certain embodiments, the activating agents of the invention selectively expand, e.g., engineered or non-engineered, δ1, δ3, δ4 and δ5 T-cells without, or without significant, expansion of δ2 T-cells. In this context, the term “without significant expansion of” means that the preferentially expanded cell population are expanded at least 10-fold, preferably 100-fold, and more preferably 1,000-fold more than the reference cell population.

The term “admixture” as used herein refers to a combination of two or more isolated, enriched cell populations derived from a mixed, heterogeneous cell population. According to certain embodiments, the cell populations of the present invention are isolated γδ T cell populations. According to certain embodiments, the cell populations of the present invention are expanded ex vivo and/or provided in vitro and administered to a subject and thereby become in vivo γδ T cell populations. According to certain embodiments, the cell populations of the present invention are expanded in vivo by administering one or more agents that selectively expand a γδ T cell population.

The term “isolated,” as applied to a cell population, refers to a cell population, isolated from the human or animal body, which is substantially free of one or more cell populations that are associated with said cell population in vivo or in vitro.

The term “contacting” in the context of a cell population, as used here refers to incubation of an isolated cell population with a reagent, such as, for example, an antibody, cytokine, ligand, mitogen, or co-stimulatory molecule that can be linked either to beads or to cells. The antibody or cytokine can be in a soluble form, or it can be immobilized. In one embodiment, the immobilized antibody or cytokine is tightly bound or covalently linked to a bead or plate. In one embodiment, the antibody is immobilized on Fc-coated wells. In desirable embodiments, the contact occurs in vivo.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “directed against” is meant that the antibody reacts with one or more antigenic determinants of the desired antigen and does not react with other polypeptides or binds at much lower affinity (K_{D}>10⁻⁶ molar). Antibodies include, but are not limited to, polyclonal, monoclonal, chimeric, sdAb (heavy or light single domain antibody), single chain, F_{ab}, F_{ab′} and F_{(ab′)2} fragments, scFvs, diabodies, minibodes, nanobodies, and F_{ab} expression library.

An “effective amount” in the context of an in vivo method of expanding or maintaining an in vivo population of γδ T cells in a subject refers to a dose that produces an ascertainable increase in expansion or maintenance of the in vivo population of γδ T cells in a subject. As an example, the effective dose may selectively expand a target population of administered γδ T cells by a detectable amount (e.g., at least 1%, at least 5%, at least 10%, at least 25%, at least 50%, at least 75%, at least 2-fold, or from about 1% to about 10%, or from about 10% to about 2-fold). As another example, the effective dose may selectively expand a target population of endogenous in vivo γδ T cells by a detectable amount (e.g., at least 1%, at least 5%, at least 10%, at least 25%, at least 50%, at least 75%, at least 2-fold, or from about 1% to about 10%, or from about 10% to about 2-fold). As another example, the effective dose may maintain a larger number of viable target γδ T cells in the subject or in a tissue of the subject (e.g., a tumor tissue) as compared to a control subject that is not administered the one or more agents that selectively expand γδ T cells.

The term “effective amount,” as used herein in the context of an in vivo method of treating a cancer, infectious disease, inflammatory disease, or an autoimmune disease in a subject in need thereof, refers to the amount of a composition containing one or more agents that selectively expand γδ T cells administered to a subject, e.g., a human patient, already suffering from a disease, condition or disorder, sufficient to cure or at least partially arrest, or relieve to some extent one or more of the symptoms of the disease, disorder or condition being treated. The effectiveness of such compositions depend conditions including, but not limited to, the severity and course of the disease, disorder or condition, previous therapy, the patient's health status and response to the drugs, and the judgment of the treating physician. By way of example only, therapeutically effective amounts may be determined by routine experimentation, including but not limited to a dose escalation clinical trial.

The term “chimeric antigen receptors (CARs),” as used herein, may refer to artificial T-cell receptors, T-bodies, single-chain immunoreceptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain (allowing the T cell to activate upon engagement of targeting moiety with target cell, such as a target tumor cell), a transmembrane domain, and an extracellular domain that may vary in length and comprises a disease- or disorder-associated, e.g., a tumor-antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3-zeta, FcR, CD27, CD28, CD137, DAP 10/12, and/or OX40, ICOS, TLRs, etc. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “Fab” refers to an antibody fragment that consists of an entire L chain (V_{L} and C_{L}) along with the variable region domain of the H chain (V_{H}), and the first constant domain of one heavy chain (CH1). Papain digestion of an intact antibody can be used to produce two Fab fragments, each of which contains a single antigen-binding site. Typically, the L chain and H chain fragment of the Fab produced by papain digestion are linked by an interchain disulfide bond.

The term “Fc” refers to an antibody fragment that comprises the carboxy-terminal portions of both H chains (CH2 and CH3) and a portion of the hinge region held together by disulfide bonds. The effector functions of antibodies are determined by sequences in the Fc region; this region is also the part recognized by Fc receptors (FcR) found on certain types of cells. One Fc fragment can be obtained by papain digestion of an intact antibody.

The term “F(ab)₂” refers to an antibody fragment produced by pepsin digestion of an intact antibody. F(ab′)₂ fragments contain two Fab fragments and a portion of the hinge region held together by disulfide bonds. F(ab′)₂ fragments have divalent antigen-binding activity and are capable of cross-linking antigen.

The term Fab′ refers to an antibody fragment that is the product of reduction of an F(ab′)₂ fragment. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group.

The term “Fv” refers to an antibody fragment that consists of a dimer of one heavy-chain variable region and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The term “single-chain Fv” also abbreviated as “sFv” or “scFv” refer to antibody fragments that comprise the VH and VL antibody domains connected into a single polypeptide chain. Typically, the scFv polypeptide further comprises a polypeptide linker between the VH and VL domains, which enables the scFv to form the desired structure for antigen binding. For a review of scFv, see, e.g., Pluckthun, The Pharmacology of Monoclonal Antibodies, vol. 113, Rosenburg and Moore eds., Springer-Verlag, New York, pp. 269-315 (1994); and Malmborg et al., J. Immunol. Methods 183:7-13, 1995.

The expression “linear antibody” is used to refer to a polypeptide comprising a pair of tandem V_{H}-C_{H}1 segments (V_{H}-C_{H}1-V_{H}-C_{H}1) which form a pair of antigen binding regions. Linear antibodies can be bispecific or monospecific and are described, for example, by Zapata et al., Protein Eng. 8(10):1057-1062 (1995).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The term “hypervariable region,” “HVR,” or “HV,” refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

“Framework regions” (FR) are those variable domain residues other than the CDR residues. Each variable domain typically has four FRs identified as FR1, FR2, FR3, and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat. In certain instances, for the VL, the subgroup is subgroup kappa I as in Kabat. In certain instances, for the VH, the subgroup is subgroup III as in Kabat.

An antibody described herein can be humanized. “Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma and Immunol., 1:105-115 (1998); Harris, Biochem. Soc. Transactions, 23:1035-1038 (1995); Hurle and Gross, Curr. Op. Biotech., 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

An antigen-binding moiety described herein useful in activating an e.g., γδ, T cell, such as an antibody or antigen-binding fragment thereof as described herein, can be multivalent. For example, F(ab′)2 fragments have divalent antigen-binding activity and are capable of cross-linking antigen. Similarly, an antigen-binding moiety, such as an IgG or other canonical antibody architecture, can have a bivalent structure. In some cases, the antigen-binding moiety is greater than bivalent. In some cases, the antigen-binding moiety can be a trivalent moiety such as a trivalent antibody In some cases, the antigen binding moiety can be tetravalent such as a tetravalent antibody, e.g., an IgA antibody. In some cases, the antigen-binding moiety can have a valency of 10. For example, the antigen-binding moiety can be an IgM antibody. Preferred multivalent antigen-binding moieties described herein, e.g., antibodies or fragments thereof, typically bind the same antigen, and in some cases the same epitope of the same antigen, at each antigen-binding-site. In some cases, the multivalent antigen-binding moiety comprises at least one antigen-binding-site that is different from one other antigen-binding-site of the multivalent antigen-binding moiety.

As used herein, the “Kd” or “Kd value” refers to a dissociation constant measured by using surface plasmon resonance assays, for example, using a BIAcore™-2000 or a BIAcore™-3000 (BIAcore, Inc., Piscataway, N.J.) at 25° C. with CM5 chips immobilized with antigen or antibody at about 10 response units (RU). For divalent or other multivalent antibodies, typically the antibody is immobilized to avoid avidity-induced interference with measurement of the dissociation constant. For further details see, e.g., Chen et al., J. Mol. Biol. 293:865-881 (1999).

“Or better” when used herein to refer to binding affinity refers to a stronger binding between a molecule and its binding partner. “Or better” when used herein refers to a stronger binding, represented by a smaller numerical KD value. For example, an antibody which has an affinity for an antigen of “0.6 nM or better”, the antibody's affinity for the antigen is <0.6 nM, i.e. 0.59 nM, 0.58 nM, 0.57 nM etc. or any value less than or equal to 0.6 nM.

The term “epitope” includes any protein determinant, lipid or carbohydrate determinant capable of specific binding to an immunoglobulin or T-cell receptor. Epitopic determinants usually consist of active surface groupings of molecules such as amino acids, lipids or sugar side chains and usually have specific three dimensional structural characteristics, as well as specific charge characteristics. An antibody is said to specifically bind an antigen when the equilibrium dissociation constant (KD) is within the range of 10⁻⁶-10⁻¹² M, or better. Specific binding can refer to binding to a target epitope with at least a 10-fold; preferably 100-fold; or more preferably 1,000-fold tighter dissociation constant (lower KD), as compared to the dissociation constant for binding to other non-target epitopes. In some cases, the target epitope is an epitope of a δ1, δ2, or δ3 chain of a delta-3 TCR. In some cases, the non-target epitope is an αβ TCR. In some cases, the non-target epitope is a different sub-type delta chain. Specificity of binding can be determined in the context of binding to a extracellular region of a γδ-TCR and/or αβ-TCR (e.g., as an Fc fusion immobilized on an ELISA plate or as expressed on a cell).

An “activating epitope” is capable of activation of the specific γδ T-cell population upon binding. T cell proliferation indicates T cell activation and expansion.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in a number of different formats, using either labeled antigen or labeled antibody. In some embodiments, the antigen is immobilized on a 96-well plate, and the ability of unlabeled antibodies to block the binding of labeled antibodies is measured using radioactive or enzyme labels. Alternatively, the competition studies, using labelled and unlabeled antibodies, are performed using flow cytometry on antigen-expressing cells.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Epitope binning”, as defined herein, is the process of grouping antibodies based on the epitopes they recognize. More particularly, epitope binning comprises methods and systems for discriminating the epitope recognition properties of different antibodies, combined with computational processes for clustering antibodies based on their epitope recognition properties and identifying antibodies having distinct binding specificities.

An “agent” or “compound” according to the present invention comprises small molecules, polypeptides, proteins, antibodies or antibody fragments. Small molecules, in the context of the present invention, mean in one embodiment chemicals with molecular weight smaller than 1000 Daltons, particularly smaller than 800 Daltons, more particularly smaller than 500 Daltons. The term “therapeutic agent” refers to an agent that has biological activity. The term “anti-cancer agent” refers to an agent that has biological activity against cancer cells.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including stem cells, blood cells, embryonic cord blood cells, tumor cells, transduced cells, etc.

The terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease (e.g., decrease of tumor size, tumor burden, or tumor distribution), stabilized (i.e., not worsening) state of disease, delay or slowing of disease progression, amelioration or palliation of the disease state, and remission (whether partial or total), whether detectable or undetectable. “Treatment” can also mean prolonging survival, as compared to expected survival if not receiving treatment. Those in need of treatment include those already with the condition or disorder as well as those prone to have the condition or disorder or those in which the condition or disorder is to be prevented.

Administration “in combination with” one or more further therapeutic agents includes simultaneous (concurrent) and consecutive administration in any order.

The term “identical,” as used herein, refers to two or more sequences or subsequences that are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75 to about 100 nucleic acids in length, over a region that is about 50 nucleic acids in length, or, where not specified, across the entire sequence of a polynucleotide sequence.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “subject,” or “patient”, as used herein, refers to a vertebrate. In certain embodiments, the vertebrate is a mammal. Mammals include, but are not limited to, humans, non-human primates, farm animals (such as cows), sport animals, and pets (such as cats, dogs, and horses). In certain embodiments, a mammal is a human.

The term antigen presenting cell (APC) refers to a wild-type APC, or an engineered or artificial antigen presenting cell (aAPC). APCs can be provided as an irradiated population of APCs. APCs can be provided from a immortalized cell line (e.g., K562 or an engineered aAPC derived from an immortalized cell line) or as a fraction of cells from a donor (e.g., PBMCs).

As used herein, the terms “structurally different” and “structurally distinct,” in reference to a protein or polypeptide fragment thereof, or an epitope, refer to a covalent (i.e., structural) difference between at least two different proteins, polypeptide fragments thereof, or epitopes. For example, two structurally different proteins (e.g., antibodies) can refer to two proteins that have different primary amino acid sequences. In some cases, structurally different activating agents bind structurally different epitopes, such as epitopes having a different primary amino acid sequence.

As used herein, the term “anti-tumor cytotoxicity” that is “independent of” a specified receptor activity (e.g., NKp30 activity, NKp44 activity, and/or NKp46 activity), refers to anti-tumor cytotoxicity that is exhibited whether or not the specified receptor or specified combination of receptors is expressed by the cell or functional. As such, a γδ T-cell that exhibits anti-tumor cytotoxicity that is independent of NKp30 activity, NKp44 activity, and/or NKp46 activity can also exhibit NKp30 activity-dependent anti-tumor cytotoxicity, NKp44 activity-dependent anti-tumor cytotoxicity, and/or NKp46 activity-dependent anti-tumor cytotoxicity.

As used herein, the terms “NKp30 activity-dependent anti-tumor cytotoxicity,” “NKp44 activity-dependent anti-tumor cytotoxicity,” and “NKp46 activity-dependent anti-tumor cytotoxicity” refer to anti-tumor cytotoxicity that requires functional expression of the specified receptor. The presence or absence of such receptor dependent anti-tumor cytotoxicity can be determined by performing standard in vitro cytotoxicity assays, such as performed in Example 48 of PCT/US17/32530, in the presence or absence of an antagonist to the specified receptor. For example, presence or absence of NKp30 activity-dependent anti-tumor cytotoxicity can be determined by comparing the results of an in vitro cytotoxicity assays in the presence of an anti-NKp30 antagonist to the results obtained in the absence of an anti-NKp30 antagonist.

Moreover, it is understood that a γδ T-cell or population of γδ T-cells can be assayed for mRNA expression of the one or more cytotoxicity receptors NKp30, NKp44, and/or NKp46. In such cases, an expression assay can indicate presence or absence of receptor dependent anti-tumor cytotoxicity. For example, the measured mRNA expression of the γδ T-cell or population of γδ T-cells can be compared to a positive control using a cell or cell-line that does exhibit the specified receptor dependent cytotoxicity (e.g., as verified by an in vitro cytotoxicity assay in the presence and absence of an antagonist).

As used herein, a γδ T-cell population that comprises anti-tumor cytotoxicity, wherein at least a specified “%” of the anti-tumor cytotoxicity is “independent of” a specified receptor activity (e.g., NKp30 activity, NKp44 activity, and/or NKp46 activity), refers to a cell where blocking specified receptor reduces measured anti-tumor cytotoxicity by no more than the numerical % value. Thus, a γδ T-cell population that comprises anti-tumor cytotoxicity, wherein at least 50% of the anti-tumor cytotoxicity is independent of NKp30 activity would exhibit a reduction of 50% or less of in vitro anti-tumor cytotoxicity in the presence of an NKp30 antagonist as compared to in the absence of the NKp30 antagonist.

Overview

In humans, γδ T-cell(s) are a subset of T-cells that provide a link between the innate and adaptive immune responses. These cells undergo V-(D)-J segment rearrangement to generate antigen-specific γδ T-cell receptors (γδ TCRs), and γδ T-cell(s) and can be directly activated via the recognition of an antigen by either the γδ TCR or other, non-TCR proteins, acting independently or together to activate γδ T-cell effector functions. γδ T-cells represent a small fraction of the overall T-cell population in mammals, approximately 1-5% of the T-cells in peripheral blood and lymphoid organs, and they appear to reside primarily in epithelial cell-rich compartments like skin, liver, digestive, respiratory, and reproductive tracks. Unlike αβ TCRs, which recognize antigens bound to major histocompatibility complex molecules (MHC), γδ TCRs can directly recognize bacterial antigens, viral antigens, stress antigens expressed by diseased cells, and tumor antigens in the form of intact proteins or non-peptide compounds.

TS-1, TS8.2, B6, and 15D can activate γδ T cells. Without being bound by theory, different levels of activation and expansion of cultures originating from different donors may be due to the donor γδ variable TCR repertoire and the specificity of the antibody binding epitope. It has been discovered that not every agent which binds to specific γδ T-cell subsets is capable of activating the specific γδ T-cell and particularly activating the specific γδ T-cell population to clinically-relevant levels, i.e., >10⁸ target γδ T cells in an enriched culture. Similarly, not every binding epitope of a γδ T-cell population is an activating epitope, i.e., capable of activation of the specific γδ T-cell population upon binding.

The inventors of the present invention have identified specific γδ variable TCR binding regions associated with potent activation of specific γδ T cell subtypes thus enabling the specific in vivo or ex vivo activation and expansion of γδ T cell subtypes. Ex vivo activation and expansion by binding to the identified TCR binding regions can be used to produce clinically relevant levels of highly enriched γδ T-cell populations with increased purity, and admixtures thereof, that can be administered to patients. In vivo activation can be used to expand and/or maintain an administered γδ T cell population (or one or more sub-types thereof), induce the expansion of an endogenous γδ T cell population (or one or more sub-types thereof), or a combination thereof. In some cases, the in vivo activation can be used to expand an endogenous γδ T cell population in vivo, and a portion of the expanded population can be isolated and expanded ex vivo using the methods described herein. The in vivo expanded, isolated, and ex vivo expanded, γδ T cells can be stored, optionally engineered, and/or administered to a subject in need thereof. The engineering can be performed before ex vivo expansion or after ex vivo expansion. The administered cells can be in vivo expanded and/or maintained by administering to the subject one or more agents that selectively expand γδ T cells.

Activating ligands, including antibodies or other binding agents, which specifically bind the activating epitopes capable of inducing enhanced activation and expansion of γδ T cell subtypes are also contemplated and further described herein. In some embodiments, the activating agents used in the methods and compositions described herein, including methods of in vivo expansion of γδ T cells and/or administering to a subject in need thereof, are the agents described in PCT/US2015/061189 and/or PCT/US17/32530 for ex vivo expansion. In some embodiments, the activating agents used in the methods and compositions described herein, including methods of in vivo expansion of γδ T cells and/or administering to a subject in need thereof, are the δ3 specific activating agents described in PCT/US18/061384 for ex vivo expansion. PCT/US17/32530 and PCT/US18/061384 are incorporated by reference in the entirety for all purposes including all disclosures related to γδ T cell activating agents, γδ T cell compositions, and methods of γδ T cell activation, γδ T cell expansion, treatment, administration, and dosing.

In some aspects, the instant invention provides ex vivo methods for expansion of engineered or non-engineered γδ T-cells. Generally, the ex vivo expansion methods are used in combination with the in vivo expansion and/or maintenance methods for expansion of engineered or non-engineered γδ T-cells described herein. For example, γδ T-cells can be selectively expanded in vivo by administration of one or more agents that selectively expand γδ T-cells or one or more sub-populations thereof. A portion of the γδ T-cells selectively expanded in vivo can be isolated and then further, e.g., selectively, expanded ex vivo. In some cases, ex vivo expanded γδ T-cells, whether or not previously expanded in vivo, can be administered to a subject in need thereof. In some cases, the ex vivo expanded γδ T-cells, or a portion thereof, are administered to the same subject from which the initial population was isolated. In some cases, the ex vivo expanded γδ T-cells, or a portion thereof, are administered to a different subject from which the initial population was isolated. In some cases, the administered ex vivo expanded γδ T-cells are further expanded or maintained in vivo by administering to the subject one or more agents that selectively expand γδ T-cells.

In-Vivo Expansion of γδ T-Cells

The present disclosure provides methods for the in vivo expansion and/or maintenance of a population of non-engineered or engineered γδ T-cells. A non-engineered or engineered γδ T-cell of the disclosure may be additionally activated and/or expanded ex vivo before and/or after in vivo expansion or maintenance. In some embodiments, the in vivo activation, expansion and/or maintenance of a non-engineered or engineered γδ T-cell of the disclosure can be performed without administering an aminophosphonate or a prenyl-phosphate. In some embodiments, the in vivo activation, expansion and/or maintenance of a non-engineered or engineered γδ T-cell of the disclosure can be performed, at least in part, by administering an aminophosphonate or a prenyl-phosphate. For example, the in vivo activation and/or expansion can be performed by a method comprising administering one or more agents that selectively expand a non-engineered or engineered γδ T-cell of the disclosure by binding to an epitope specific of a 61, 62, or 63 γδ T cell, or a combination thereof, wherein the method further comprises administering an aminophosphonate or a prenyl-phosphate.

Generally, the methods include administering to the subject one or more agents that selectively expand γδ T-cells to the subject in an effective amount. In some embodiments, the methods include providing a pharmaceutical composition comprising a γδ T-cell population and one or more agents that selectively expand γδ T cells, and administering the provided pharmaceutical composition to the subject. In some embodiments, the methods include administering γδ T cells to the subject and then administering one or more agents that selectively expand γδ T-cells. In some embodiments, the methods include administering one or more agents that selectively expand γδ T-cells and then administering γδ T cells to the subject. In certain embodiments, γδ T-cells are administered, one or more times, and one or more agents that selectively expand γδ T-cells are periodically administered to the subject in an effective amount to expand or maintain the administered γδ T-cells.

In some cases, the methods include administering the one or more agents that selectively expand γδ T-cells to the subject multiple times, e.g., at least twice, at least three times, or at least four times. In some cases, the methods include administering the one or more agents that selectively expand γδ T-cells to the subject from 1 to 2 times, from 1 to 3 times, from 1 to 5 times, from 1 to 10 times, from 2 to 4 times, from 2 to 10 times, or from 5 to 10 times. In some cases, the methods include administering the one or more agents that selectively expand γδ T-cells to the subject with dosage period of from two to four weeks, from two to six weeks, one month (e.g., about 30 days), two months, or from one to two months. In some cases, the methods include administering the one or more agents that selectively expand γδ T-cells to the subject periodically for at least two months (e.g., about 60 days), at least three months (e.g., about 90 days), at least four months (e.g., about 120 days), or at least six months (e.g., 180 days about).

A method of the invention can expand various γδ T-cell populations, such as a Vγ1⁺, a Vγ2⁺, or Vγ3⁺ γδ T-cell population in vivo. In some cases, a method of the invention can expand a Vδ1⁺ T-cell population; a Vδ1⁺ and a Vδ3⁺ T-cell population; a Vδ1⁺ and a Vδ4⁺ T-cell population; a Vδ1⁺ and a Vδ2⁺ T-cell population; or a Vδ1⁺, Vδ3⁺, Vδ4⁺, and a Vδ5⁺ T-cell population.

In some instances, a γδ T-cell population can be expanded in vivo by a detectable amount (e.g., at least 10%, at least 25%, at least 50%, at least 75%, at least 2-fold, or from about 10% to about 2-fold) in fewer than 45 days, fewer than 40 days, fewer than 35 days, or fewer than 30 days. In some instances, a γδ T-cell population can be expanded in vivo by from about 10% to about 10-fold, from about 50% to about 10-fold, or more in fewer than 45 days, fewer than 40 days, fewer than 35 days, or fewer than 30 days. In some instances, a γδ T-cell population can be expanded in vivo by about 10 to about 50-fold in fewer than 45 days, fewer than 40 days, fewer than 35 days, or fewer than 30 days.

In some aspects, provided are methods for selectively expanding various γδ T-cells in vivo, including engineered and non-engineered γδ T-cells by contacting the γδ T-cells with an activation agent. In some cases, the activation or activating agent binds to a specific epitope on a cell-surface receptor of a γδ T-cell. The activation agent can be an antibody, such as a monoclonal antibody. The activation agent can specifically activate the growth of one or more types of γδ T-cells, such δ1; δ2; δ3; M and δ3; δ1 and δ4; δ1 and δ5; δ1, δ3, and δ4; or δ1, δ3, δ4, and δ5 cell populations, or combinations thereof. In some embodiments the activation agent specifically activates the growth of δ1 cell populations to expand or maintain a M T-cell population in vivo. In other cases, the activation agent specifically activates the growth of δ2 cell populations to expand or maintain a δ2 T-cell population in vivo. In other cases, the activation agent specifically activates the growth of δ3 cell populations to expand or maintain a δ3 T-cell population in vivo. In other cases, the activation agent specifically activates the growth of δ1 and δ3 cell populations to expand or maintain a M and δ3 T-cell population in vivo. In other cases, the activation agent specifically activates the growth of M and δ4 cell populations to expand or maintain a M and δ3 T-cell population in vivo. In other cases, the activation agent specifically activates the growth of δ1 and δ5 cell populations to expand or maintain a δ1 and δ5 T-cell population in vivo.

In some cases, the agent that selectively expands M T cells, δ2 T cells, or δ3 T cells, or a combination thereof, is multivalent, preferably wherein the multivalent agent comprises at least two antigen-binding-sites that specifically bind the same antigen, or wherein the multivalent agent comprises at least two antigen-binding sites that specifically bind the same epitope of the same antigen. In some cases, the agent that selectively expands M T cells, δ2 T cells, or δ3 T cells, or a combination thereof, comprises at least three antigen-binding-sites that specifically bind the same antigen, or wherein the multivalent agent comprises at least three antigen-binding sites that specifically bind the same epitope of the same antigen. In some cases the agent that selectively expands M T cells, δ2 T cells, or δ3 T cells, or a combination thereof is, or is at least, bivalent, trivalent, tetravalent, or pentavalent.

One or more activation agent can contact the γδ T-cells in vivo (for example an activator γδ T cell innate receptor) and simultaneously or sequentially a costimulatory molecule can contact the γδ T-cells to provide further stimulation and to expand the γδ T-cells. In some embodiments, the activation agent and/or costimulatory agent can be lectins of plant and non-plant origin, monoclonal antibodies that activate γδ T-cells, and other non-lectin/non-antibody agents. In other cases, the plant lectin can be concanavalin A (ConA) although other plant lectins such as may be used. Other examples of lectins include protein peanut agglutinin (PNA), soybean agglutinin (SBA), Les culinaris agglutinin (LCA), Pisum sativum agglutinin (PSA), Helix pomatia agglutinin (HPA), Vicia graminea Lectin (VGA), Phaseolus Vulgaris Erythroagglutinin (PHA-E), Phaseolus Vulgaris Leucoagglutinin (PHA-L), Sambucus Nigra Lectin (SNA, EBL), Maackia Amurensis, Lectin II (MAL II), Sophora Japonica Agglutinin (SJA), Dolichos Biflorus Agglutinin (DBA), Lens Culinaris Agglutinin (LCA), Wisteria Floribunda Lectin (WFA, WFL).

Non-limiting examples of activating agents and costimulatory molecules include any one or more antibodies selective for a δ or γ-chain or subtypes thereof described herein, antibodies such as 5A6.E9, B1, TS8.2, 15D, B6, B3, TS-1, γ3.20, 7A5, IMMU510, R9.12, 11F2, or a combination thereof. Other examples of activating agents and costimulatory molecules include zoledronate, phorbol 12-myristate-13-acetate (TPA), mezerein, staphylococcal enterotoxin A (SEA), streptococcal protein A, or a combination thereof.

In other cases, the activation agent and/or costimulatory agent can be, antibodies or ligands to α TCR, β TCR, γ TCR, δ TCR, CD277, CD28, CD46, CD81, CTLA4, ICOS, PD-1, CD30, NKG2D, NKG2A, HVEM, 4-1 BB (CD137), OX40 (CD134), CD70, CD80, CD86, DAP, CD122, GITR, FcεRIγ, CD1, CD16, CD161, DNAX, accessory molecule-1 (DNAM-1), one or more NCRs (e.g., NKp30, NKp44, NKp46), SLAM, Coxsackie virus and adenovirus receptor or a combination thereof.

In vivo activation, expansion and/or maintenance of γδ T-cells can be performed using activation and co-stimulatory agents described herein to trigger specific γδ T-cell proliferation and persistent populations. In one aspect, agents that provide specific γδ T cell activating signals can be different monoclonal antibodies (MAbs) directed against the γδ TCRs.

In one aspect, the MAbs can bind to different epitopes on the constant or variable regions of δ TCR and/or γ TCR. In one aspect, the MAbs can include γδ TCR pan MAbs. In one aspect, the γδ TCR pan MAbs may recognize domains shared by different γ and δ TCRs on either the γ or δ chain or both, including δ1, δ2, and δ3 T cell populations. In one aspect, the antibodies may be 5A6.E9 (Thermo scientific), B1 (Biolegend), IMMU510 and/or 11F2 (11F2) (Beckman Coulter).

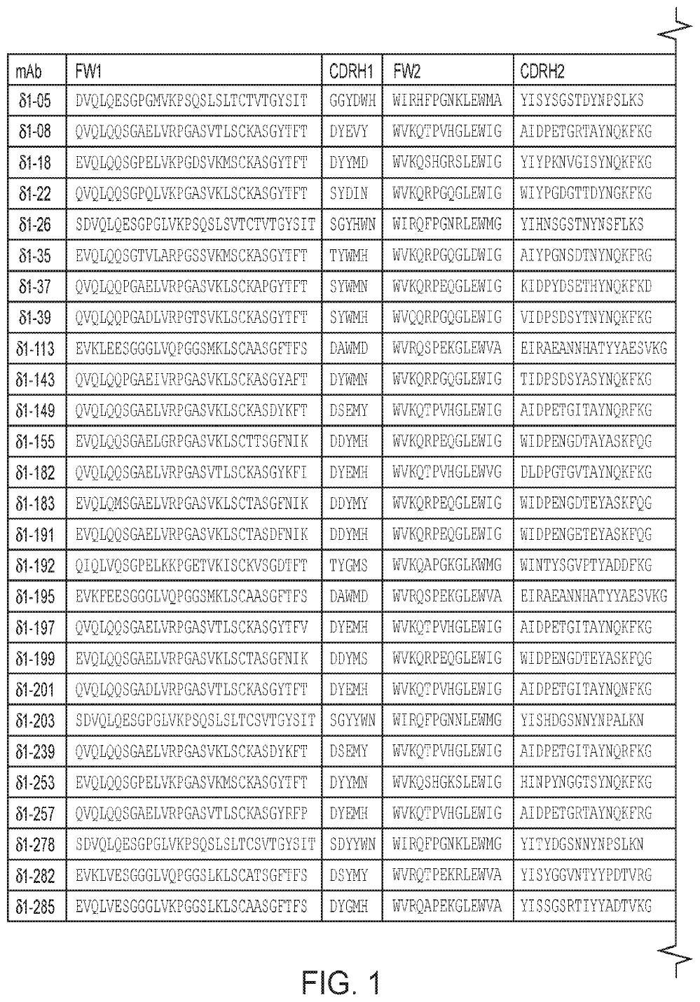

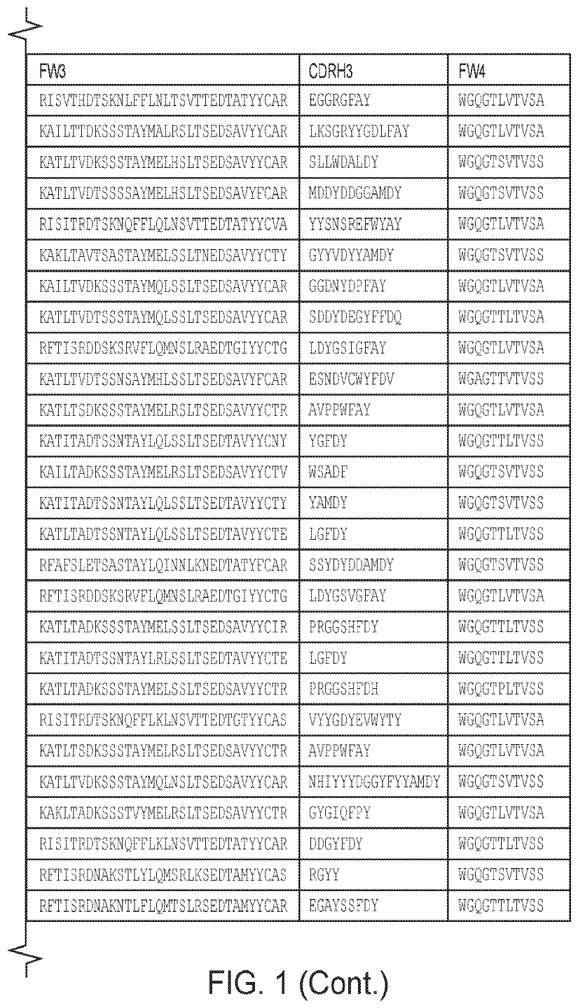

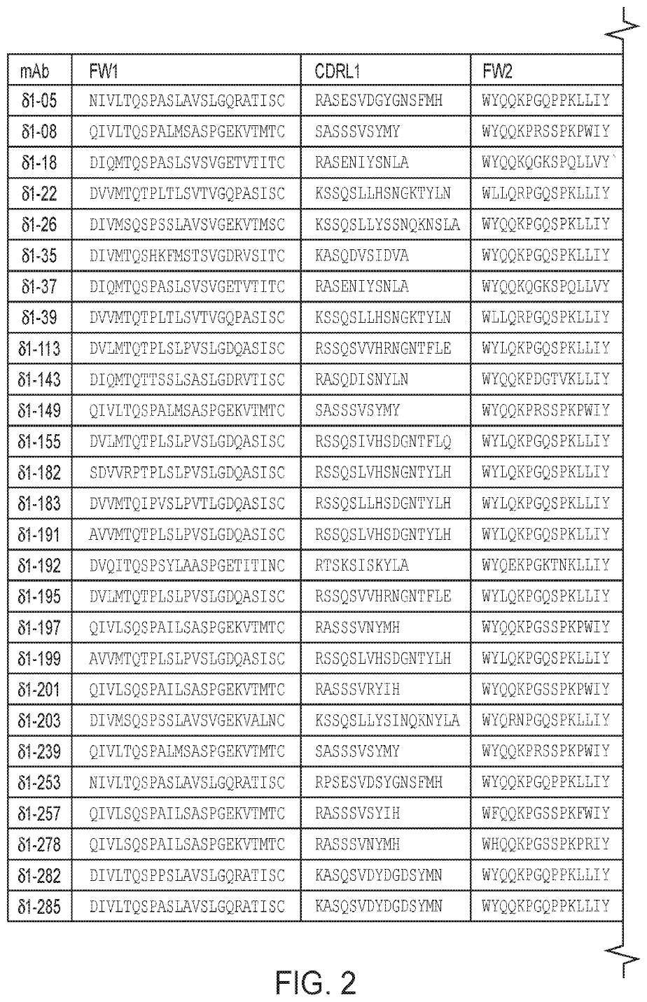

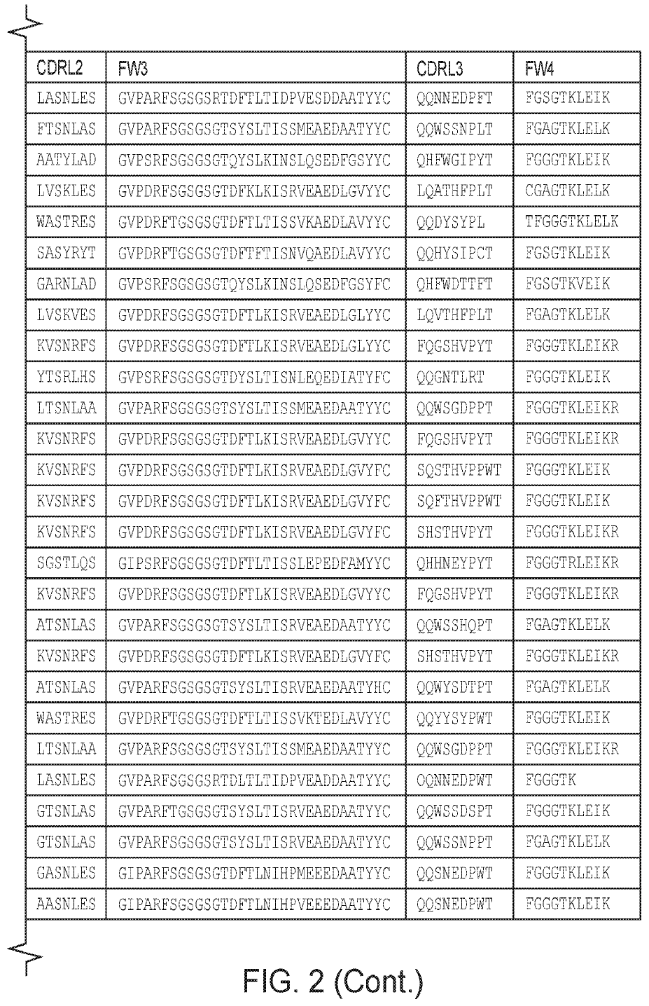

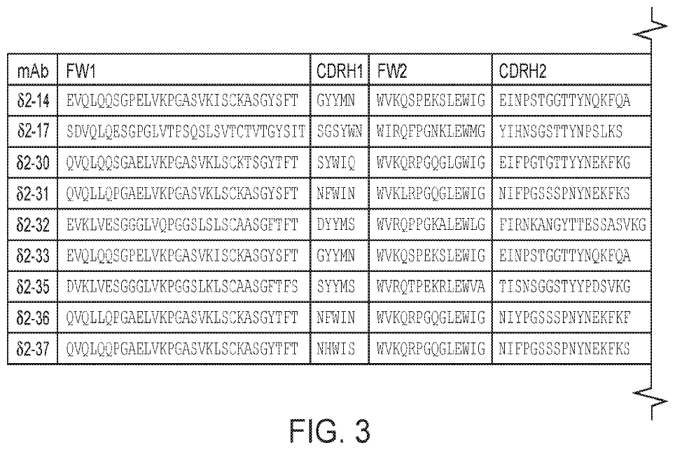

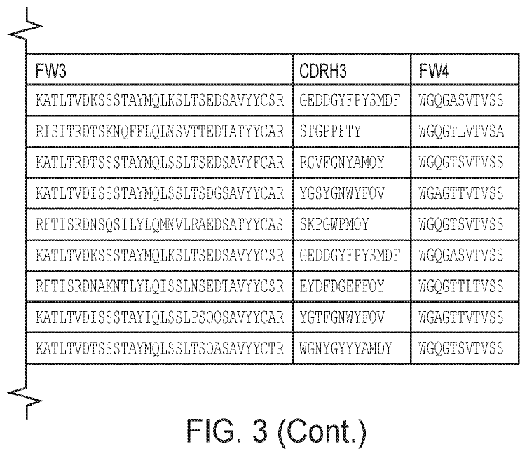

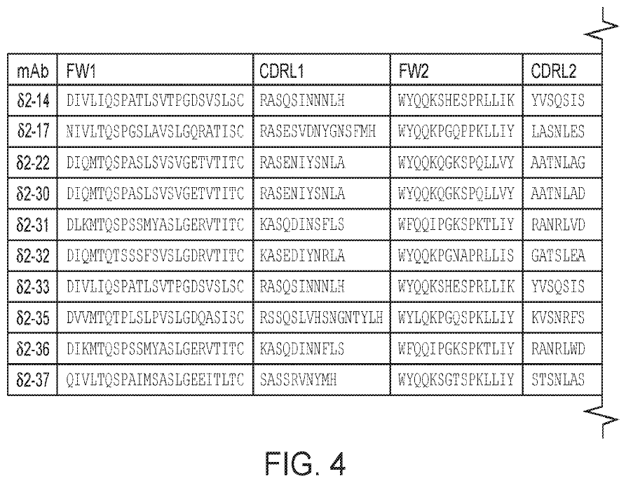

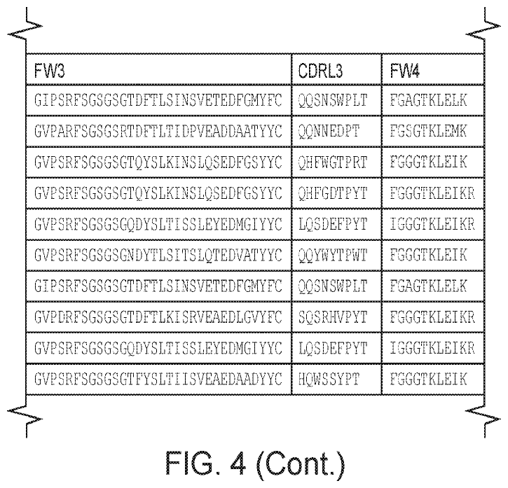

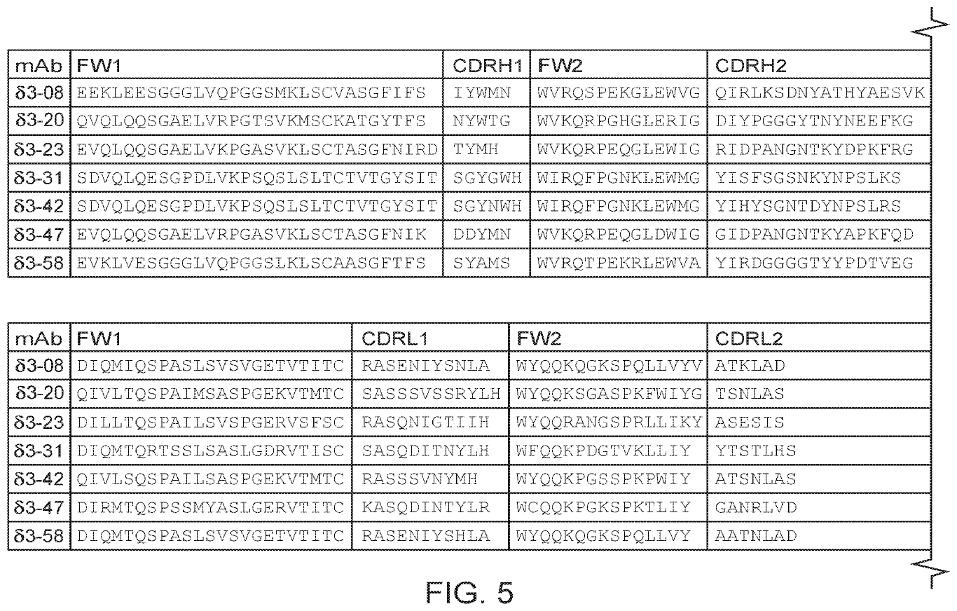

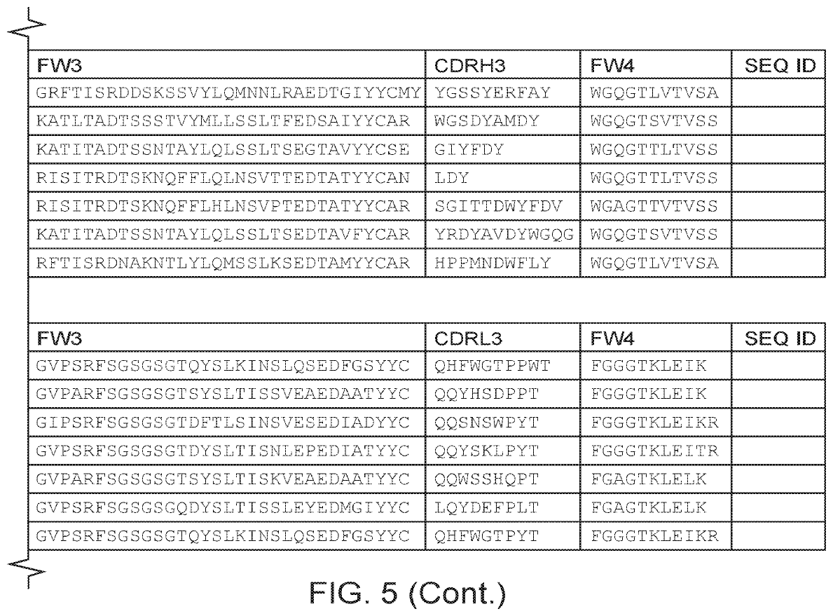

In one aspect, the MAbs can be directed to specific domains unique to the variable regions of the γ chain (7A5 Mab, directed to Vγ9 TCR (Thermo Scientific #TCR1720)), or domains on Vδ1 variable region (Mab TS8.2 (Thermo scientific #TCR1730; MAb TS-1 (ThermoFisher #TCR 1055), MAb R9.12 (Beckman Coulter #IM1761)), or Vδ2 chain (MAb 15D (Thermo Scientific #TCR1732 or Life technologies #TCR2732) B6 (Biolegend #331402), one of the M-#antibodies described in FIGS. 1-2, one of the δ2-#antibodies described in FIGS. 3-4, or one of the δ3-#antibodies described in FIG. 5.

In certain embodiments, the activation agents are agents that bind the same or essentially the same epitope as one of the δ1-#antibodies described in FIGS. 1-2, one of the δ2-#antibodies described in FIGS. 3-4, or one of the δ3-#antibodies described in FIG. 5. In certain embodiments, the activation agents are agents that compete with one of the δ1-#antibodies described in FIGS. 1-2, one of the δ2-#antibodies described in FIGS. 3-4, or one of the δ3-#antibodies described in FIG. 5. Additional activation agents are described herein, and in PCT/US17/32530, filed May 12, 2017, and Attorney Docket No. ADC-0003-PR1, U.S. Provisional Appl. No. 62/586,782, which was co-filed with the present application.

In certain embodiments, in vivo activation, expansion and/or maintenance can be further supported by simultaneously or sequentially administering a cytokine or other stimulating agent such as IL-2, IL-4, IL-7, IL-9, IL-12, IL-15, IL-18, IL-19, IL-21, IL 23, IL-33, IFNγ, granulocyte-macrophage colony stimulating factor (GM-CSF), or granulocyte colony stimulating factor (G-CSF). In some cases, the cytokine is IL-2, IL-15, IL-12, or IL-21. In some cases, the cytokine is IL-2. In some cases, the cytokine is IL-15. In some cases, the cytokine is IL-4. In some cases, the cytokine is a common gamma chain cytokine selected from the group consisting of IL-2, IL-4, IL-7, IL-9, IL-15, and IL-21, or a combination thereof.

In certain embodiments, in vivo expansion or maintenance can be further supported by administering a lymphodepletion protocol to the subject before administering activating agent and/or before administering the γδ T cells (e.g., γδ CAR-T cells). In some cases, the lymphodepletion protocol comprises administering fludarabine and cyclophosphamide. In some cases, the lymphodepletion comprises or further comprises leukapharesis. In some cases, the lymphodepletion comprises or further comprises bendamustine. In some cases, lymphodepletion is not administered to a patient that is, is diagnosed as, or is suspected of being, lymphocytopenic at the time CAR T cells are administered.

Isolation of γδ T-Cells

In some embodiments, the instant invention provides ex vivo methods for producing enriched γδ T-cell populations from isolated mixed cell populations, comprising contacting the mixed cell population with one or more agents which selectively expand δ1 T-cells; δ1 T-cells and δ3 T-cells; δ1 T-cells and δ4 T-cells; or δ1, δ3, δ4, and δ5 T cells by binding to an epitope specific of a 61 TCR; a δ1 and δ4 TCR; or a δ1, δ3, δ4, and δ5 TCR respectively to provide an enriched γδ T cell population. In other aspects, the instant invention provides ex vivo methods for producing enriched γδ2 T-cell populations from isolated mixed cell populations, comprising contacting the mixed cell population with one or more agents which selectively expand δ2 T-cells by binding to an epitope specific of a δ2 TCR to provide an enriched γδ2 T cell population. In other aspects, the instant invention provides ex vivo methods for producing enriched γδ3 T-cell populations from isolated mixed cell populations, comprising contacting the mixed cell population with one or more agents which selectively expand δ3 T-cells by binding to an epitope specific of a δ3 TCR to provide an enriched γδ3 T cell population.

In other aspects, the present disclosure provides methods for the genetic engineering of γδ T-cells that have been isolated from a subject. Methods of enrichment, expansion, purification by, e.g., positive and/or negative selection, or genetic engineering can be performed singly or in combination, in any order. In one embodiment, γδ T-cells can be expanded in vivo in a subject, isolated from the subject, genetically engineered, and then expanded ex vivo, and optionally administered to a subject. In another embodiment, γδ T-cells can be isolated from a subject, genetically engineered, optionally activated and expanded ex vivo, administered to a subject, and then expanded or maintained in vivo. In some cases, the subject from which γδ T-cells are isolated and the subject to which γδ T-cells are administered is the same subject. In some cases, the subject from which γδ T-cells are isolated and the subject to which γδ T-cells are administered is a different subject.

An engineered or non-engineered, γδ T-cell population can be expanded, e.g. directly, from a complex sample of a subject. In some case, the complex sample is isolated and expanded ex vivo by directly contacting the complex sample with one or more agents that selectively expand the target γδ T-cell population. In some cases, the complex sample is isolated and then purified by positive or negative selection before ex vivo expansion is performed.

A complex sample can be a peripheral blood sample (e.g., PBLs or PBMCs), a leukapheresis sample, a cord blood sample, a tumor, a stem cell precursor, a tumor biopsy, a tissue, a lymph, or from epithelial sites of a subject directly contacting the external milieu, or derived from stem precursor cells. In some cases, the present disclosure provides methods for selective expansion of Vδ1⁺ cells, Vδ2⁺ cells, Vδ3⁺ cells, Vδ1⁺ cells and Vδ3⁺ cells, Vδ1⁺ cells and Vδ4⁺ cells, Vδ1⁺ cells, Vδ3⁺ cells, Vδ4⁺ cells, and Vδ5⁺ cells, or any combination thereof.

Peripheral blood mononuclear cells can be collected from a subject, for example, with an apheresis machine, including the Ficoll-Paque™ PLUS (GE Healthcare) system, or another suitable device/system. γδ T-cell(s), or a desired subpopulation of γδ T-cell(s), can be purified from the collected sample with, for example, flow cytometry techniques. Cord blood cells can also be obtained from cord blood during the birth of a subject. See WO 2016/081518, incorporated by reference herein in its entirety for all purposes including but not limited to methods and compositions for PBMC isolation, γδ T cell activation, and making and using γδ T cell activation agents.

A γδ T-cell may be expanded from an isolated complex sample or mixed cell population that is cultured in vitro by contacting the mixed cell population with one or more agents which expand γδ T-cell by specifically binding to an epitope of a γδ TCR to provide an enriched γδ T-cell population, e.g., in a first enrichment step. In some embodiments, γδ T cells comprised in a whole PBMC population, without prior depletion of one or more specific cell populations such as one or more or all of the following non-γδ T cell monocytes: αβ T-cells, B-cells, and NK cells, can be activated and expanded, resulting in an enriched γδ T-cell population. In some aspects, activation and expansion of γδ T-cell are performed without the presence of native or engineered APCs. In some aspects, isolation and expansion of γδ T cells can be performed using immobilized γδ T cell mitogens, including antibodies specific to activating epitopes of a γδ TCR, and other activating agents, including lectins, which bind the activating epitopes of a γδ TCR provided herein.

In certain embodiments, the isolated mixed cell population is optionally purified by, e.g., positive and/or negative selection, and contacted with one or more agents which expand γδ T-cells for about, or at least about, 2 days, about 3 days, about 4 days, about 5 days, about 6 days, about 7 days, about 8 days, about 9 days, about 10 days, about 11 days, about 12 days, about 13 days, about 14 days, about 15 days, about 17 days, about 19 days, about 21 days, about 25 days, about 29 days, about 30 days, or any range therein. For example, the isolated mixed cell population is contacted with one or more agents which expand γδ T-cells for about 1 to about 4 days, about 2 to about 4 days, about 2 to about 5 days, about 3 to about 5 days, about 5 to about 21 days, about 5 to about 19 days, about 5 to about 15 days, about 5 to about 10 days, or about 5 to about 7 days, to provide a first enriched γδ T-cell population. As another example, the isolated mixed cell population is contacted with one or more agents which expand γδ T-cells for about 7 to about 21 days, about 7 to about 19 days, about 7 to about 23 days, or about 7 to about 15 days to provide a first enriched γδ T-cell population.

In some cases, a purification or isolation step is performed between the first and second expansion steps. In some cases, the isolation step includes removal of one or more activating agents. In some cases, the isolation step includes specific isolation of γδ T-cells, or a subtype thereof. In some cases, one or more (e.g., all) activating agents (e.g., all activating agents that are not common components of cell culture media such as serum components and/or IL-2)) are removed between first and second expansion steps, but γδ T-cells are not specifically isolated from other cell types αβ T-cells).

In some embodiments, following the activation and expansion of γδ T cells using activating agents which bind to an activating epitope of a γδ TCR, in a first enrichment step, and optionally a second enrichment step, the, e.g., first, enriched γδ T cell population(s) of the invention may be further enriched or purified using techniques known in the art to obtain a second or further enriched γδ T cell population(s) in a second, third, fourth, fifth, etc. enrichment step. For example, the, e.g., first, enriched γδ T cell population(s) may be depleted of αβ T-cells, B-cells and NK cells. Positive and/or negative selection of cell surface markers expressed on the collected γδ T-cell(s) can be used to directly isolate a γδ T-cell, or a population of γδ T-cell(s) expressing similar cell surface markers from the, e.g., first, enriched γδ T-cell population(s). For instance, a γδ T-cell can be isolated from an enriched γδ T-cell population (e.g., after a first and/or second step of expansion) based on positive or negative expression of markers such as CD2, CD3, CD4, CD8, CD24, CD25, CD44, Kit, TCR α, TCR β, TCR γ (including one or more TCR γ sub-types), TCR δ (including one or more TCR δ sub-types), NKG2D, CD70, CD27, CD28, CD30, CD16, OX40, CD46, CD161, CCR7, CCR4, NKp30, NKp44, NKp46, DNAM-1, CD242, JAML, and other suitable cell surface markers.

In some embodiments, after a first step of expansion (e.g., after an isolation step performed subsequent to the first step of expansion), the expanded cells are, optionally diluted, and cultured in a second step of expansion. In preferred embodiments, the second step of expansion is performed under conditions in which culture media is replenished about every 1-2, 1-3, 1-4, 1-5, 2-5, 2-4, or 2-3 days in a second expansion step. In some embodiments, the second step of expansion is performed under conditions in which the cells are diluted or adjusted to a density that supports further γδ T-cell expansion 1, 2, 3, 4, 5, 6, or more times. In some cases, the cell density adjustment is performed contemporaneously with (i.e., on the same day as, or at the same time as) replenishment of culture media. For example, cell density can be adjusted every 1-2, 1-3, 1-4, 1-5, 2-5, 2-4, or 2-3 days in a second expansion step. Typical cell densities that support further γδ T-cell expansion include, but are not limited to, about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶ cells/mL, 10×10⁶ cells/mL, 15×10⁶ cells/mL, 20×10⁶ cells/mL, or 30×10⁶ cells/mL of culture.

In some embodiments, cell density is adjusted to a density of from about 0.5×10⁶ to about 1×10⁶ cells/mL, from about 0.5×10⁶ to about 1.5×10⁶ cells/mL, from about 0.5×10⁶ to about 2×10⁶ cells/mL, from about 0.75×10⁶ to about 1×10⁶ cells/mL, from about 0.75×10⁶ to about 1.5×10⁶ cells/mL, from about 0.75×10⁶ to about 2×10⁶ cells/mL, from about 1×10⁶ to about 2×10⁶ cells/mL, or from about 1×10⁶ to about 1.5×10⁶ cells/mL, from about 1×10⁶ to about 2×10⁶ cells/mL, from about 1×10⁶ to about 3×10⁶ cells/mL, from about 1×10⁶ to about 4×10⁶ cells/mL, from about 1×10⁶ to about 5×10⁶ cells/mL, from about 1×10⁶ to about 10×10⁶ cells/mL, from about 1×10⁶ to about 15×10⁶ cells/mL, from about 1×10⁶ to about 20×10⁶ cells/mL, or from about 1×10⁶ to about 30×10⁶ cells/mL.

In some embodiments, the second step of expansion is performed under conditions in which the cells are monitored and maintained at a predetermined cell density (or density interval) and/or maintained in culture medium having a predetermined glucose content. For example, the cells can be maintained at a viable cell density of from about 0.5×10⁶ to about 1×10⁶ cells/mL, from about 0.5×10⁶ to about 1.5×10⁶ cells/mL, from about 0.5×10⁶ to about 2×10⁶ cells/mL, from about 0.75×10⁶ to about 1×10⁶ cells/mL, from about 0.75×10⁶ to about 1.5×10⁶ cells/mL, from about 0.75×10⁶ to about 2×10⁶ cells/mL, from about 1×10⁶ to about 2×10⁶ cells/mL, or from about 1×10⁶ to about 1.5×10⁶ cells/mL, from about 1×10⁶ to about 3×10⁶ cells/mL, from about 1×10⁶ to about 4×10⁶ cells/mL, from about 1×10⁶ to about 5×10⁶ cells/mL, from about 1×10⁶ to about 10×10⁶ cells/mL, from about 1×10⁶ to about 15×10⁶ cells/mL, from about 1×10⁶ to about 20×10⁶ cells/mL, from about 1×10⁶ to about 30×10⁶ cells/mL.

In some cases, the cells can be maintained at a higher concentration for at least a portion of the expansion. For example, for a first portion of a first or second expansion, cells viability may be enhanced at a higher cell concentration. As another example, for a final portion of a first or second expansion culture volume may be most efficiently utilized at a higher cell concentration. Thus, in some embodiments, cells can be maintained at a viable cell density of from about 1×10⁶ cells/mL to about 20×10⁶ cells/mL for at least a portion of a first or second expansion culture or all of a first or second expansion culture.

As another example, the cells can be maintained in culture medium having a glucose content of from about 0.5 g/L to about 1 g/L, from about 0.5 g/L to about 1.5 g/L, from about 0.5 g/L to about 2 g/L, from about 0.75 g/L to about 1 g/L, from about 0.75 g/L to about 1.5 g/L, from about 0.75 g/L to about 2 g/L, from about 1 g/L to about 1.5 g/L, from about 1 g/L to about 2 g/L, from 1 g/L to 3 g/L, or from 1 g/L to 4 g/L. In some embodiments, the cells can be maintained in culture medium having a glucose content of about 1.25 g/L. In some cases, such as where a high cell density culture is maintained, cells can be maintained in culture medium having a glucose content of about 1 g/L to about 5 g/L, from about 1 g/L to about 4 g/L, from about 2 g/L to about 5 g/L, or from about 2 g/L to about 4 g/L.

Typically glucose content is maintained by addition of fresh serum containing or serum free culture medium to the culture. In some embodiments, the cells can be maintained at a predetermined viable cell density interval and in a culture medium having a predetermined glucose content interval, e.g., by monitoring each parameter and adding fresh media to maintain the parameters within the predetermined limits. In some embodiments, glucose content is maintained by adding fresh serum containing or serum free culture medium in the culture while removing spent medium in a perfusion bioreactor while retaining the cells inside. In some embodiments, additional parameters including, without limitation, one or more of: pH, partial pressure of O₂, O₂ saturation, partial pressure of CO₂, CO₂ saturation, lactate, glutamine, glutamate, ammonium, sodium, potassium, and calcium, are monitored and/or maintained during a γδ T-cell expansion (e.g., selective γδ T-cell expansion) or during a first or second step of γδ T-cell expansion (e.g., selective γδ T-cell expansion) described herein.

A γδ T-cell subtype may be selectively expanded from an isolated complex sample or mixed cell population that is cultured in vitro by contacting the mixed cell population with one or more agents which:

i) selectively expand δ1 T-cells by specifically binding to an epitope of a δ1 TCR,

ii) selectively expand δ2 T-cells by specifically binding to an epitope of a δ2 TCR,

iii) selectively expand δ1 and δ4 T cells by specifically binding to an epitope of a δ1 and a δ4 TCR;

iv) selectively expand δ1, δ3, δ4, and δ5 T cells by specifically binding to an epitope of a δ1, δ3, δ4, and a δ5 TCR; or

v) selectively expand δ3 T cells by specifically binding to an epitope of a δ3 TCR, to provide an enriched γδ T-cell population, e.g., in a first enrichment step.

In some cases, the one or more agents specifically bind to a δ1J2, or δ1J3 TCR, or two thereof, or all thereof. In some embodiments, γδ cells in a whole PBMC population, without prior depletion of specific cell populations such as monocytes, αβ T-cells, B-cells, and NK cells, can be activated and expanded, resulting in an enriched γδ T-cell population. In some aspects, activation and expansion of γδ T-cell are performed without the presence of native or engineered APCs. In some aspects, isolation and expansion of γδ T cells from tumor specimens can be performed using immobilized γδ T cell mitogens, including antibodies specific to activating epitopes specific of a δ1 TCR; a δ1, δ3, δ4, and δ5 TCR; a M and δ4 TCR; a δ3 TCR; or a δ2 TCR, and other activating agents, including lectins, which bind the activating epitopes specific of a δ1 TCR; a δ1, δ3, δ4 and δ5 TCR; a M and δ4 TCR; a δ3 TCR; or a δ2 TCR provided herein.

In certain embodiments, the isolated mixed cell population is contacted with one or more agents which selectively expand δ1, M and δ4, δ2, δ3, δ1 and δ2, or δ1, δ2 and δ3 T-cells for about 5 days, 6 days, about 7 days, about 8 days, about 9 days, about 10 days, about 11 days, about 12 days, about 13 days, about 14 days, about 15 days, or any range therein. For example, the isolated mixed cell population is contacted with one or more agents which selectively expand M or δ2 T-cells for about 1 to about 3 days, about 1 to about 4 days, about 1 to about 5 days, about 2 to about 3 days, about 2 to about 4 days, about 2 to about 5 days, about 3 to about 4 days, about 3 to about 5 days, about 4 to about 5 days, about 5 to about 15 days, or about 5 to about 7 days, to provide a first enriched γδ T-cell population. In some embodiments selectively expanded δ1, δ1 and δ3, M and δ4, δ2, δ3, M and δ2, or δ1, δ2 and δ3 T-cells are further expanded in a second step of expansion as described herein.

In certain embodiments, the starting isolated mixed cell population, e.g., peripheral blood sample, comprises T lymphocytes in the range of about 20-80%. In certain embodiments, the percent of residual αβ T cells and NK cells in enriched γδ T-cell population(s) of the invention is about, or less than about, 2.5% and 1%, respectively. In certain embodiments, the percent of residual αβ T cells or NK cells in enriched γδ T-cell population(s) of the invention is about, or less than about, 1%, 0.5%, 0.4%, 0.2%, 0.1%, or 0.01%. In certain embodiments, the percent of residual αβ T cells in enriched γδ T-cell population(s) of the invention is about, or less than about, 0.4%, 0.2%, 0.1%, or 0.01% (e.g., after a step of positive selection for γδ T-cells or a sub-type thereof or after depletion of αβ T cells). In some embodiments, αβ T cells are depleted, but NK cells are not depleted before or after a first and/or second γδ T-cell expansion. In certain aspects, the isolated mixed cell population is derived from a single donor. In other aspects, the isolated mixed cell population is derived from more than one donor or multiple donors (e.g., 2, 3, 4, 5, or from 2-5, 2-10, or 5-10 donors, or more).

As such, in some embodiments, the methods of the present invention can provide a clinically relevant number (>10⁸, >10⁹, >10¹⁰>10¹¹, or >10¹², or from about 10⁸ to about 10¹²) of expanded γδ T-cells from as few as one donor. In some cases, the methods of the present invention can provide a clinically relevant number (>10⁸, >10⁹, >10¹⁰, >10¹¹, or >10¹², or from about 10⁸ to about 10¹²) of expanded γδ T-cells within less than 19 or 21 days from the time of obtaining a donor sample.

Following the specific activation and expansion of the specific γδ T cell subsets using activating agents which bind to an activating epitope specific of a δ1, a δ1 and δ3 TCR, a M and δ4 TCR, or a δ2 TCR, in a first enrichment step, the first enriched γδ T cell population(s) of the invention may be further enriched or purified using techniques known in the art to obtain a second or further enriched γδ T cell population(s) in a second, third, fourth, fifth, etc. enrichment step. For example, the first enriched γδ T cell population(s) may be depleted of αβ T-cells, B-cells and NK cells. Positive and/or negative selection of cell surface markers expressed on the collected γδ T-cell(s) can be used to directly isolate a γδ T-cell, or a population of γδ T-cell(s) expressing similar cell surface markers from the first enriched γδ T-cell population(s). For instance, a γδ T-cell can be isolated from a first enriched γδ T-cell population based on positive or negative expression of markers such as CD2, CD3, CD4, CD8, CD24, CD25, CD44, Kit, TCR α, TCR β, TCR γ (or one or more subtypes thereof), TCR δ (or one or more subtypes thereof), NKG2D, CD70, CD27, CD28, CD30, CD16, OX40, CD46, CD161, CCR7, CCR4, DNAM-1, JAML, and other suitable cell surface markers.

In some embodiments, following the first γδ T-cell expansion, first enrichment step, second γδ T-cell expansion, and/or second enrichment step, of the invention, the enriched γδ T-cell population comprises clinically-relevant levels of γδ T-cell subsets of >10⁸ cells, e.g., in a culture volume of less than 10 mL, 25 mL, 50 mL, 100 mL, 150 mL, 200 mL, 500 mL, 750 mL, 1 L, 2 L, 3 L, 4 L, 5 L, 10 L, 20 L, or 25 L. For example, the methods of the present invention can provide clinically-relevant levels of γδ T-cell subsets of >10⁸ cells in a expansion culture having a volume of from 10-100 mL; from 25-100 mL; from 50-100 mL; from 75-100 mL; from 10-150 mL; from 25-150 mL; from 50-150 mL; from 75-150 mL; from 100-150 mL; from 10-200 mL; from 25-200 mL; from 50-200 mL; from 75-200 mL, from 100-200 mL; from 10-250 mL; from 25-250 mL; from 50-250 mL; from 75-250 mL, from 100-250 mL; from 150-250 mL; from 5-1,000 mL; from 10-1,000 mL, or from 100-1,000 mL; from 150-1,000 mL; from 200-1,000 mL; from 250-1,000 mL, 400 mL to 1 L, 1 L to 2 L, 2 L to 5 L, 2 L to 10 L, 4 L to 10 L, 4 L to 15 L, 4 L to 20 L, or 4 L to 25 L. In other embodiments, following the second, third, fourth, fifth, etc. enrichment step of the invention, the enriched γδ T-cell population comprises clinically-relevant levels of γδ T-cell subsets of >10⁸.

In some embodiments, γδ T-cell(s) can rapidly expand in response to contact with one or more antigens. Some γδ T-cell(s), such as Vγ9Vδ2⁺ γδ T-cell(s) rapidly expand in vitro in response to contact with some antigens, like prenyl-pyrophosphates, alkyl amines, and metabolites or microbial extracts during tissue culture. In addition, some wild-type γδ T-cell(s), such as Vγ2Vδ2⁺ γδ T-cell(s) rapidly expand in vivo in humans in response to certain types of vaccination(s). Stimulated γδ T-cells can exhibit numerous antigen-presentation, co-stimulation, and adhesion molecules that can facilitate the isolation of a γδ T-cell(s) from a complex sample. A γδ T-cell(s) within a complex sample can be stimulated in vitro with at least one antigen for 1 day, 2 days, 3 days, 4 days, 5 days, 6 days, 7 days, about 5-15 days, 5-10 days, or 5-7 days, or another suitable period of time, e.g., in combination with, before, or after expansion with a selective γδ T-cell expansion agent described herein such as an antibody or an immobilized antibody. Stimulation of the γδ T-cell with a suitable antigen can expand the γδ T-cell population in vivo by administration of one or more suitable agents to a subject, or in vitro.

Non-limiting examples of antigens that may be used to stimulate the expansion of γδ T-cell(s) from a complex sample in vitro include, prenyl-pyrophosphates, such as isopentenyl pyrophosphate (IPP), alkyl-amines, metabolites of human microbial pathogens, metabolites of commensal bacteria, -methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine and nitrogen containing bisphosphonates (e.g., aminophosphonates).

Activation and expansion of γδ T-cells can be performed using activation and co-stimulatory agents described herein to trigger specific γδ T-cell proliferation and persistent populations. In some embodiments, activation and expansion of γδ T-cells from different cultures can achieve distinct clonal or mixed polyclonal population subsets. In some embodiments, different agonist agents can be used to identify agents that provide specific γδ activating signals. In one aspect, agents that provide specific γδ activating signals can be different monoclonal antibodies (MAbs) directed against the γδ TCRs.

In one aspect, the MAbs can bind to different epitopes on the constant or variable regions of γ TCR and/or δ TCR. In one aspect, the MAbs can include γδ TCR pan MAbs. In one aspect, the γδ TCR pan MAbs may recognize domains shared by different γ and δ TCRs on either the γ or δ chain or both, including 03 cell populations. In one aspect, the antibodies may be 5A6.E9 (Thermo scientific), B1 (Biolegend), IMMU510 and/or 11F2 (11F2) (Beckman Coulter). In one aspect, the MAbs can be directed to specific domains unique to the variable regions of the γ chain (7A5 Mab, directed to like Vγ9 TCR (Thermo Scientific #TCR1720)), or domains on Vδ1 variable region (Mab TS8.2 (Thermo scientific #TCR1730; MAb TS-1 (ThermoFisher #TCR 1055), MAb R9.12 (Beckman Coulter #IM1761)), or Vδ2 chain (MAb 15D (Thermo Scientific #TCR1732 or Life technologies #TCR2732) B6 (Biolegend #331402), one or more of the δ1-#antibodies described in FIGS. 1-2, one or more of the δ2-#antibodies described in FIGS. 3-4, or one or more of δ3-08, δ3-20, 63-23, δ3-31, δ3-42, δ3-47 and δ3-58 described in FIG. 5.

In some embodiments, antibodies against different domains of the γδ TCR (pan antibodies and antibodies recognizing specific variable region epitopes on subset populations) can be combined to evaluate their ability to enhance activation of γδ T cells. In some embodiments, γδ T-cells activators can include γδ TCR-binding agents such as MICA, an agonist antibody to NKG2D, an, e.g., Fc tag, fusion protein of MICA, ULBP1, or ULBP3 (R&D systems Minneapolis, Minn.) ULBP2, or ULBP6 (Sino Biological Beijing, China). In some embodiments, companion co-stimulatory agents to assist in triggering specific γδ T cell proliferation without induction of cell anergy and apoptosis can be identified. These co-stimulatory agents can include ligands to receptors expressed on γδ cells, such as ligand(s) to one or more of the following: NKG2D, CD161, CD70, JAML, DNAX, CD81 accessory molecule-1 (DNAM-1) ICOS, CD27, CD196, CD137, CD30, HVEM, SLAM, CD122, DAP, and CD28. In some aspects, co-stimulatory agents can be antibodies specific to unique epitopes on CD2 and CD3 molecules. CD2 and CD3 can have different conformation structures when expressed on αβ or γδ T-cells (s), and in some cases, specific antibodies to CD3 and CD2 can lead to selective activation of γδ T-cells.

A population of γδ T-cell(s) may be expanded ex vivo prior to engineering of the γδ T-cell(s). Non-limiting example of reagents that can be used to facilitate the expansion of a γδ T-cell population in vitro include anti-CD3 or anti-CD2, anti-CD27, anti-CD30, anti-CD70, anti-OX40 antibodies, IL-2, IL-4, IL-7, IL-9, IL-12, IL-15, IL-18, IL-19, IL-21, IL 23, IL-33, IFNγ, granulocyte-macrophage colony stimulating factor (GM-CSF), granulocyte colony stimulating factor (G-CSF), CD70 (CD27 ligand), concavalin A (ConA), pokeweed (PWM), protein peanut agglutinin (PNA), soybean agglutinin (SBA), Les Culinaris Agglutinin (LCA), Pisum Sativum Agglutinin (PSA), Helix pomatia agglutinin (HPA), Vicia graminea Lectin (VGA), Phaseolus Vulgaris Erythroagglutinin (PHA-E), Phaseolus Vulgaris Leucoagglutinin (PHA-L), Sambucus Nigra Lectin (SNA, EBL), Maackia Amurensis, Lectin II (MAL II), Sophora Japonica Agglutinin (SJA), Dolichos Biflorus Agglutinin (DBA), Lens Culinaris Agglutinin (LCA), Wisteria Floribunda Lectin (WFA, WFL) or another suitable mitogen capable of stimulating T-cell proliferation.

Genetic engineering of the γδ T-cell(s) may comprise stably integrating a construct expressing a tumor recognition moiety, such as an αβ TCR, a γδ TCR, a CAR encoding an antibody, an antigen binding fragment thereof, or a lymphocyte activation domain into the genome of the isolated γδ T-cell(s), a cytokine (e.g., IL-15, IL-12, IL-2, IL-7, IL-21, IL-18, IL-19, IL-33, IL-4, IL-9, IL-23, or IL1β) to enhance T-cell proliferation, survival, and function ex vivo and in vivo. In some cases, the cytokine is IL-2, IL-15, IL-12, or IL-21. In some cases, the cytokine is IL-2. In some cases, the cytokine is IL-15. In some cases, the cytokine is IL-4. In some cases, the cytokine is a common gamma chain cytokine selected from the group consisting of IL-2, IL-4, IL-7, IL-9, IL-15, and IL-21, or a combination thereof. Genetic engineering of the isolated γδ T-cell may also comprise deleting or disrupting gene expression from one or more endogenous genes in the genome the isolated γδ T-cell, such as the MHC locus (loci).

Ex-Vivo Expansion of γδ T-Cells

In other aspects, the present disclosure provides methods for the ex vivo expansion of a population of non-engineered and engineered γδ T-cells for adoptive transfer therapy. A non-engineered or engineered γδ T-cell of the disclosure may be expanded ex vivo. The ex vivo expansion can be performed after an in vivo expansion, isolation, and optional purification. The ex vivo expansion can additionally or alternatively be performed before in vivo expansion to provide a γδ T-cell population that is then administered to a subject and subject to one or more in vivo expansion or maintenance methods described herein. The ex vivo expansion can be performed with a mixed cell population by, e.g., directly contacting an isolated sample containing γδ T-cell with one or more agents that selectively expand γδ T-cells. Additionally or alternatively, the ex vivo expansion can be performed after positive selection for γδ T-cells or one or more sub-types thereof, and/or negative selection to remove one or more of αβ T cells, B cells, or NK cells.

A non-engineered or engineered γδ T-cell of the disclosure can be expanded in vitro without activation by APCs, or without co-culture with APCs and/or aminophosphonates. Additionally, or alternatively, a non-engineered or engineered γδ T-cell of the disclosure can be expanded in vitro with at least one expansion step that includes activation by or co-culture with APCs and/or with one or more aminophosphonates.

In some embodiments, a non-engineered or engineered γδ T-cell of the disclosure can be expanded in vitro without activation by APC in a first γδ T-cell expansion, and then expanded in vitro with activation by APC in a second γδ T-cell expansion. In some cases, the first γδ T-cell expansion includes contacting the γδ T-cells with one or more agents which (a) expand γδ T-cells, or (b) selectively expand δ1 T-cells; δ2 T-cells; δ1 T-cells and δ3 T-cells; δ1 T-cells and δ4 T-cells; or δ1, δ3, δ4, and δ5 T-cells by binding to an activating epitope specific of a δ1 TCR; a δ2 TCR; a δ1 and δ4 TCR; or a δ1, δ3, δ4, and δ5 TCR respectively.

In some cases, the second γδ T-cell expansion is performed in a culture medium that is free of the one or more agents used in the first γδ T-cell expansion. In some cases, the second γδ T-cell expansion is performed in a culture medium that contains one or more second agents that (a) expand T cells, (b) expand γδ T-cells, or (c) selectively expand δ1 T-cells; δ2 T-cells; δ1 T-cells and δ3 T-cells; δ1 T-cells and δ4 T-cells; or δ1, δ3, δ4, and δ5 T-cells by binding to an activating epitope specific of a δ1 TCR; a δ2 TCR; a δ1 and δ4 TCR; or a δ1, δ3, δ4, and δ5 TCR respectively.

In some cases, the second agents are different (e.g., have a different primary amino acid sequence and/or bind a structurally different γδ TCR epitope) as compared to the agents used in the first γδ T-cell expansion. In some cases, the second agents bind an overlapping γδ TCR epitope, the same γδ TCR epitope, or can compete for binding to γδ TCR with the agents used in the first γδ T-cell expansion. In some cases, the second agents are expressed on the cell surface of an APC. In some cases, the second agents are bound to the surface of an APC, e.g., by a binding interaction between a constant region of the second agent and an Fc-receptor on the surface of the APC. In some cases, the second agents are soluble. In some cases, the second γδ T-cell expansion is performed in a culture medium containing soluble second agents and APCs, optionally wherein the APC express on their cell surface or bind to their cell surface an agent that expands or selectively expands a γδ T cell population.

In some cases, the first γδ T-cell expansion is performed without an APC, and the second γδ T-cell expansion is performed with an APC. In some cases, the second γδ T-cell expansion is performed with an APC and one or more second agents that (a) expand T cells, (b) expand γδ T-cells, or (c) selectively expand δ1 T-cells; δ2 T-cells; δ1 T-cells and δ3 T-cells; δ1 T-cells and δ4 T-cells; or δ1, δ3, δ4, and δ5 T-cells by binding to an activating epitope specific of a δ1 TCR; a δ2 TCR; a δ1 and δ4 TCR; or a δ1, δ3, δ4, and δ5 TCR respectively.

One of skill in the art will appreciate that, in certain embodiments, the methods of the second expansion step described herein can be performed as a first expansion step and methods of the first step described herein can be performed as a second expansion step. As an example, and without limitation, in some embodiments, a mixed population of cells (e.g., PBMC) can be expanded by contacting with an APC in a first step, and then expanded in the absence of an APC, e.g., by contacting the expanded population from the first expansion step with an immobilized agent that selectively expands δ1 T-cells; δ2 T-cells; δ1 T-cells and δ3 T-cells; δ1 T-cells and δ4 T-cells; or δ1, δ3, δ4, and δ5 T-cells by binding to an activating epitope specific of a δ1 TCR; a δ2 TCR; a δ1 and δ4 TCR; or a δ1, δ3, δ4, and δ5 TCR respectively.

A method of the invention can expand various γδ T-cell(s) populations, such as a Vγ1⁺, a Vγ2⁺, or Vγ3⁺ γδ T-cell population. In some cases, a method of the invention can expand a Vδ1⁺ T-cell population; a Vδ1⁺ and a Vδ3⁺ T-cell population; a Vδ1⁺ and a Vδ4⁺ T-cell population; a Vδ1⁺ and a Vδ2⁺ T-cell population; or a Vδ1⁺, Vδ3⁺, Vδ4⁺, and a Vδ5⁺ T-cell population.

In some instances, a γδ T-cell population can be expanded in vitro in fewer than 36 days, fewer than 35 days, fewer than 34 days, fewer than 33 days, fewer than 32 days, fewer than 31 days, fewer than 30 days, fewer than 29 days, fewer than 28 days, fewer than 27 days, fewer than 26 days, fewer than 25 days, fewer than 24 days, fewer than 23 days, fewer than 22 days, fewer than 21 days, fewer than 20 days, fewer than 19 days, fewer than 18 days, fewer than 17 days, fewer than 16 days, fewer than 15 days, fewer than 14 days, fewer than 13 days, fewer than 12 days, fewer than 11 days, fewer than 10 days, fewer than 9 days, fewer than 8 days, fewer than 7 days, fewer than 6 days, fewer than 5 days, fewer than 4 days, or fewer than 3 days.

In some aspects, provided are methods for selectively expanding various γδ T-cells, including engineered and non-engineered γδ T-cells by contacting the γδ T-cells from the mixed cell population with an activation agent. In some cases, the activation or activating agent binds to a specific epitope on a cell-surface receptor of a γδ T-cell. The activation agent can be an antibody, such as a monoclonal antibody. The activation agent can specifically activate the growth of one or more types of γδ T-cells, such 61, 62, 61 and 63, or 61 and δ4 cell populations. In some embodiments the activation agent specifically activates the growth of δ1 cell populations to provide an enriched 61T-cell population. In other cases, the activation agent specifically activates the growth of δ2 cell populations to provide an enriched δ2 T-cell population. In other cases, the activation agent specifically activates the growth of δ3 cell populations to provide an enriched δ3 T-cell population.

An activation agent may stimulate the expansion of engineered and non-engineered γδ T-cells at a fast rate of growth. For instance, an agent that stimulates an expansion of the γδ T-cell population at a mean rate of 1 cell division in less than 30 hours, 1 cell division in less than 29 hours, 1 cell division in less than 28 hours, 1 cell division in less than 27 hours, 1 cell division in less than 26 hours, 1 cell division in less than 25 hours, 1 cell division in less than 24 hours, 1 cell division in less than 23 hours, 1 cell division in less than 22 hours, 1 cell division in less than 21 hours, 1 cell division in less than 20 hours, 1 cell division in less than 19 hours, 1 cell division in less than 18 hours, 1 cell division in less than 17 hours, 1 cell division in less than 16 hours, 1 cell division in less than 15 hours, 1 cell division in less than 14 hours, 1 cell division in less than 13 hours, 1 cell division in less than 12 hours, 1 cell division in less than 11 hours, 1 cell division in less than 10 hours, 1 cell division in less than 9 hours, 1 cell division in less than 8 hours, 1 cell division in less than 7 hours, 1 cell division in less than 6 hours, 1 cell division in less than 5 hours, 1 cell division in less than 4 hours, 1 cell division in less than 3 hours, 1 cell division in less than 2 hours.

In some cases, an activation agent may stimulate the expansion of engineered and non-engineered γδ T-cells at a mean rate of about 1 division per about 4 hours, a mean rate of about 1 division per about 5 hours, a mean rate of about 1 division per about 6 hours, a mean rate of about 1 division per about 7 hours, a mean rate of about 1 division per about 8 hours, a mean rate of about 1 division per about 9 hours, a mean rate of about 1 division per about 10 hours, a mean rate of about 1 division per about 11 hours, a mean rate of about 1 division per about 12 hours, a mean rate of about 1 division per about 13 hours, a mean rate of about 1 division per about 14 hours, a mean rate of about 1 division per about 15 hours, a mean rate of about 1 division per about 16 hours, a mean rate of about 1 division per about 17 hours, a mean rate of about 1 division per about 18 hours, a mean rate of about 1 division per about 19 hours, a mean rate of about 1 division per about 20 hours, a mean rate of about 1 division per about 21 hours, a rate of about 1 division per about 22 hours, a rate of about 1 division per about 23 hours, a mean rate of about 1 division per about 24 hours, a mean rate of about 1 division per about 25 hours, a mean rate of about 1 division per about 26 hours, a mean rate of about 1 division per about 27 hours, a rate of about 1 division per about 28 hours, a rate of about 1 division per about 29 hours, a mean rate of about 1 division per about 30 hours, a mean rate of about 1 division per about 31 hours, a mean rate of about 1 division per about 32 hours, a mean rate of about 1 division per about 33 hours, a rate of about 1 division per about 34 hours, a rate of about 1 division per about 35 hours, a mean rate of about 1 division per about 36 hours.

In some cases, an activation agent may stimulate the rapid expansion of engineered and/or non-engineered γδ T-cells in a γδ T-cell expansion culture, wherein the rapid expansion is at any one of the foregoing mean rates of cell division and is maintained for between about 1 contiguous day and about 19 contiguous days, between about 1 contiguous day and about 14 contiguous days, between about 1 contiguous day and about 7 contiguous days, between about 1 contiguous day and about 5 contiguous days, between about 2 contiguous days and about 19 contiguous days, between about 2 contiguous days and about 14 contiguous days, between about 2 contiguous days and about 7 contiguous days, between about 2 contiguous days and about 5 contiguous days, between about 4 contiguous days and about 19 contiguous days, between about 4 contiguous days and about 14 contiguous days, between about 4 contiguous days and about 7 contiguous days, or between about 4 contiguous days and about 5 contiguous days.

In some cases, an activation agent may stimulate the expansion of engineered and/or non-engineered γδ T-cells in a γδ T-cell expansion culture that has been maintained for between about 2 and about 7 contiguous days, or between about 2 and about 5 contiguous days, at a mean rate of about 1 division per about 12 hours (e.g., 10-12 hours), a mean rate of about 1 division per about 13 hours (e.g., 10-13 hours), a mean rate of about 1 division per about 14 hours (e.g., 10-14 hours), a mean rate of about 1 division per about 15 hours (e.g., 10-15 hours), a mean rate of about 1 division per about 16 hours (e.g., 10-16 hours), a mean rate of about 1 division per about 17 hours (e.g., 10-17 hours or 12-17 hours), a mean rate of about 1 division per about 18 hours (e.g., 10-18 hours or 12-18 hours), a mean rate of about 1 division per about 19 hours (e.g., 10-19 hours or 12-19 hours), a mean rate of about 1 division per about 20 hours (e.g., 12-20 hours, 16-20 hours or 18-20 hours), a mean rate of about 1 division per about 21 hours (e.g., 12-21 hours, 16-21 hours or 18-21 hours), a rate of about 1 division per about 22 hours (e.g., 12-22 hours, 16-22 hours or 18-22 hours), a rate of about 1 division per about 23 hours or less (e.g., 12-23 hours, 16-23 hours or 18-23 hours), a mean rate of about 1 division per about 24 hours (e.g., 12-24 hours, 16-24 hours or 18-24 hours).

In some cases, an activation agent may stimulate the expansion of engineered and/or non-engineered γδ T-cells in a γδ T-cell expansion culture that has been maintained for between about 2 and about 7 contiguous days, or between about 2 and about 5 contiguous days at a mean rate of about 1 division per about 25 hours (e.g., 12-25 hours, 16-25 hours 18-25 hours, or 20-25 hours), a mean rate of about 1 division per about 26 hours (e.g., 12-26 hours, 16-26 hours 18-26 hours, or 20-26 hours), a mean rate of about 1 division per about 27 hours (e.g., 12-27 hours, 16-27 hours 18-27 hours, or 20-27 hours), a rate of about 1 division per about 28 hours (e.g., 12-28 hours, 16-28 hours 18-28 hours, 20-28 hours, or 22-28 hours), a rate of about 1 division per about 29 hours (e.g., 16-29 hours 18-29 hours, 20-29 hours, or 22-29 hours), a mean rate of about 1 division per about 30 hours (e.g., 16-30 hours 18-30 hours, 20-30 hours, or 22-30 hours), a mean rate of about 1 division per about 31 hours (e.g., 16-31 hours 18-31 hours, 20-31 hours, 22-31 hours, or 24-31 hours), a mean rate of about 1 division per about 32 hours (e.g., 18-32 hours, 20-32 hours, 22-32 hours, or 24-32 hours), a mean rate of about 1 division per about 33 hours (e.g., 18-33 hours, 20-33 hours, 22-33 hours, or 24-33 hours), a rate of about 1 division per about 34 hours (e.g., 18-34 hours, 20-34 hours, 22-34 hours, or 24-34 hours), a rate of about 1 division per about 35 hours (e.g., 18-35 hours, 20-35 hours, 22-35 hours, or 24-35 hours), a mean rate of about 1 division per about 36 hours (e.g., 18-36 hours, 20-36 hours, 22-36 hours, or 24-36 hours).

In some cases, an activation agent may stimulate the expansion of engineered and/or non-engineered γδ T-cells in a γδ T-cell expansion culture that has been maintained for at least 14 contiguous days at a mean rate of about 1 division per about 12 hours (e.g., 10-12 hours), a mean rate of about 1 division per about 13 hours (e.g., 10-13 hours), a mean rate of about 1 division per about 14 hours (e.g., 10-14 hours), a mean rate of about 1 division per about 15 hours (e.g., 10-15 hours), a mean rate of about 1 division per about 16 hours (e.g., 10-16 hours), a mean rate of about 1 division per about 17 hours (e.g., 10-17 hours or 12-17 hours), a mean rate of about 1 division per about 18 hours (e.g., 10-18 hours or 12-18 hours), a mean rate of about 1 division per about 19 hours (e.g., 10-19 hours or 12-19 hours), a mean rate of about 1 division per about 20 hours (e.g., 12-20 hours, 16-20 hours or 18-20 hours), a mean rate of about 1 division per about 21 hours (e.g., 12-21 hours, 16-21 hours or 18-21 hours), a rate of about 1 division per about 22 hours (e.g., 12-22 hours, 16-22 hours or 18-22 hours), a rate of about 1 division per about 23 hours or less (e.g., 12-23 hours, 16-23 hours or 18-23 hours), a mean rate of about 1 division per about 24 hours (e.g., 12-24 hours, 16-24 hours or 18-24 hours).

In some cases, an activation agent may stimulate the expansion of engineered and/or non-engineered γδ T-cells in a γδ T-cell expansion culture that has been maintained for at least 14 contiguous days at a mean rate of about 1 division per about 25 hours (e.g., 12-25 hours, 16-25 hours 18-25 hours, or 20-25 hours), a mean rate of about 1 division per about 26 hours (e.g., 12-26 hours, 16-26 hours 18-26 hours, or 20-26 hours), a mean rate of about 1 division per about 27 hours (e.g., 12-27 hours, 16-27 hours 18-27 hours, or 20-27 hours), a rate of about 1 division per about 28 hours (e.g., 12-28 hours, 16-28 hours 18-28 hours, 20-28 hours, or 22-28 hours), a rate of about 1 division per about 29 hours (e.g., 16-29 hours 18-29 hours, 20-29 hours, or 22-29 hours), a mean rate of about 1 division per about 30 hours (e.g., 16-30 hours 18-30 hours, 20-30 hours, or 22-30 hours), a mean rate of about 1 division per about 31 hours (e.g., 16-31 hours 18-31 hours, 20-31 hours, 22-31 hours, or 24-31 hours), a mean rate of about 1 division per about 32 hours (e.g., 18-32 hours, 20-32 hours, 22-32 hours, or 24-32 hours), a mean rate of about 1 division per about 33 hours (e.g., 18-33 hours, 20-33 hours, 22-33 hours, or 24-33 hours), a rate of about 1 division per about 34 hours (e.g., 18-34 hours, 20-34 hours, 22-34 hours, or 24-34 hours), a rate of about 1 division per about 35 hours (e.g., 18-35 hours, 20-35 hours, 22-35 hours, or 24-35 hours), a mean rate of about 1 division per about 36 hours (e.g., 18-36 hours, 20-36 hours, 22-36 hours, or 24-36 hours).

In some cases, an activation agent may stimulate the expansion of engineered and/or non-engineered γδ T-cells in a γδ T-cell expansion culture that has been maintained for at least 19 contiguous days at a mean rate of about 1 division per about 12 hours (e.g., 10-12 hours), a mean rate of about 1 division per about 13 hours (e.g., 10-13 hours), a mean rate of about 1 division per about 14 hours (e.g., 10-14 hours), a mean rate of about 1 division per about 15 hours (e.g., 10-15 hours), a mean rate of about 1 division per about 16 hours (e.g., 10-16 hours), a mean rate of about 1 division per about 17 hours (e.g., 10-17 hours or 12-17 hours), a mean rate of about 1 division per about 18 hours (e.g., 10-18 hours or 12-18 hours), a mean rate of about 1 division per about 19 hours (e.g., 10-19 hours or 12-19 hours), a mean rate of about 1 division per about 20 hours (e.g., 12-20 hours, 16-20 hours or 18-20 hours), a mean rate of about 1 division per about 21 hours (e.g., 12-21 hours, 16-21 hours or 18-21 hours), a rate of about 1 division per about 22 hours (e.g., 12-22 hours, 16-22 hours or 18-22 hours), a rate of about 1 division per about 23 hours or less (e.g., 12-23 hours, 16-23 hours or 18-23 hours), a mean rate of about 1 division per about 24 hours (e.g., 12-24 hours, 16-24 hours or 18-24 hours).

In some cases, an activation agent may stimulate the expansion of engineered and/or non-engineered γδ T-cells in a γδ T-cell expansion culture that has been maintained for at least 19 contiguous days at a mean rate of about 1 division per about 25 hours (e.g., 12-25 hours, 16-25 hours 18-25 hours, or 20-25 hours), a mean rate of about 1 division per about 26 hours (e.g., 12-26 hours, 16-26 hours 18-26 hours, or 20-26 hours), a mean rate of about 1 division per about 27 hours (e.g., 12-27 hours, 16-27 hours 18-27 hours, or 20-27 hours), a rate of about 1 division per about 28 hours (e.g., 12-28 hours, 16-28 hours 18-28 hours, 20-28 hours, or 22-28 hours), a rate of about 1 division per about 29 hours (e.g., 16-29 hours 18-29 hours, 20-29 hours, or 22-29 hours), a mean rate of about 1 division per about 30 hours (e.g., 16-30 hours 18-30 hours, 20-30 hours, or 22-30 hours), a mean rate of about 1 division per about 31 hours (e.g., 16-31 hours 18-31 hours, 20-31 hours, 22-31 hours, or 24-31 hours), a mean rate of about 1 division per about 32 hours (e.g., 18-32 hours, 20-32 hours, 22-32 hours, or 24-32 hours), a mean rate of about 1 division per about 33 hours (e.g., 18-33 hours, 20-33 hours, 22-33 hours, or 24-33 hours), a rate of about 1 division per about 34 hours (e.g., 18-34 hours, 20-34 hours, 22-34 hours, or 24-34 hours), a rate of about 1 division per about 35 hours (e.g., 18-35 hours, 20-35 hours, 22-35 hours, or 24-35 hours), a mean rate of about 1 division per about 36 hours (e.g., 18-36 hours, 20-36 hours, 22-36 hours, or 24-36 hours).

An activation agent may stimulate the expansion of sub-populations of engineered or non-engineered γδ T-cells at different rates of growth. For instance, an agent may stimulate the growth of a δ1 cell population at a faster rate such that over a period of time from 1 day to 90 days of culture (e.g., about 1 day to about 19, 21, or 23 days of culture) the expansion results in greater than about 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 50,000-fold, 70,000-fold, 100,000-fold or 1,000,000-fold expansion over another γδ T-cell population, such as a 62 or 63 population; over a starting number of γδ T-cells before the expansion; over a starting number of γδ1 T-cells before the expansion; or over an αβ T cell population in the culture.

In other cases, the agent may stimulate the growth of a δ1 and δ4 population at faster rates such that over a period of time from 1 day to 90 days of culture (e.g., about 1 day to about 19, 21, or 23 days of culture) the expansion results in greater than 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 50,000-fold, 70,000-fold, 100,000-fold or 1,000,000-fold expansion over a δ2 T-cell population; over another γδ T-cell sub-population; over a starting number of γδ T-cells before the expansion; over a starting number of γδ1 T-cells before the expansion; over a starting number of γδ1 and γδ3 T-cells before the expansion; or over an αβ T cell population in the culture.

In other cases, the agent may stimulate the growth of a 61 and 64 population at faster rates such that over a period of time from 1 day to 90 days of culture (e.g., about 1 day to about 19, 21, or 23 days of culture) the expansion results in greater than 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 50,000-fold, 70,000-fold, 100,000-fold or 1,000,000-fold expansion over a δ2 T-cell population; over another γδ T-cell sub-population; over a starting number of γδ T-cells before the expansion; over a starting number of γδ1 T-cells before the expansion; over a starting number of γδ1 and γδ4 T-cells before the expansion; or over an αβ T cell population in the culture.

In other cases, the agent may stimulate the growth of a δ1, δ3, δ4 and δ5 population at faster rates such that over a period of time from 1 day to 90 days of culture (e.g., about 1 day to about 19, 21, or 23 days of culture) the expansion results in greater than 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 50,000-fold, 70,000-fold, 100,000-fold or 1,000,000-fold expansion over a δ2 T-cell population; over another γδ T-cell sub-population; over a starting number of γδ T-cells before the expansion; over a starting number of γδ1 T-cells before the expansion; over a starting number of γδ1 and γδ3 T-cells before the expansion; over a starting number of γδ1, γδ3, γδ4, and γδ5 T-cells before the expansion; or over an αβ T cell population in the culture.

In other cases, the agent may stimulate the growth of a δ2 population at faster rates such that over a period of time from 1 day to 90 days of culture (e.g., about 1 day to about 19, 21, or 23 days of culture) the expansion results in greater than 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 50,000-fold, 70,000-fold, 100,000-fold or 1,000,000-fold expansion over a δ1 T-cell population; over a δ3 T-cell population; over another γδ T-cell sub-population; over a starting number of γδ T-cells before the expansion, over a starting number of γδ2 T-cells before the expansion, or over 1:0 T-cells.

In some aspects, the disclosure provides an engineered or non-engineered γδ T-cell population, in contact with an agent that stimulates an expansion of the γδ T-cell population at a rapid rate, such as a rate of about 1 cell division per 30 hours or faster. In some cases, the agent selectively stimulates the proliferation of either δ1; δ2; δ1 and δ4; or δ1, δ3, δ4, and δ5 T-cells. A γδ T-cell population can comprise an amount of non-engineered γδ T-cells and an amount of engineered γδ T-cells. In some cases, the γδ T-cell population comprises different percentages of δ1, δ2, δ3, and δ4 T-cells. An engineered or non-engineered γδ T-cell population can comprise, for example, fewer than 90% δ1 T-cells, fewer than 80% δ1 T-cells, fewer than 70% δ1 T-cells, fewer than 60% δ1 T-cells, fewer than 50% δ1 T-cells, fewer than 40% δ1 T-cells, fewer than 30% δ1 T-cells, fewer than 20% δ1 T-cells, fewer than 10% δ1 T-cells, or fewer than 5% δ1 T-cells. Alternatively, an engineered or non-engineered γδ T-cell population can comprise greater than 5% δ1 T-cells, greater than 10% δ1 T-cells, greater than 20% δ1 T-cells, greater than 30% δ1 T-cells, greater than 40% δ1 T-cells, greater than 50% δ1 T-cells, greater than 60% δ1 T-cells, greater than 70% δ1 T-cells, greater than 80% δ1 T-cells, or greater than 90% δ1 T-cells. In some cases, the agent is one of the selective expansion agents described herein. In some cases, the agent is immobilized on a surface such as a cell culture surface, or a surface of an APC (e.g., expressed on the surface of the APC or bound to an Fc receptor expressed on the surface of the APC).

An engineered or non-engineered γδ T-cell population can comprise, for example, fewer than 90% δ2 T-cells, fewer than 80% δ2 T-cells, fewer than 70% δ2 T-cells, fewer than 60% δ2 T-cells, fewer than 50% δ2 T-cells, fewer than 40% δ2 T-cells, fewer than 30% δ2 T-cells, fewer than 20% δ2 T-cells, fewer than 10% δ2 T-cells, or fewer than 5% δ2 T-cells. Alternatively, an engineered or non-engineered γδ T-cell population can comprise greater than 5% δ2 T-cells, greater than 10% δ2 T-cells, greater than 20% δ2 T-cells, greater than 30% δ2 T-cells, greater than 40% δ2 T-cells, greater than 50% δ2 T-cells, greater than 60% δ2 T-cells, greater than 70% δ2 T-cells, greater than 80% δ2 T-cells, or greater than 90% δ2 T-cells.

An engineered or non-engineered γδ T-cell population can comprise, for example, fewer than 90% δ1 and δ4 T-cells, fewer than 80% δ1 and δ4 T-cells, fewer than 70% δ1 and δ4 T-cells, fewer than 60% δ1 and δ4 T-cells, fewer than 50% δ1 and δ4 T-cells, fewer than 40% δ1 and δ4 T-cells, fewer than 30% δ1 and δ4 T-cells, fewer than 20% δ1 and δ4 T-cells, fewer than 10% δ1 and δ4 T-cells, or fewer than 5% δ1 and δ4 T-cells. Alternatively, an engineered or non-engineered γδ T-cell population can comprise greater than 5% δ1 and δ4 T-cells, greater than 10% δ1 and δ4 T-cells, greater than 20% δ1 and δ4 T-cells, greater than 30% δ1 and δ4 T-cells, greater than 40% δ1 and 64 T-cells, greater than 50% δ1 and δ4 T-cells, greater than 60% δ1 and δ4 T-cells, greater than 70% δ1 and δ4 T-cells, greater than 80% δ1 and δ4 T-cells, or greater than 90% δ1 and δ4 T-cells.

An engineered or non-engineered γδ T-cell population can comprise, for example, fewer than 90% δ4 T-cells, fewer than 80% δ4 T-cells, fewer than 70% δ4 T-cells, fewer than 60% δ4 T-cells, fewer than 50% δ4 T-cells, fewer than 40% δ4 T-cells, fewer than 30% δ4 T-cells, fewer than 20% δ4 T-cells, fewer than 10% δ4 T-cells, or fewer than 5% δ4 T-cells. Alternatively, an engineered or non-engineered γδ T-cell population can comprise greater than 5% δ1 and δ4 T-cells, greater than 10% δ1 and δ4 T-cells, greater than 20% δ1 and δ4 T-cells, greater than 30% δ1 and δ4 T-cells, greater than 40% δ1 and δ4 T-cells, greater than 50% δ1 and δ4 T-cells, greater than 60% δ1 and 64 T-cells, greater than 70% δ1 and δ4 T-cells, greater than 80% δ1 and δ4 T-cells, or greater than 90% 61 and δ4 T-cells. An engineered or non-engineered γδ T-cell population can comprise, for example, fewer than 90% δ1 and δ4 T-cells, fewer than 80% δ1 and δ4 T-cells, fewer than 70% δ1 and δ4 T-cells, fewer than 60% δ1 and δ4 T-cells, fewer than 50% δ1 and δ4 T-cells, fewer than 40% δ1 and δ4 T-cells, fewer than 30% δ1 and δ4 T-cells, fewer than 20% δ1 and δ4 T-cells, fewer than 10% δ1 and δ4 T-cells, or fewer than 5% δ1 and δ4 T-cells.

In certain embodiments, the present invention provides admixtures of expanded γδ T-cell populations comprising 10-90% M T-cells and 90-10% δ2 T-cells. In certain embodiments, the present invention provides admixtures of expanded γδ T-cell populations comprising 10-90% M and δ3 T-cells and 90-10% δ2 T-cells. In certain embodiments, the present invention provides admixtures of expanded γδ T-cell populations comprising 10-90% δ1 and δ4 T-cells and 90-10% δ2 T-cells. In certain embodiments, the present invention provides admixtures of expanded γδ T-cell populations comprising 10-90% δ1, δ3, δ4 and δ5 T-cells and 90-10% δ2 T-cells.

One or more activation agent can contact the γδ T-cells (for example an activator γδ T cell innate receptor) and thereafter a costimulatory molecule can contact the γδ T-cells to provide further stimulation and to expand the γδ T-cells. In some embodiments, the activation agent and/or costimulatory agent can be lectins of plant and non-plant origin; monoclonal antibodies that activate γδ T-cells, and other non-lectin/non-antibody agents. In other cases, the plant lectin can be concanavalin A (conA) although other plant lectins such as may be used. Other examples of lectins include protein peanut agglutinin (PNA), soybean agglutinin (SBA), les culinaris agglutinin (LCA), Pisum sativum agglutinin (PSA), Helix pomatia agglutinin (HPA), Vicia graminea Lectin (VGA), Phaseolus Vulgaris Erythroagglutinin (PHA-E), Phaseolus Vulgaris Leucoagglutinin (PHA-L), Sambucus Nigra Lectin (SNA, EBL), Maackia Amurensis, Lectin II (MAL II), Sophora Japonica Agglutinin (SJA), Dolichos Biflorus Agglutinin (DBA), Lens Culinaris Agglutinin (LCA), Wisteria Floribunda Lectin (WFA, WFL).

Non-limiting examples of activating agents and costimulatory molecules include any one or more antibodies selective for a δ or γ-chain or subtypes thereof described herein, antibodies such as 5A6.E9, B1, TS8.2, 15D, B6, B3, TS-1, γ3.20, 7A5, IMMU510, R9.12, 11F2, or a combination thereof. Other examples of activating agents and costimulatory molecules include zoledronate, phorbol 12-myristate-13-acetate (TPA), mezerein, staphylococcal enterotoxin A (SEA), streptococcal protein A, or a combination thereof.

In other cases, the activation agent and/or costimulatory agent can be, antibodies or ligands to TCR, TCR, γ TCR, δ TCR, CD277, CD28, CD46, CD81, CTLA4, ICOS, PD-1, CD30, NKG2D, NKG2A, HVEM, 4-1 BB (CD137), OX40 (CD134), CD70, CD80, CD86, DAP, CD122, GITR, FcεRIγ, CD1, CD16, CD161, DNAX, accessory molecule-1 (DNAM-1), one or more NCRs (e.g., NKp30, NKp44, NKp46), SLAM, Coxsackie virus and adenovirus receptor or a combination thereof.

Engineered γδ T cells

Engineered γδ T-cells may be generated with various methods known in the art. An engineered γδ T-cell may be designed to stably express a particular tumor recognition moiety. A polynucleotide encoding an expression cassette that comprises a tumor recognition, or another type of recognition moiety, can be stably introduced into the γδ T-cell by a transposon/transposase system or a viral-based gene transfer system, such as a lentiviral or a retroviral system, or another suitable method, such as transfection, electroporation, transduction, lipofection, calcium phosphate (CaPO₄), nanoengineered substances, such as Ormosil, viral delivery methods, including adenoviruses, retroviruses, lentiviruses, adeno-associated viruses, or another suitable method. An antigen specific TCR, either αβ or γδ, can be introduced into the engineered γδ T-cell by stably inserting a polynucleotide comprising a genetic code for the antigen specific TCR into the genome of the γδ T-cell. A polynucleotide encoding a CAR with a tumor recognition moiety may be introduced into the engineered γδ T-cell by stably inserting the polynucleotide into the genome of the γδ T-cell. In some cases, the engineered tumor recognition moiety is an engineered T-cell receptor, and the expression cassette incorporated into the genome of an engineered γδ T-cell comprises a polynucleotide encoding an engineered TCR a (TCR alpha) gene, an engineered TCR α (TCR beta) gene, an TCR β (TCR delta) gene, or an engineered TCR γ (TCR gamma) gene. In some cases, the expression cassette incorporated into the genome of the engineered γδ T-cell comprises a polynucleotide encoding an antibody fragment or an antigen binding portion thereof. In some cases, the antibody fragment or antigen binding fragment thereof is a polynucleotide encoding a whole antibody, an antibody fragment, a single-chain variable fragment (scFv), a single domain antibody (sdAb), a Fab, F(ab)₂, an Fc, the light or heavy chains on an antibody, the variable or the constant region of an antibody, or any combination thereof that binds to a cell surface tumor antigen as part of the Chimeric Antigen Receptor (CAR) construct, or a bi-specific construct, comprising a CAR and a T-cell receptor (TCR), or CARs with antibodies directed to different antigens. In some cases, the polynucleotide is derived from a human or from another species. An antibody fragment or antigen binding fragment polynucleotide that is derived from a non-human species can be modified to increase their similarity to antibody variants produced naturally in humans, and an antibody fragment or antigen binding fragment can be partially or fully humanized. An antibody fragment or antigen binding fragment polynucleotide can also be chimeric, for example a mouse-human antibody chimera. An engineered γδ T-cell that expresses a CAR can also be engineered to express a ligand to the antigen recognized by the tumor recognition moiety.

Various techniques known in the art can be used to introduce a cloned, or synthetically engineered, nucleic acid comprising the genetic code for a tumor recognition moiety into a specific location within the genome of an engineered γδ T-cell. The RNA-guided Cas9 nuclease from the microbial clustered regularly interspaced short palindromic repeats (CRISPR) system, zinc finger nucleases (ZFNs), transcription activator-like effector nucleases (TALENs), and meganuclease technologies, as described, respectively by WO201409370, WO2003087341, WO2014134412, and WO2011090804, each of which is incorporated by reference herein in its entireties, can be used to provide efficient genome engineering in γδ T-cell(s). The technologies described herein can also be used to insert the expression cassette into a genomic location that simultaneously provides a knock-out of one gene and a knock-in of another gene. For example, a polynucleotide comprising an expression cassette of the disclosure can be inserted into a genomic region that encodes for an WIC gene. Such engineering can simultaneously provide the knock-in of one or more genes, e.g. the genes comprised in the expression cassette, and a knock-out of another gene, e.g. an WIC locus.

In one case, a Sleeping Beauty transposon that includes a nucleic acid coding for the tumor recognition moiety is introduced into the cell γδ T-cell that is being engineered. A mutant Sleeping Beauty transposase that provides for enhanced integration as compared to the wild-type Sleeping Beauty, such as the transposase described in U.S. Pat. No. 7,985,739, which is incorporated by reference herein in its entirety, may be used to introduce a polynucleotide in the engineered γδ T-cell.

In some cases, a viral method is used to introduce a polynucleotide comprising a tumor recognition moiety into the genome of an engineered γδ T-cell. A number of viral methods have been used for human gene therapy, such as the methods described in WO 1993020221, which is incorporated herein in its entirety. Non-limiting examples of viral methods that can be used to engineer a γδ T-cell include retroviral, adenoviral, lentiviral, herpes simplex virus, vaccinia virus, pox virus, or adeno-virus associated viral methods.

A polynucleotide containing the genetic code for a tumor recognition moiety may comprise mutations or other transgenes that affect the growth, proliferation, activation status of the engineered γδ T-cell or an antigen specific to tumor cells such as testis-specific cancer antigens. A γδ T-cell of the disclosure may be engineered to express a polynucleotide comprising an activation domain that is linked to the antigen recognition moiety, such as a molecule in TCR-CD3 complex or a co-stimulatory factor. An engineered γδ T-cell can express an intracellular signaling domain that is a T-lymphocyte activation domain. The γδ T-cell may be engineered to express an intracellular activation domain gene or an intracellular signaling domain. The intracellular signaling domain gene, may be, for example CD3, CD28, CD2, ICOS, JAML, CD27, CD30, OX40, NKG2D, CD4, OX40/CD134, 4-1BB/CD137, FcεRIγ, IL-2RB/CD 122, IL-2RG/CD132, DAP molecules, CD70, cytokine receptor, CD40, or any combination thereof. In some cases, the engineered γδ T-cell is also engineered to express a cytokine, an antigen, a cellular receptor, or other immunomodulatory molecule.

The appropriate tumor recognition moiety to be expressed by the engineered γδ T-cell can be selected based on the disease to be treated. For example, in some cases a tumor recognition moiety is a TCR. In some cases, a tumor recognition moiety is a receptor to a ligand that is expressed on a cancer cell. Non-limiting examples of suitable receptors include NKG2D, NKG2A, NKG2C, NKG2F, LLT1, AICL, CD26, NKRP1, CD244 (2B4), DNAM-1, NKp30, NKp44, NKp46, and NKp80. In some cases, a tumor recognition moiety can include a ligand, e.g. IL-13 ligand, or a ligand mimetic to the tumor antigen, such as the IL-13 mimetic to IL13R.

A γδ T-cell may be engineered to express a chimeric tumor recognition moiety comprising a ligand binding domain derived from NKG2D, NKG2A, NKG2C, NKG2F, LLT1, AICL, CD26, NKRP1, CD244 (2B4), DNAM-1, or an anti-tumor antibody such as anti-Her2neu or anti-EGFR and a signaling domain obtained from CD3-c Dap 10, Dap 12, CD28, 41BB, and CD40L. In some examples, the chimeric receptor binds MICA, MICB, Her2neu, EGFR, EGFRvIII, mesothelin, CD38, CD20, CD19, BCMA, PSA, RON, CD30, CD22, CD37, CD38, CD56, CD33, CD138, CD123, CD79b, CD70, CD75, CA6, GD2, alphafetoprotein (AFP), CS1, carcinoembryonic antigen (CEA), CEACAM5, CA-125, MUC-16, 5T4, NaPi2b, ROR1, ROR2, PLIF, Her2/Neu, EGFRvIII, GPMNB, LIV-1, glycolipidF77, fibroblast activation protein (FAP), PSMA, STEAP-1, STEAP-2, c-Met, CSPG4, CD44v6, PVRL-4, VEGFR2, C4.4a, PSCA, folate binding protein/receptor, SLC44A4, Cripto, CTAG1B, AXL, IL-13Ra2, IL-3R, EPHA3, SLTRK6, gp100, MART1, Tyrosinase, SSX2, SSX4, NYESO-1, epithelial tumor antigen (ETA), MAGEA family genes (such as MAGEA3. MAGEA4), KKLC1, mutated ras (H, N, K), BRaf, p53, β-catenin, EGFRT790, MHC class I chain-related molecule A (MICA), or MHC class I chain-related molecule B (MICB), or one or more antigens of HPV, CMV, or EBV.

In some cases, the tumor recognition moiety targets an MHC class I molecule (HLA-A, HLA-B, or HLA-C) in complex with a tumor-associated peptide. Methods and compositions for generating and using tumor recognition moieties that target a tumor-associated peptide in complex with a MEW class I molecule include those described in Weidanz et al., Int. Rev. Immunol. 30:328-40, 2011; Scheinberg et al, Oncotarget. 4(5):647-8, 2013; Cheever et al, Clin. Cancer Res. 15(17):5323-37, 2009; Dohan & Reiter Expert Rev Mol Med. 14:e6, 2012; Dao et al., Sci Transl Med. 2013 Mar. 13; 5(176):176ra33; U.S. Pat. No. 9,540,448; and WO 2017/011804. In some embodiments, the targeted tumor-associated peptide of the peptide MHC complex is a peptide of Wilms' tumor protein 1 (WT1), human telomerase reverse transcriptase (hTERT), survivin, mouse double minute 2 homolog (MDM2), cytochrome P450 (CYP1B), KRAS, or BRAF.

Two or more tumor recognition moieties may be expressed in the γδ T-cell from genetically different, substantially different, or substantially identical, αβ TCR polynucleotides stably expressed from the engineered γδ T-cell or from genetically distinct αβ TCR polynucleotides stably incorporated in the engineered γδ T-cell. In the case of genetically distinct αβ TCR(s), αβ TCR(s) recognizing different antigens associated with the same condition may be utilized. In one preferred embodiment, a γδ T-cell is engineered to express different TCRs, from human or mouse origin, from one or more expression cassettes that recognize the same antigen in the context of different MHC haplotypes. In another preferred embodiment, a γδ T-cell is engineered to express one TCR and two or more antibodies directed to the same or different peptides from a given antigen complexed with different MEW haplotypes. In some cases, expression of a single TCR by an engineered γδ T-cell facilitates proper TCR pairing. An engineered γδ T-cell that expresses different TCRs can provide a universal allogeneic engineered γδ T-cell. In a second preferred embodiment, a γδ T-cell is engineered to express one or more different antibodies directed to peptide-MHC complexes, each directed to the same or different peptide complexed with the same or different MHC haplotypes. In some cases, a tumor recognition moiety can be an antibody that binds to peptide-MHC complexes.

A γδ T-cell can be engineered to express TCRs from one or more expression cassettes that recognize the same antigen in the context of different MEW haplotypes. In some cases, an engineered γδ T-cell is designed to express a single TCR, or a TCR in combination with a CAR to minimize the likelihood of TCR mispairing within the engineered cell. The tumor recognition moieties expressed from two or more expression cassettes preferably have different polynucleotide sequences, and encode tumor recognition moieties that recognize different epitopes of the same target, e.g., in the context of different HLA haplotypes. An engineered γδ T-cell that expresses such different TCRs or CARs can provide a universal allogeneic engineered γδ T-cell.

In some cases, a γδ T-cell is engineered to express one or more tumor recognition moieties. Two or more tumor recognition moieties may be expressed from genetically identical, or substantially identical, antigen-specific chimeric (CAR) polynucleotides engineered in the γδ T-cell. Two or more tumor recognition moieties may be expressed from genetically distinct CAR polynucleotides engineered in the γδ T-cell. The genetically distinct CAR(s) may be designed to recognize different antigens associated with the same condition.

A γδ T-cell may alternatively be bi-specific. A bi-specific engineered γδ T-cell can express two or more tumor recognition moieties. A bi-specific engineered γδ T-cell can express both TCR and CAR tumor recognition moieties. A bi-specific engineered γδ T-cell can be designed to recognize different antigens associated with the same condition. An engineered γδ T-cell can express two or more CAR/TCR(s) bi-specific polynucleotides that recognize an identical or substantially identical antigen. An engineered γδ T-cell can express two or more CAR/TCR(s) bi-specific constructs that recognize distinct antigens. In some cases, a bi-specific construct of the disclosure binds to an activating and an inactivating domain of a target cell, thereby providing increased target specificity. The γδ T-cell may be engineered to express at least 1 tumor recognition moiety, at least 2 tumor recognition moieties, at least 3 tumor recognition moieties, at least 4 tumor recognition moieties, at least 5 tumor recognition moieties, at least 6 tumor recognition moieties, at least 7 tumor recognition moieties, at least 8 tumor recognition moieties, at least 9 tumor recognition moieties, at least 10 tumor recognition moieties, at least 11 tumor recognition moieties, at least 12 tumor recognition moieties, or another suitable number of tumor recognition moieties.

Proper TCR function may be enhanced by two functioning ζ (zeta) proteins comprising ITAM motifs. Proper TCR function may also be enhanced by expression of αβ or γδ activation domains, such as CD3, CD28, CD2, CTLA4, ICOS, JAML, PD-1, CD27, CD30, 41-BB, OX40, NKG2D, HVEM, CD46, CD4, FcεRIγ, IL-2RB/CD122, IL-2RG/CD132, DAP molecules, and CD70. The expressed polynucleotide may include the genetic code for a tumor recognition moiety, a linker moiety, and an activation domain. Translation of the polynucleotide by the engineered γδ T-cell may provide a tumor recognition moiety and an activation domain linked by a protein linker. Often, the linker comprises amino acids that do not obstruct the folding of the tumor recognition moiety and the activation domain. A linker molecule can be at least about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, or about 20 amino acids in length. In some cases, at least 50%, at least 70% or at least 90% of the amino acids in the linker are serine or glycine.

In some cases, an activation domain can comprise one or more mutations. Suitable mutations may be, for example, mutations that render an activation domain constitutively active. Altering the identity of one or more nucleic acids changes the amino acid sequence of the translated amino acid. A nucleic acid mutation can be made such that the encoded amino acid is modified to a polar, non-polar, basic or acidic amino acid. A nucleic acid mutation can be made such that the tumor recognition moiety is optimized to recognize an epitope from a tumor. The engineered tumor recognition moiety, an engineered activation domain, or another engineered component of a γδ T-cell may include more than 1 amino acid mutation, 2 amino acid mutations, 3 amino acid mutations, 4 amino acid mutations, 5 amino acid mutations, 6 amino acid mutations, 7 amino acid mutations, 8 amino acid mutations, 9 amino acid mutations, 10 amino acid mutations, 11 amino acid mutations, 12 amino acid mutations, 13 amino acid mutations, 14 amino acid mutations, 15 amino acid mutations, 16 amino acid mutations, 17 amino acid mutations, 18 amino acid mutations, 19 amino acid mutations, 20 amino acid mutations, 21 amino acid mutations, 22 amino acid mutations, 23 amino acid mutations, 24 amino acid mutations, 25 amino acid mutations, 26 amino acid mutations, 27 amino acid mutations, 28 amino acid mutations, 29 amino acid mutations, 30 amino acid mutations, 31 amino acid mutations, 32 amino acid mutations, 33 amino acid mutations, 34 amino acid mutations, 35 amino acid mutations, 36 amino acid mutations, 37 amino acid mutations, 38 amino acid mutations, 39 amino acid mutations, 40 amino acid mutations, 41 amino acid mutations, 42 amino acid mutations, 43 amino acid mutations, 44 amino acid mutations, 45 amino acid mutations, 46 amino acid mutations, 47 amino acid mutations, 48 amino acid mutations, 49 amino acid mutations, or 50 amino acid mutations.

In some cases, a γδ T-cell of the disclosure does not express one or more MHC molecules. Deletion of one or more MHC loci in an engineered γδ T-cell can decrease the likelihood that the engineered γδ T-cell will be recognized by the host immune system. The human Major Histocompatibility Complex (MHC) loci, known as the human leukocyte antigen (HLA) system, comprises a large gene family that is expressed in antigen presenting cells, including γδ T-cells. The HLA-A, HLA-B, and HLA-C molecules function to present intracellular peptides as antigens to antigen presenting cells. The HLA-DP, HLA-DM, HLA-DOA, HLA-DOB, HLA-DQ, and HLA-DR molecules function to present extracellular peptides as antigens to antigen presenting cells. Some alleles of the HLA genes have been associated with GVHD, autoimmune disorders, and cancer. An engineered γδ T-cell described herein can be further engineered to lack, or to disrupt gene expression of one or more HLA genes. An engineered γδ T-cell described herein can be further engineered to lack, or to disrupt gene expression of one or more components of the MHC complex, such as complete deletion of one or more of the MHC genes, deletion of specific exons, or deletion of the β₂ microglobulin (B2m). Genetic excision or genetic disruption of at least one HLA gene can provides a clinically therapeutic γδ T-cell that can be administered to a subject with any HLA haplotype without causing host-versus-graft disease. An engineered γδ T-cell as described herein can be a universal donor for a human subject with any HLA haplotype.

A γδ T-cell can be engineered to lack one or various HLA locus (loci). An engineered γδ T-cell can be engineered to lack an HLA-A allele, an HLA-B allele, an HLA-C allele, an HLA-DR allele, an HLA-DQ allele, or an HLA-DP allele. In some cases, an HLA allele is associated with a human condition, such as an auto-immune condition. For instance, the HLA-B27 allele has been associated with arthritis and uveitis, the HLA-DR2 allele has been associated with systemic lupus erythematosus, and multiple sclerosis, the HLA-DR3 allele has been associated with 21-hydroxylase deficiency, the HLA-DR4 has been associated with rheumatoid arthritis and type 1 diabetes. An engineered γδ T-cell that lacks, for example, the HLA-B27 allele can be administered to a subject afflicted with arthritis without being readily recognized the immune system of the subject. In some cases, deletion of one or more HLA loci provides an engineered γδ T-cell that is a universal donor for any subject with any HLA haplotype.

In some cases, engineering a γδ T-cell requires the deletion of a portion of the γδ T-cell genome. In some cases, the deleted portion of the genome comprises a portion of the MHC locus (loci). In some instances, the engineered γδ T-cell is derived from a wild-type human γδ T-cell, and the MHC locus is an HLA locus. In some cases, the deleted a portion of the genome comprises a portion of a gene corresponding to a protein in the MHC complex. In some cases, the deleted portion of the genome comprises the β2 microglobulin gene. In some instances, the deleted portion of the genome comprises an immune checkpoint gene, such as PD-1, CTLA-4, LAG3, ICOS, BTLA, KIR, TIM3, A2aR, B7-H3, B7-H4, and CECAM-1. In some cases, an engineered γδ T-cell can be designed to express an activation domain that enhances T-cell activation and cytotoxicity. Non-limiting examples of activation domains that can be expressed by an engineered γδ T-cell include: CD2, ICOS, 4-1 BB (CD137), OX40 (CD134), CD27, CD70, CD80, CD86, DAP molecules, CD122, GITR, FcεRIγ.

Any portion of the genome of an engineered γδ T-cell can be deleted to disrupt the expression of an endogenous γδ T-cell gene. Non-limiting examples of genomic regions that can be deleted or disrupted in the genome of an γδ T-cell include a promoter, an activator, an enhancer, an exon, an intron, a non-coding RNA, a micro-RNA, a small-nuclear RNA, variable number tandem repeats (VNTRs), short tandem repeat (STRs), SNP patterns, hypervariable regions, minisatellites, dinucleotide repeats, trinucleotide repeats, tetranucleotide repeats, or simple sequence repeats. In some cases, the deleted a portion of the genome ranges between 1 nucleic acid to about 10 nucleic acids, 1 nucleic acid to about 100 nucleic acids, 1 nucleic acid to about 1,000 nucleic acids, 1 nucleic acid to about 10,000 nucleic acids, 1 nucleic acid to about 100,000 nucleic acids, 1 nucleic acid to about 1,000,000 nucleic acids, or other suitable range.

HLA gene expression in an engineered γδ T-cell can also be disrupted with various techniques known in the art. In some cases, large loci gene editing technologies are used to excise a gene from the engineered γδ T-cell genome, or to disrupt gene expression of at least one HLA locus in the engineered γδ T-cell. Non-limiting examples of gene editing technologies that can be used to edit a desired locus on a genome of an engineered γδ T-cell include Clustered Regularly Interspaced Short Palindromic Repeats (CRISPR)-Cas, zinc finger nucleases (ZFNs), Transcription activator-like effector nucleases (TALENs), and meganuclease technologies, as described, respectively by WO201409370, WO2003087341, WO2014134412, and WO 2011090804, and each of which is incorporated by reference herein in its entireties.

A γδ T-cell may be engineered from an isolated non-engineered γδ T-cell that already expresses a tumor recognition moiety. The engineered γδ T-cell can retain a tumor cell recognition moiety that is endogenously expressed by the isolated wild-type γδ T-cell, e.g., isolated from tumor infiltrating lymphocytes of a tumor sample. In some cases, the engineered γδ T-cell tumor cell recognition moiety replaces the wild-type γδ TCR.

A γδ T-cell can be engineered to express one or more homing molecules, such as a lymphocyte homing molecule. Homing molecules can be, for instance, lymphocyte homing receptors or cell adhesion molecules. A homing molecule can help an engineered γδ T-cell to migrate and infiltrate a solid tumor, including a targeted solid tumor upon administration of the engineered γδ T-cell to the subject. Non-limiting examples of homing receptors include members of the CCR family, e.g. CCR2, CCR4, CCR7, CCR8, CCR9, CCR10, CLA, CD44, CD103, CD62L, E-selectin, P-selectin, L-selectin, integrins, such as VLA-4 and LFA-1. Non-limiting examples of cell adhesion molecules include ICAM, N-CAM, VCAM, PE-CAM, L1-CAM, Nectins (PVRL1, PVRL2, PVRL3), LFA-1, integrin alphaXbeta2, alphavbeta7, macrophage-1 antigen, CLA-4, glycoprotein IIb/IIIa. Additional examples of cell adhesion molecules include calcium dependent molecules, such as T-cadherin, and antibodies to matrix metaloproteinases (MMPs) such as MMP9 or MMP2.

The steps involved in T-cell maturation, activation, proliferation, and function may be regulated through co-stimulatory and inhibitory signals through immune checkpoint proteins. Immune checkpoints are co-stimulatory and inhibitory elements intrinsic to the immune system. Immune checkpoints aid in maintaining self-tolerance and modulating the duration and amplitude of physiological immune responses to prevent injury to tissues when the immune system responds to disease conditions, such as cell transformation or infection. The equilibrium between the co-stimulatory and inhibitory signals used to control the immune response from either γδ and αβ T-cells can be modulated by immune checkpoint proteins. Immune checkpoint proteins, such as PD1 and CTLA4 are present on the surface of T-cells and can be used to turn an immune response “on” or “off.” Tumors can dysregulate checkpoint protein function as an immune-resistance mechanism, particularly against T-cells that are specific for tumor antigens. An engineered γδ T-cell of the disclosure can be further engineered to lack one or more immune checkpoint locus (loci), such as PD-1, CTLA-4, LAG3, ICOS, BTLA, KIR, TIM3, A2aR, CEACAM1, B7-H3, and B7-H4. Alternatively, the expression of an endogenous immune check point gene in an engineered γδ T-cell of the disclosure can be disrupted with gene editing technologies.

Immunological checkpoints can be molecules that regulate inhibitory signaling pathways (exemplified by CTLA4, PD1, and LAG3) or molecules that regulate stimulatory signaling pathways (exemplified by ICOS) in an engineered γδ T-cell of the disclosure. Several proteins in the extended immunoglobulin superfamily can be ligands for immunological checkpoints. Non-limiting examples of immune checkpoint ligand proteins include B7-H4, ICOSL, PD-L1, PD-L2, MegaCD40L, MegaOX40L, and CD137L. In some cases, immune checkpoint ligand proteins are antigens expressed by a tumor. In some cases, the immune checkpoint gene is a CTLA-4 gene. In some cases, the immune checkpoint gene is a PD-1 gene.

PD1 is an inhibitory receptor belonging to the CD28/CTLA4 family and is expressed on activated T lymphocytes, B cells, monocytes, DCs, and T-regs. There are two known ligands for PD1, PD-L1 and PD-L2, which are expressed on T cells, APCs, and malignant cells function to suppress self-reactive lymphocytes and to inhibit the effector function of TAA-specific cytotoxic T lymphocytes (CTLs). Accordingly, an engineered γδ T-cell that lacks PD1 can retain its cytotoxic activity regardless of expression of PD-L1 and PD-L2 by tumor cells. In some cases, an engineered γδ T-cell of the disclosure lacks the gene locus for the PD-1 gene. In some cases, expression of the PD-1 gene in an engineered γδ T-cell is disrupted by gene editing technologies.

CTLA4 (cytotoxic T-lymphocyte antigen 4) is also known as CD152 (Cluster of differentiation 152). CTLA4 shares sequence homology and ligands (CD80/B7-1 and CD86/B7-2) with the costimulatory molecule CD28, but differs by delivering inhibitory signals to T-cells expressing CTLA4 as a receptor. CTLA4 has a much higher overall affinity for both ligands and can out-compete CD28 for binding when ligand densities are limiting. CTLA4 is often expressed on the surface of CD8⁺ effector T-cells, and plays a functional role in the initial activation stages of both naive and memory T-cells. CTLA4 counteracts the activity of CD28 via increased affinity for CD80 and CD86 during the early stages of T-cell activation. The major functions of CTLA4 include down-modulation of helper T-cells and enhancement of regulatory T-cell immunosuppressive activity. In some instances, an engineered γδ T-cell of the disclosure lacks the CTLA4 gene. In some cases, expression of the CTLA4 gene in an engineered γδ T-cell is disrupted by gene editing technologies.

LAG3 (Lymphocyte-activation gene 3) is expressed on activated antigen-specific cytotoxic T-cells, and can enhance the function of regulatory T-cells and independently inhibit CD8⁺ effector T-cell activity. LAG3 is a CD-4-like negative regulatory protein with a high affinity binding to MHC Class II proteins, which are upregulated on some epithelial cancers, leading to tolerance of T cell proliferation and homeostasis. Reduction of the LAG-3/Class II interaction using a LAG-3-IG fusion protein may enhance antitumor immune responses. In some cases, an engineered γδ T-cell of the disclosure lacks the gene locus for the LAG3gene. In some instances, expression of the LAG3gene in an engineered γδ T-cell is disrupted by gene editing technologies.

Phenotype of Non-Engineered and Engineered γδ T-Cells

An engineered γδ T-cell may home to a specific physical location in a subject's body. Migration and homing of engineered γδ T cells, can be dependent on the combined expression and actions of specific chemokines and/or adhesion molecules. Homing of engineered γδ T cells can be controlled by the interactions between chemokines and their receptors. For example, cytokines including but not limited to CXCR3 (whose ligands are represented by IP-10/CXCL10 and 6Ckine/SLC/CCL21) CCR4+ CXCR5+ (receptor for RANTES, MIP-1α, MIP-1β), CCR6+ and CCR7 may affect homing of engineered γδ T cells. In some cases, an engineered γδ T-cell may home to sites of inflammation and injury, and to diseased cells to perform repair functions. In some cases, an engineered γδ T-cell can home to a cancer. In some cases, an engineered γδ T-cell may home to a thymus, a bone marrow, a skin, a larynx, a trachea, pleurae, a lung, an esophagus, an abdomen, a stomach, a small intestine, a large intestine, a liver, a pancreas, a kidney, a urethra, a bladder, a testis, a prostate, a ductus deferens, am ovary, an uretus, a mamary gland, a parathyroid gland, a spleen or another site in a subject's body. An engineered γδ T-cell can express one or more homing moieties, such as particular TCR allele and/or a lymphocyte homing molecule.

An engineered γδ T-cell may have a particular phenotype and a phenotype can be described in terms of cell-surface marker expression. Various types of γδ T-cells can be engineered as described herein. In preferred embodiments, the engineered γδ T-cell is derived from a human, but the engineered γδ T-cell may also be derived from a different source, such as a mammal or a synthetic cell.

The immunophenotype of the activated and/or expanded cell populations may be determined using markers including but not limited to CD137, CD27, CD45RA, CD45RO, CCR7 and CD62L (Klebanoff et al., Immunol Rev. 211: 214 2006). CD137, or 4-1BB, is an activation-induced costimulatory molecule and an important regulator of immune responses. Pollok et al., J. Immunol. 150, 771-81 (1993). CD45RA is expressed on naïve T lymphocytes, replaced by CD45RO upon antigen encounter, but re-expressed in late effector cells (Michie et al., Nature 360, 264-265 (1992); CD62L is a cell adhesion molecule that acts as a homing molecule to enter secondary lymphoid tissues and is lost after T-cell activation, when T-cells acquire effector functions (Sallusto et al., Nature. 401:708 (1999). CD27 is costimulation markers that are lost during T-cell differentiations (Appay et al., Nat Med. 8:379 (2002); Klebanoff et al., Immunol Rev. 211: 214 2006). Additional or alternative activation markers include, but are not limited to, one or more of CD25, PD-1, and CD69.

Antigens

The invention disclosed herein provides an engineered γδ T-cell that expresses an antigen recognition moiety, wherein the antigen recognition moiety recognizes a disease-specific epitope. An antigen may be a molecule that provokes an immune response. This immune response may involve either antibody production, the activation of specific immunologically-competent cells, or both. An antigen may be, for example, a peptide, a protein, a hapten, a lipid, a carbohydrate, bacteria, a pathogen, or a virus. An antigen may be a tumor antigen. A tumor epitope may be presented by the MHC I or MHC II complexes on the surface of tumor cells. An epitope can be the portion of the antigen that is expressed on the cell surface and recognized by the tumor recognition moiety.

Non-limiting examples of antigens recognized by an engineered γδ T-cell include CD19, CD20, CD30, CD22, CD37, CD38, CD56, CD33, CD138, CD123, CD79b, CD70, CD75, CA6, GD2, alphafetoprotein (AFP), carcinoembryonic antigen (CEA), RON, CEACAM5, CA-125, MUC-16, 5T4, NaPi2b, ROR1, ROR2, PLIF, Her2/Neu, EGFRvIII, GPMNB, LIV-1, glycolipidF77, fibroblast activation protein (FAP), PSMA, STEAP-1, STEAP-2, mesothelin, c-Met, CSPG4, PVRL-4, VEGFR2, PSCA, CLEC12a, L1CAM, GPC2, GPC3, folate binding protein/receptor, SLC44A4, Cripto, CTAG1B, AXL, IL-13R, IL-3Rα2, SLTRK6, gp100, MART1, Tyrosinase, SSX2, SSX4, NYESO-1, WT-1, PRAME, epithelial tumor antigen (ETA), MAGEA family genes (such as MAGEA3. MAGEA4), KKLC1, mutated ras, VRaf, p53, MHC class I chain-related molecule A (MICA), or MHC class I chain-related molecule B (MICB), or one or more antigens of HPV, CMV, or EBV.

An antigen can be expressed in the intracellular or the extracellular compartment of a cell and an engineered γδ T-cell can recognize an intracellular or an extracellular tumor antigen. In some cases, an αβ TCR in the engineered γδ T-cell recognizes a peptide derived from either an intracellular or an extracellular tumor antigen. For example, an antigen may be a protein intracellularly or extracellularly produced by a cell infected with a virus, such as an HIV, an EBV, a CMV, or an HPV protein. An antigen may also be a protein intracellularly or extracellularly expressed by a cancerous cell.

An antigen recognition moiety may recognize an antigen from a cell in distress, such as a cancerous cell or a cell that has been infected with a virus. For instance, the human MHC class I chain-related genes (MICA and MICB) are located within the HLA class I region of chromosome 6. MICA and MICB proteins are considered to be markers of “stress” in the human epithelia, and act as ligands for cells expressing a common natural killer-cell receptor (NKG2D). As stress markers, MICA and MICB can be highly expressed from cancerous cells. An engineered γδ T-cell can recognize a MICA or a MICB tumor epitope.

A tumor recognition moiety may be engineered to recognize an antigen with certain avidity. For instance, a tumor recognition moiety encoded by a TCR or CAR construct may recognize an antigen with a dissociation constant of at least at least 10 fM, at least 100 fM, at least 1 picomolar (pM), at least 10 pM, at least 20 pM, at least 30 pM, at least 40 pM, at least 50 pM, at least 60 pM, at least 7 pM, at least 80 pM, at least 90 pM, at least 100 pM, at least 200 pM, at least 300 pM, at least 400 pM, at least 500 pM, at least 600 pM, at least 700 pM, at least 800 pM, at least 900 pM, at least 1 nanomolar (nM), at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nm, at least 60 nM, at least 70 nM, at least 80 nM, at least 90 nM, at least 100 nM, at least 200 nM, at least 300 nM, at least 400 nM, at least 500 nM, at least 600 nM, at least 700 nM, at least 800 nM, at least 900 nM, at least 1 at least 2 at least 3 at least 4 at least 5 at least 6 at least 7 at least 8 at least 9 at least 10 at least 20 at least 30 at least 40 at least 50 at least 60 at least 70 at least 80 at least 90 or at least 100 μM.

In some instances, a tumor recognition moiety may be engineered to recognize an antigen with a dissociation constant of at most 10 fM, at most 100 fM, at most 1 picomolar (pM), at most 10 pM, at most 20 pM, at most 30 pM, at most 40 pM, at most 50 pM, at most 60 pM, at most 7 pM, at most 80 pM, at most 90 pM, at most 100 pM, at most 200 pM, at most 300 pM, at most 400 pM, at most 500 pM, at most 600 pM, at most 700 pM, at most 800 pM, at most 900 pM, at most 1 nanomolar (nM), at most 2 nM, at most 3 nM, at most 4 nM, at most 5 nM, at most 6 nM, at most 7 nM, at most 8 nM, at most 9 nM, at most 10 nM, at most 20 nM, at most 30 nM, at most 40 nM, at most 50 nm, at most 60 nM, at most 70 nM, at most 80 nM, at most 90 nM, at most 100 nM, at most 200 nM, at most 300 nM, at most 400 nM, at most 500 nM, at most 600 nM, at most 700 nM, at most 800 nM, at most 900 nM, at most 1 μM, at most 2 μM, at most 3 μM, at most 4 μM, at most 5 μM, at most 6 μM, at most 7 μM, at most 8 μM, at most 9 μM, at most 10 μM, at most 20 μM, at most 30 μM, at most 40 μM, at most 50 μM, at most 60 μM, at most 70 μM, at most 80 μM, at most 90 μM, or at most 100 μM.

Methods of Treatment

Pharmaceutical compositions containing a non-engineered, enriched γδ T-cell population, an engineered, enriched γδ T-cell population, and/or admixtures thereof, as described herein may be administered for prophylactic and/or therapeutic treatments. Additionally or alternatively, pharmaceutical compositions containing one or more agents that selectively expand a γδ T-cell population, as described herein, may be administered for prophylactic and/or therapeutic treatments. In therapeutic applications, the compositions can be administered to a subject already suffering from a disease or condition in an amount sufficient to cure or at least partially arrest the symptoms of the disease or condition. The compositions, can also be administered to lessen a likelihood of developing, contracting, or worsening a condition. Effective amounts of a population of a non-engineered, enriched γδ T-cell population, an engineered, enriched γδ T-cell population, admixtures thereof, and/or one or more agents that selectively expand a γδ T-cell population for therapeutic use can vary based on the severity and course of the disease or condition, previous therapy, the subject's health status, weight, and/or response to the drugs, and/or the judgment of the treating physician.

The compositions of the disclosure can be used to treat a subject in need of treatment for a condition. Examples of conditions include cancer, infectious disease, autoimmune disorder and sepsis. Subjects can be humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants.

Compositions, and combinations thereof, of the disclosure may be administered at various regimens (e.g., timing, concentration, dosage, spacing between treatment, and/or formulation). A subject can also be preconditioned with, for example, chemotherapy, radiation, or a combination of both, prior to receiving an in vivo expansion agent and/or an enriched γδ T-cell population, or admixtures thereof, of the disclosure. As part of a treatment, a composition may be administered to a subject at a first regimen and the subject may be monitored to determine whether the treatment at the first regimen meets a given level of therapeutic efficacy. In some cases, the engineered γδ T-cell or another engineered γδ T-cell may be administered to the subject at a second regimen.

In some embodiments, in a first operation, at least one composition described herein is administered to a subject that has or is suspected of having a given condition (e.g., cancer). The composition may be administered at a first regimen. In a second operation, the subject may be monitored, for example by a healthcare provider (e.g., treating physician or nurse). In some examples, the subject is monitored to determine or gauge an efficacy of the composition in treating the condition of the subject. In some situations, the subject may also be monitored to determine the in vivo activation, expansion, or cell number of a γδ T-cell population in the subject. Next, in a third operation, at least one composition described herein is administered to the subject at a second regimen. The second regimen may be the same as the first regimen or different than the first regimen. In some situations, the third operation is not performed, for example, if the administration of the composition in the first operation is found to be effective (e.g., a single round of administration may be sufficient to treat the condition). Due to their allogeneic and universal donor characteristics, a population of engineered γδ T-cells may be administered to various subjects, with different WIC haplotypes. An engineered γδ T-cell may be frozen or cryopreserved prior to being administered to a subject.

A enriched population of γδ T-cells (i.e., engineered or non-engineered) and/or admixtures thereof, may also be frozen or cryopreserved prior to being administered to a subject and optionally further activated and expanded and/or maintained in vivo by administration of one or more agents that selectively expand the administered γδ T-cells. In certain embodiments, a population of engineered, enriched γδ T-cells can comprise two or more cells that express identical, different, or a combination of identical and different tumor recognition moieties.

For instance, a population of engineered, enriched γδ T-cells can comprises several distinct engineered γδ T-cells that are designed to recognize different antigens, or different epitopes of the same antigen. For example, human cells afflicted with melanoma can express the NY-ESO-1 oncogene. Infected cells within the human can process the NY-ESO-1 oncoprotein into smaller fragments and present various portions of the NY-ESO-1 protein for antigen recognition. A population of engineered, enriched γδ T-cells can comprise various engineered γδ T-cells that express different tumor recognition moieties designed to recognize different portions of the NY-ESO-1 protein.

In some embodiments, the present invention provides a method for treating a subject with a population of engineered γδ T-cells that recognizes different epitopes of the melanoma antigen NY-ESO-1. In a first operation, a population of engineered γδ T-cells that recognize different epitopes of the same antigen is selected. For example, the population of engineered γδ T-cells may comprise two or more cells that expressing different tumor recognition moieties that recognize different portions of the NY-ESO-1 protein. In a second operation, The population of engineered γδ T-cells may be administered at a first regimen. In a second operation, the subject may be monitored, for example by a healthcare provider (e.g., treating physician or nurse). In a third operation, the subject may be administered one or more agents that selectively expand the administered γδ T-cells in vivo to thereby expand and/or maintain the administered population of γδ T-cells in vivo. In a fourth operation, the subject may be monitored to determine the efficacy of the in vivo expansion and/or maintenance. In some embodiments, the second operation is not performed. In some embodiments, the fourth operation is not performed.

One or more compositions of the disclosure may be used to treat various conditions. In some cases, a composition of the disclosure may be used to treat a cancer, including solid tumors and hematologic malignancies. Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

In some cases, a composition of the disclosure may be used to treat an infectious disease. An infectious disease may be caused, for example, by a pathogenic bacterium or by a virus. Various pathogenic proteins, nucleic acids, lipids, or fragments thereof can be expressed by a diseased cell. An antigen presenting cell can internalize such pathogenic molecules, for instance with phagocytosis or by receptor-mediated endocytosis, and display a fragment of the antigen bound to an appropriate MHC molecule. For instance, various 9 mer fragments of a pathogenic protein may be displayed by an APC. Engineered, enriched γδ T-cell populations of the disclosure may be designed to recognize various antigens and antigen fragments of a pathogenic bacterium or a virus. Non-limiting examples of pathogenic bacteria can be found in the: a) Bordetella genus, such as Bordetella pertussis species; b) Borrelia genus, such Borrelia burgdorferi species; c) Brucelia genus, such as Brucella abortus, Brucella canis, Brucela meliterisis, and/or Brucella suis species; d) Campylobacter genus, such as Campylobacter jejuni species; e) Chlamydia and Chlamydophila genuses, such as Chlamydia pneumonia, Chlamydia trachomatis, and/or Chlamydophila psittaci species; f) Clostridium genus, such as Clostridium botulinum, Clostridium difficile, Clostridium perfringens, Clostridium tetani species; g) Corynebacterium genus, such as Corynebacterium diphtheria species; h) Enterococcus genus, such as Enterococcus faecalis, and/or Enterococcus faecium species; i) Escherichia genus, such as Escherichia coli species; j) Francisella genus, such as Francisella tularensis species; k) Haemophilus genus, such as Haemophilus influenza species; 1) Helicobacter genus, such as Helicobacter pylori species; m) Legionella genus, such as Legionella pneumophila species; n) Leptospira genus, such as Leptospira interrogans species; o) Listeria genus, such as Listeria monocytogenes species; p) Mycobacterium genus, such as Mycobacterium leprae, Mycobacterium tuberculosis, and/or mycobacterium ulcerans species; q) Mycoplasma genus, such as Mycoplasma pneumonia species; r) Neisseria genus, such as Neisseria gonorrhoeae and/or Neisseria meningitidia species; s) Pseudomonas genus, such as Pseudomonas aeruginosa species; t) Rickettsia genus, such as Rickettsia rickettsii species; u) Salmonella genus, such as Salmonella typhi and/or Salmonella typhimurium species; v) Shigella genus, such as Shigella sonnei species; w) Staphylococcus genus, such as Staphylococcus aureus, Staphylococcus epidermidis, and/or Staphylococcus saprophyticus species; x) Streptococcus genus, such as Streptococcus agalactiae, Streptococcus pneumonia, and/or Streptococcus pyogenes species; y) Treponema genus, such as Treponema pallidum species; z) Vibrio genus, such as Vibrio cholera; and/or aa) Yersinia genus, such as Yersinia pestis species.

In some cases, a composition of the disclosure may be used to treat an infectious disease, an infectious disease may be caused a virus. Non-limiting examples of viruses can be found in the following families of viruses and are illustrated with exemplary species: a) Adenoviridae family, such as Adenovirus species; b) Herpesviridae family, such as Herpes simplex type 1, Herpes simplex type 2, Varicella-zoster virus, Epstein-barr virus, Human cytomegalovirus, Human herpesvirus type 8 species; c) Papillomaviridae family, such as Human papillomavirus species; d) Polyomaviridae family, such as BK virus, JC virus species; e) Poxviridae family, such as Smallpox species; f) Hepadnaviridae family, such as Hepatitis B virus species; g) Parvoviridae family, such as Human bocavirus, Parvovirus B19 species; h) Astroviridae family, such as Human astrovirus species; i) Caliciviridae family, such as Norwalk virus species; j) Flaviviridae family, such as Hepatitis C virus (HCV), yellow fever virus, dengue virus, West Nile virus species; k) Togaviridae family, such as Rubella virus species; 1) Hepeviridae family, such as Hepatitis E virus species; m) Retroviridae family, such as Human immunodeficiency virus (HIV) species; n) Orthomyxoviridaw family, such as Influenza virus species; o) Arenaviridae family, such as Guanarito virus, Junin virus, Lassa virus, Machupo virus, and/or Sabia virus species; p) Bunyaviridae family, such as Crimean-Congo hemorrhagic fever virus species; q) Filoviridae family, such as Ebola virus and/or Marburg virus species; Paramyxoviridae family, such as Measles virus, Mumps virus, Parainfluenza virus, Respiratory syncytial virus, Human metapneumovirus, Hendra virus and/or Nipah virus species; r) Rhabdoviridae genus, such as Rabies virus species; s) Reoviridae family, such as Rotavirus, Orbivirus, Coltivirus and/or Banna virus species. In some examples, a virus is unassigned to a viral family, such as Hepatitis D.

In some cases, a composition of the disclosure may be used to treat an immune disease, such as an autoimmune disease. Inflammatory diseases, including autoimmune diseases are also a class of diseases associated with B-cell disorders. Examples of immune diseases or conditions, including autoimmune conditions, include: rheumatoid arthritis, rheumatic fever, multiple sclerosis, experimental autoimmune encephalomyelitis, psoriasis, uveitis, diabetes mellitus, systemic lupus erythematosus (SLE), lupus nephritis, eczema, scleroderma, polymyositis/scleroderma, polymyositis/dermatomyositis, ulcerative proctitis, ulcerative colitis, severe combined immunodeficiency (SCID), DiGeorge syndrome, ataxia-telangiectasia, seasonal allergies, perennial allergies, food allergies, anaphylaxis, mastocytosis, allergic rhinitis, atopic dermatitis, Parkinson's, Alzheimer's, hypersplenism, leukocyte adhesion deficiency, X-linked lymphoproliferative disease, X-linked agammaglobulinemia, selective immunoglobulin A deficiency, hyper IgM syndrome, HIV, autoimmune lymphoproliferative syndrome, Wiskott-Aldrich syndrome, chronic granulomatous disease, common variable immunodeficiency (CVID), hyperimmunoglobulin E syndrome, Hashimoto's thyroiditis, acute idiopathic thrombocytopenic purpura, chronic idiopathic thrombocytopenia purpura, dermatomyositis, Sydenham' a chorea, myasthenia gravis, polyglandular syndromes, bullous pemphigoid, Henoch-Schonlein purpura, poststreptococcalnephritis, erythema nodosum, erythema multiforme, gA nephropathy, Takayasu's arteritis, Addison's disease, sarcoidosis, ulcerative colitis, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, chronic active hepatitis, polychondritis, pamphigus vulgaris, Wegener's granulomatosis, membranous nephropathy, amyotrophic lateral sclerosis, tabes dorsalis, giant cell arteritis, /polymyalgia, peraiciousanemia, rapidly progressive glomerulonephritis, psoriasis, fibrosing alveolitis, and cancer.

Treatment with a composition of the disclosure may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition comprising one or more agents that selectively expand a γδ T-cell population. A treatment can comprise administering to a subject a pharmaceutical composition comprising a non-engineered, enriched γδ T-cell population, an engineered, enriched γδ T-cell population, and/or admixture thereof, of the disclosure. In some cases, the pharmaceutical composition comprises one or more agents of the disclosure that selectively expands a γδ T-cell population and a non-engineered, enriched γδ T-cell population, an engineered, enriched γδ T-cell population, and/or admixture thereof, of the disclosure.

In some cases, administration of a composition of the disclosure to a subject modulates the activity of endogenous lymphocytes in a subject's body. In some cases, administration of the composition of the disclosure to a subject provides an antigen to an endogenous T-cell and may boost an immune response. In some cases, the memory T-cell is a CD4⁺ T-cell. In some cases, the memory T-cell is a CD8⁺ T-cell. In some cases, administration of the composition of the disclosure to a subject activates the cytotoxicity of another immune cell. In some cases, the other immune cell is a CD8+ T-cell. In some cases, the other immune cell is a Natural Killer T-cell. In some cases, administration of the composition to a subject suppresses a regulatory T-cell. In some cases, the regulatory T-cell is a Fox3+ Treg cell. In some cases, the regulatory T-cell is a Fox3-Treg cell. Non-limiting examples of cells whose activity can be modulated by a γδ T-cell population include: hematopioietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells.

During most bone marrow transplants, a combination of cyclophosphamide with total body irradiation is conventionally employed to prevent rejection of the hematopietic stem cells (HSC) in the transplant by the subject's immune system. In some cases, incubation of donor bone marrow with interleukin-2 (IL-2) ex vivo is performed to enhance the generation of killer lymphocytes in the donor marrow. Interleukin-2 (IL-2) is a cytokine that is necessary for the growth, proliferation, and differentiation of wild-type lymphocytes. Current studies of the adoptive transfer of γδ T-cells into humans may require the co-administration of γδ T-cells and interleukin-2. However, both low- and high-dosages of IL-2 can have highly toxic side effects. IL-2 toxicity can manifest in multiple organs/systems, most significantly the heart, lungs, kidneys, and central nervous system. In some cases, the disclosure provides a method for administrating a non-engineered, enriched γδ T-cell population, an engineered, enriched γδ T-cell population, and/or admixtures thereof, to a subject without the co-administration of a cytokine, such as IL-2, IL-15, IL-12, or IL-21. In some cases, a non-engineered, enriched γδ T-cell population, an engineered, enriched γδ T-cell population, and/or admixtures thereof, can be administered to a subject without co-administration with IL-2. In some cases, a non-engineered, enriched γδ T-cell population, an engineered, enriched γδ T-cell population, and/or admixtures thereof, is administered to a subject during a procedure, such as a bone marrow transplant without the co-administration of IL-2.

In some cases, the disclosure provides a method for administrating a non-engineered, enriched γδ T-cell population, an engineered, enriched γδ T-cell population, and/or admixtures thereof, to a subject with the simultaneous or sequential co-administration of a cytokine or other stimulating agent such as IL-2, IL-4, IL-7, IL-9, IL-12, IL-15, IL-18, IL-19, IL-21, IL 23, IL-33, IFNγ, granulocyte-macrophage colony stimulating factor (GM-CSF), or granulocyte colony stimulating factor (G-CSF). In some cases, the cytokine is IL-2, IL-15, IL-12, or IL-21. In some cases, the cytokine is IL-2. In some cases, the cytokine is IL-15. In some cases, the cytokine is IL-4. In some cases, the cytokine is a common gamma chain cytokine selected from the group consisting of IL-2, IL-4, IL-7, IL-9, IL-15, and IL-21, or a combination thereof.

Methods of Administration

One or multiple compositions of the invention including a selective expansion agent; a non-engineered, enriched γδ T-cell population; an engineered, enriched γδ T-cell population; and/or admixtures thereof, can be administered to a subject in any order or simultaneously. If simultaneously, the compositions can be provided in a single, unified form, such as an intravenous injection, or in multiple forms, for example, as multiple intravenous infusions, s.c, injections or pills. The compositions can be packed together or separately, in a single package or in a plurality of packages. One or all of the compositions of the invention can be given in multiple doses. If not simultaneous, the timing between the multiple doses may vary to as much as about a week, a month, two months, three months, four months, five months, six months, or about a year. In some cases, an administered γδ T-cell population; engineered, enriched γδ T-cell population; and/or admixtures thereof, can expand within a subject's body, in vivo, after administration to a subject. Pharmaceutical compositions comprising γδ T-cell and/or activation agents can be packaged as a kit. A kit may include instructions (e.g., written instructions) on the use of the compositions, in addition to one or more of the compositions described herein.

In some cases, a method of treating a cancer comprises administering a composition described herein, wherein the administration treats the cancer. In some embodiments the therapeutically-effective amount of the composition, is administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year.

One or more compositions described herein can be administered before, during, or after the occurrence of a disease or condition, and the timing of administering a pharmaceutical composition can vary. For example, the one or more compositions can be used as a prophylactic and can be administered continuously to subjects with a propensity to conditions or diseases in order to lessen a likelihood of the occurrence of the disease or condition. The one or more compositions can be administered to a subject during or as soon as possible after the onset of the symptoms. The administration of the one or more compositions can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. In some examples, the administration of the one or more compositions of the disclosure is an intravenous administration. One or multiple dosages of one or more compositions can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of one or more compositions can be administered years after onset of the cancer and before or after other treatments. In some examples, one or more compositions described herein can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

Dosages

A non-engineered, enriched γδ T-cell population, an engineered, enriched γδ T-cell population, and/or admixtures thereof, as disclosed herein may be formulated in unit dosage forms suitable for single administration of precise dosages. In some cases, the unit dosage forms comprise additional lymphocytes. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative or without a preservative. In some examples, the pharmaceutical composition does not comprise a preservative. Formulations for parenteral injection can be presented in unit dosage form, for example, in ampoules, or in multi-dose containers with a preservative.

A non-engineered, enriched γδ T-cell population, an engineered, enriched γδ T-cell population, and/or admixtures thereof, as described herein may be present in a composition in an amount of at least 5 cells, at least 10 cells, at least 20 cells, at least 30 cells, at least 40 cells, at least 50 cells, at least 60 cells, at least 70 cells, at least 80 cells, at least 90 cells, at least 100 cells, at least 200 cells, at least 300 cells, at least 400 cells, at least 500 cells, at least 600 cells, at least 700 cells, at least 800 cells, at least 900 cells, at least 1×10³ cells, at least 2×10³ cells, at least 3×10³ cells, at least 4×10³ cells, at least 5×10³ cells, at least 6×10³ cells, at least 7×10³ cells, at least 8×10³ cells, at least 9×10³ cells, at least 1×10⁴ cells, at least 2×10⁴ cells, at least 3×10⁴ cells, at least 4×10⁴ cells, at least 5×10⁴ cells, at least 6×10⁴ cells, at least 7×10⁴ cells, at least 8×10⁴ cells, at least 9×10⁴ cells, at least 1×10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells, at least 2×10⁷ cells, at least 3×10⁷ cells, at least 4×10⁷ cells, at least 5×10⁷ cells, at least 6×10⁷ cells, at least 7×10⁷ cells, at least 8×10⁷ cells, at least 9×10⁷ cells, at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, or more.

The therapeutically effective dose of a non-engineered, enriched γδ T-cell population, an engineered, enriched γδ T-cell population, and/or admixtures thereof, of the invention can be from about 1 cell to about 10 cells, from about 1 cell to about 100 cells, from about 1 cell to about 10 cells, from about 1 cell to about 20 cells, from about 1 cell to about 30 cells, from about 1 cell to about 40 cells, from about 1 cell to about 50 cells, from about 1 cell to about 60 cells, from about 1 cell about 70 cells, from about 1 cell to about 80 cells, from about 1 cell to about 90 cells, from about 1 cell to about 100 cells, from about 1 cell to about 1×10³ cells, from about 1 cell to about 2×10³ cells, from about 1 cell to about 3×10³ cells, from about 1 cell to about 4×10³ cells, from about 1 cell to about 5×10³ cells, from about 1 cell to about 6×10³ cells, from about 1 cell to about 7×10³ cells, from about 1 cell to about 8×10³ cells, from about 1 cell to about 9×10³ cells, from about 1 cell to about 1×10⁴ cells, from about 1 cell to about 2×10⁴ cells, from about 1 cell to about 3×10⁴ cells, from about 1 cell to about 4×10⁴ cells, from about 1 cell to about 5×10⁴ cells, from about 1 cell to about 6×10⁴ cells, from about 1 cell to about 7×10⁴ cells, from about 1 cell to about 8×10⁴ cells, from about 1 cell to about 9×10⁴ cells, from about 1 cell to about 1×10⁵ cells, from about 1 cell to about 2×10⁵ cells, from about 1 cell to about 3×10⁵ cells, from about 1 cell to about 4×10⁵ cells, from about 1 cell to about 5×10⁵ cells, from about 1 cell to about 6×10⁵ cells, from about 1 cell to about 7×10⁵ cells, from about 1 cell to about 8×10⁵ cells, from about 1 cell to about 9×10⁵ cells, from about 1 cell to about 1×10⁶ cells, from about 1 cell to about 2×10⁶ cells, from about 1 cell to about 3×10⁶ cells, from about 1 cell to about 4×10⁶ cells, from about 1 cell to about 5×10⁶ cells, from about 1 cell to about 6×10⁶ cells, from about 1 cell to about 7×10⁶ cells, from about 1 cell to about 8×10⁶ cells, from about 1 cell to about 9×10⁶ cells, from about 1 cell to about 1×10⁷ cells, from about 1 cell to about 2×10⁷ cells, from about 1 cell to about 3×10⁷ cells, from about 1 cell to about 4×10⁷ cells, from about 1 cell to about 5×10⁷ cells, from about 1 cell to about 6×10⁷ cells, from about 1 cell to about 7×10⁷ cells, from about 1 cell to about 8×10⁷ cells, from about 1 cell to about 9×10⁷ cells, from about 1 cell to about 1×10⁸ cells, from about 1 cell to about 2×10⁸ cells, from about 1 cell to about 3×10⁸ cells, from about 1 cell to about 4×10⁸ cells, from about 1 cell to about 5×10⁸ cells, from about 1 cell to about 6×10⁸ cells, from about 1 cell to about 7×10⁸ cells, from about 1 cell to about 8×10⁸ cells, from about 1 cell to about 9×10⁸ cells, or from about 1 cell to about 1×10⁹ cells.

In some cases, the therapeutically effective dose of a non-engineered, enriched γδ T-cell population, an engineered, enriched γδ T-cell population, and/or admixtures thereof, of the invention can be from about 1×10³ cells to about 2×10³ cells, from about 1×10³ cells to about 3×10³ cells, from about 1×10³ cells to about 4×10³ cells, from about 1×10³ cells to about 5×10³ cells, from about 1×10³ cells to about 6×10³ cells, from about 1×10³ cells to about 7×10³ cells, from about 1×10³ cells to about 8×10³ cells, from about 1×10³ cells to about 9×10³ cells, from about 1×10³ cells to about 1×10⁴ cells, from about 1×10³ cells to about 2×10⁴ cells, from about 1×10³ cells to about 3×10⁴ cells, from about 1×10³ cells to about 4×10⁴ cells, from about 1×10³ cells to about 5×10⁴ cells, from about 1×10³ cells to about 6×10⁴ cells, from about 1×10³ cells to about 7×10⁴ cells, from about 1×10³ cells to about 8×10⁴ cells, from about 1×10³ cells to about 9×10⁴ cells, from about 1×10³ cells to about 1×10⁵ cells, from about 1×10³ cells to about 2×10⁵ cells, from about 1×10³ cells to about 3×10⁵ cells, from about 1×10³ cells to about 4×10⁵ cells, from about 1×10³ cells to about 5×10⁵ cells, from about 1×10³ cells to about 6×10⁵ cells, from about 1×10³ cells to about 7×10⁵ cells, from about 1×10³ cells to about 8×10⁵ cells, from about 1×10³ cells to about 9×10⁵ cells, from about 1×10³ cells to about 1×10⁶ cells, from about 1×10³ cells to about 2×10⁶ cells, from about 1×10³ cells to about 3×10⁶ cells, from about 1×10³ cells to about 4×10⁶ cells, from about 1×10³ cells to about 5×10⁶ cells, from about 1×10³ cells to about 6×10⁶ cells, from about 1×10³ cells to about 7×10⁶ cells, from about 1×10³ cells to about 8×10⁶ cells, from about 1×10³ cells to about 9×10⁶ cells, from about 1×10³ cells to about 1×10⁷ cells, from about 1×10³ cells to about 2×10⁷ cells, from about 1×10³ cells to about 3×10⁷ cells, from about 1×10³ cells to about 4×10⁷ cells, from about 1×10³ cells to about 5×10⁷ cells, from about 1×10³ cells to about 6×10⁷ cells, from about 1×10³ cells to about 7×10⁷ cells, from about 1×10³ cells to about 8×10⁷ cells, from about 1×10³ cells to about 9×10⁷ cells, from about 1×10³ cells to about 1×10⁸ cells, from about 1×10³ cells to about 2×10⁸ cells, from about 1×10³ cells to about 3×10⁸ cells, from about 1×10³ cells to about 4×10⁸ cells, from about 1×10³ cells to about 5×10⁸ cells, from about 1×10³ cells to about 6×10⁸ cells, from about 1×10³ cells to about 7×10⁸ cells, from about 1×10³ cells to about 8×10⁸ cells, from about 1×10³ cells to about 9×10⁸ cells, or from about 1×10³ cells to about 1×10⁹ cells.

In some cases, the therapeutically effective dose of a non-engineered, enriched γδ T-cell population, an engineered, enriched γδ T-cell population, and/or admixtures thereof, of the invention can be from about 1×10⁶ cells to about 2×10⁶ cells, from about 1×10⁶ cells to about 3×10⁶ cells, from about 1×10⁶ cells to about 4×10⁶ cells, from about 1×10⁶ cells to about 5×10⁶ cells, from about 1×10⁶ cells to about 6×10⁶ cells, from about 1×10⁶ cells to about 7×10⁶ cells, from about 1×10⁶ cells to about 8×10⁶ cells, from about 1×10⁶ cells to about 9×10⁶ cells, from about 1×10⁶ cells to about 1×10⁷ cells, from about 1×10⁶ cells to about 2×10⁷ cells, from about 1×10⁶ cells to about 3×10⁷ cells, from about 1×10⁶ cells to about 4×10⁷ cells, from about 1×10⁶ cells to about 5×10⁷ cells, from about 1×10⁶ cells to about 6×10⁷ cells, from about 1×10⁶ cells to about 7×10⁷ cells, from about 1×10⁶ cells to about 8×10⁷ cells, from about 1×10⁶ cells to about 9×10⁷ cells, from about 1×10⁶ cells to about 1×10⁸ cells, from about 1×10⁶ cells to about 2×10⁸ cells, from about 1×10⁶ cells to about 3×10⁸ cells, from about 1×10⁶ cells to about 4×10⁸ cells, from about 1×10⁶ cells to about 5×10⁸ cells, from about 1×10⁶ cells to about 6×10⁸ cells, from about 1×10⁶ cells to about 7×10⁸ cells, from about 1×10⁶ cells to about 8×10⁸ cells, from about 1×10⁶ cells to about 9×10⁸ cells, from about 1×10⁶ cells to about 1×10⁹ cells, from about 1×10⁶ cells to about 2×10⁹ cells, from about 1×10⁶ cells to about 3×10⁹ cells, from about 1×10⁶ cells to about 4×10⁹ cells, from about 1×10⁶ cells to about 5×10⁹ cells, from about 1×10⁶ cells to about 6×10⁹ cells, from about 1×10⁶ cells to about 7×10⁹ cells, from about 1×10⁶ cells to about 8×10⁹ cells, from about 1×10⁶ cells to about 9×10⁹ cells, from about 1×10⁷ cells to about 1×10⁹ cells, from about 1×10⁷ cells to about 2×10⁹ cells, from about 1×10⁷ cells to about 3×10⁹ cells, from about 1×10⁷ cells to about 4×10⁹ cells, from about 1×10⁷ cells to about 5×10⁹ cells, from about 1×10⁷ cells to about 6×10⁹ cells, from about 1×10⁷ cells to about 7×10⁹ cells, from about 1×10⁷ cells to about 8×10⁹ cells, from about 1×10⁷ cells to about 9×10⁹ cells, from about 1×10⁸ cells to about 1×10⁹ cells, from about 1×10⁸ cells to about 2×10⁹ cells, from about 1×10⁸ cells to about 3×10⁹ cells, from about 1×10⁸ cells to about 4×10⁹ cells, from about 1×10⁸ cells to about 5×10⁹ cells, from about 1×10⁸ cells to about 6×10⁹ cells, from about 1×10⁸ cells to about 7×10⁹ cells, from about 1×10⁸ cells to about 8×10⁹ cells, from about 1×10⁸ cells to about 9×10⁹ cells, or from about 1×10⁹ cells to about 1×10¹⁰ cells.

When an antibody or other activation agent is administered, such as an agent that binds the same or essentially the same epitope as, or competes with, an antibody described in any one of FIGS. 1-5, the normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

For the treatment or reduction in the severity of immune related disease, the appropriate dosage of a composition of the invention will depend on the type of disease to be treated, as defined above, the severity and course of the disease, whether the agent is administered for preventive or therapeutic purposes, previous therapy, a patient's clinical history and response to the compound, and the discretion of the attending physician. The composition can be suitably administered to the subject at one time or over a series of treatments.

For example, depending on the type and severity of the disease, about 1 mg/kg to 15 mg/kg (e.g., 0.1-20 mg/kg) of activation agent (e.g., polypeptide or antibody) is an initial candidate dosage for administration to the subject, whether, for example, by one or more separate administrations, or by continuous infusion. A typical daily dosage might range from about 1 mg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

Preservation

In some embodiments, enriched γδ T-cell populations, and/or admixtures thereof, obtained by ex vivo expansion of an in vivo activated or expanded γδ T-cell population may be formulated in freezing media and placed in cryogenic storage units such as liquid nitrogen freezers (−195° C.) or ultra-low temperature freezers (−65° C., −80° C. or −120° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved γδ T-cells can be thawed and further processed by stimulation with antibodies, proteins, peptides, and/or cytokines as described herein. The cryopreserved γδ T-cells can be thawed and genetically modified with viral vectors (including retroviral and lentiviral vectors) or non-viral means (including RNA, DNA, and proteins) as described herein. In some cases, non-engineered γδ T-cells can be expanded by the methods described herein, wherein the method includes a step of in vivo expansion, genetically modified, and cryopreserved.

Thus, genetically engineered and/or non-engineered γδ T-cells can be further cryopreserved to generate cell banks in quantities of at least about 1, 5, 10, 100, 150, 200, 500 vials at about at least 10¹, 10², 10³, 10⁴, 10⁵, 10⁶, 10⁷, 10⁸, 10⁹, or at least about 10¹⁰ cells per mL in freeze media. The cryopreserved cell banks may retain their functionality and can be thawed and further stimulated and expanded. In some aspects, thawed cells can be stimulated and expanded in suitable closed vessels such as cell culture bags and/or bioreactors to generate quantities of cells as allogeneic cell product. Cryopreserved γδ T-cells can maintain their biological functions for at least about 6 months, 7 months, 8 months, 9 months, 10 months, 11 months, 12 months, 13 months, 15 months, 18 months, 20 months, 24 months, 30 months, 36 months, 40 months, 50 months, or at least about 60 months under cryogenic storage condition. In some aspects, no preservatives are used in the formulation. The cryopreserved γδ T-cells can be thawed and administered to (e.g., infused into) multiple patients as allogeneic off-the-shelf cell product. The infused cells can be expanded and/or maintained in the administered subject(s) by administering one or more agents described herein that selectively expand γδ T-cells.

All publications and patents mentioned herein are incorporated herein by reference in their entirety for the purpose of describing and disclosing, for example, the constructs and methodologies that are described in the publications, which might be used in connection with the presently described inventions. The publications discussed herein are provided solely for their disclosure prior to the filing date of the present application. Nothing herein is to be construed as an admission that the inventors described herein are not entitled to antedate such disclosure by virtue of prior invention or for any other reason.

### EXAMPLES

### Example 1. Treatment of Tumors by In Vivo Expansion of γδ T Cells with One or More Agents that Selectively Bind δ1 TCR, Ω TCR, and/or δ3 TCR

The effect of administration of γδ T cell activating agents on the expansion and activation of human γδ T cells and treatment of tumors is tested in xenografts mouse models. Different hematological tumors cell lines (such as Raji, Daudi, Mino, NALM6, JVM-2, HL-60, MOLM-13, K562, KG-la, Mv4-11, MOLT-4 or others) and solid tumor cell lines (such as HCT116, colon cancer COL0205, melanoma SK-MELS, pancreatic BcPC3 or ASPC-1, breast cancer such as MDA-MB-231, prostate cancer such as PC3 or LNCAP, liver cancer HepG2 and Huh7, etc.), are injected subcutaneously, intraperitoneally, orthotopically or intravenously (for example, 1×10⁵-1×10⁷ cells) into SCID/SCID, NOD-SCID, NSG, NOG® (Taconic), NCG® (Charles River Laboratories) or CD34 hu-NSG® (Jackson Labs) mice. Subcutaneous, orthotopic or disseminated tumor growth is measured twice a week by Caliper or imaging. Either at the day of tumor cells administration, or when the tumors are established (50-200 mm³), or as seen by appearance of a biomarker, δ1, δ2, or δ3 γδ T-cells (1-100×10⁶) cells, un-engineered or engineered as described herein are adoptively transferred in PBS via tail vein or intraperitoneal injection into the tumor-bearing mice in the absence or presence of cytokines such as IL-2, IL-15, or IL-7. Animals in each group are segregated into treatment groups (treated with different activating agents) or a vehicle control group. At a pre-defined time point before or after the γδ T-cell adoptive transfer (e.g., at day −1, 0, 1, 2, 3 or later) animals are administered vehicle control or δ1, or δ2 or δ3 specific activating agent. The effect of the activating agents in combination with aminobisphosphonates (at a same or different dose and/or dosing schedule) is also tested. The activating agents are administered once or twice a week based on the agent half-life in the mice and potency, in 1 or more cycles until the study termination. The activating agents can be given at a 0.001-1 mg dose per animal 1-4 times a week.

Using CD34+ hu-NSG® or equivalent mice humanized by engraftment CD34+ progenitor cells at birth, illustrates the effect of the activating agents on the endogenous γδ T-cell populations that develop in such model animals in the presence or absence of human xenograft tumors.

### Example 2. Expansion of γδ T Cell Populations In Vivo

Activating agents are used to activate and expand different γδ T-cell populations of a subject's endogenous γδ T-cells and/or γδ T-cells administered to the subject, e.g., after ex-vivo expansion, as un-engineered or engineered cells.

For in vivo expansion γδ1 T-cell populations, subjects receive one or more δ1 γδ T-cell activating agents. For in vivo expansion γδ2 T-cell populations, patients receive one or more δ2 γδ T cell activating agents, and for in vivo expansion γδ3 T-cell populations, patients receive one or more δ3 γδ-cell activating agents. Activating agents, such as MAbs, are formulated to be administered via any route capable of delivering the antibodies to the blood, tumor tissues and other tissues, where γδ T-cell populations reside. Routes of administration include, but are not limited to, intravenous, intraperitoneal, intramuscular, intratumor, intradermal, and the like. Treatment generally involves repeated administration of the activating MAb preparation, via an acceptable route of administration, typically at a dose in the range, including but not limited to, 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, or 25 mg/kg body weight. In general, doses in the range of 10-1000 mg MAb per week are effective and well tolerated. Preferably, the initial loading dose is administered as a 90-minute or longer infusion. The periodic maintenance dose is administered as a 30 minute or longer infusion, provided the initial dose was well tolerated. As appreciated by those of skill in the art, various factors can influence the ideal dose regimen in a particular case. Such factors include, for example, the binding affinity and half-life of the MAbs used, the number of circulating or target-tissue resident γδ T cells in the subject, MAb isotype, the desired steady-state antibody concentration level, frequency of treatment, and the influence of chemotherapeutic or other agents used in combination with the in vivo expansion method of the invention, as well as the health status of a particular patient.

An initial loading dose of approximately 4 mg/kg patient body weight IV, followed by weekly doses of about 2 mg/kg IV of the MAb preparation represents an exemplary dosing regimen. The one or more activating agents are administered weekly, bi-weekly or monthly based on the considerations listed above, for one or more cycles of treatment. Non-limiting preferred human unit doses are, for example, 500 μg-1 mg, 1 mg-50 mg, 50 mg-100 mg, 100 mg-200 mg, 200 mg-300 mg, 400 mg-500 mg, 500 mg-600 mg, 600 mg-700 mg, 700 mg-800 mg, 800 mg-900 mg, 900 mg-1 g, or 1 mg-700 mg. In certain embodiments, the dose is in a range of 2-5 mg/kg body weight, e.g., with follow on weekly doses of 1-3 mg/kg; 0.5 mg, 1, 2, 3, 4, 5, 6, 7, 8, 9, 10 mg/kg body weight followed, e.g., in two, three or four weeks by weekly doses; 0.5-10 mg/kg body weight, e.g., followed in two, three or four weeks by weekly doses; 225, 250, 275, 300, 325, 350, 375, 400 mg m² of body area weekly; 1-600 mg m² of body area weekly; 225-400 mg m² of body area weekly; these does can be followed by weekly doses for 2, 3, 4, 5, 6, 7, 8, 9, 19, 11, 12 or more weeks.

The dose and administration frequency is selected to support expansion or maintenance of the relevant γδ T-cell populations. The effect on the frequency, phenotype, and activation status of the patient's circulating γδ T-cells is tested on 1, 2, 3, 7, 14 or 28 days after the activating agent administration. In addition, the effect on the frequency, phenotype, and activation state of αβ T-cells, NK cells, and/or dendritic cells can be tested.

In some treatment regimens, the activating agents are administered together with of IL-2 or other cytokines, including but not limited to IL-15. When activating agents are administered with aminobisphosphonates, the aminobisphosphonates, such as such pamidronate or zoledronic acid) are administered prior, in conjunction, or after the activating agent administration.

The therapeutic effect of the activating agents is evaluated based the clinical response. For example, for treatment of solid tumors, the Recist criteria can be used. The responses are measured at 1, 3, 6, 9 and 12 months after the beginning of the treatment. Vital signs, electrocardiograms, clinical laboratory test results, and adverse events are used to assess safety. Tumor assessments, such as computed tomography (CT) scans, are performed at baseline and every 8 weeks while subjects are on the study. Serial blood samples are collected from each subject for presence of infused cells. All statistical tests used for the analysis of efficacy and safety data are two-sided and performed at 0.05 level of significance and the 90% confidence interval is computed.

The described studies are performed in a multi-center study and conducted with subjects having locally advanced and/or metastatic hematological or solid cancers that have been previously treated or for whom there is no effective standard treatment available. Un-engineered or engineered γδ T cells (1×10⁸, 2×10⁸, 5×10⁸, and 1×10⁹-one cohort at each dose level) are administered intravenously over 4 hours once, or every four weeks on day 1 of each cycle

### Example 3. Expansion of γδ T Cell Populations In Vivo

NOG non-tumor bearing mice that express an hIL-15 transgene are obtained from Taconic (NOD.Cg-Prkdc^{scid} IL2rg^{tm1Sug} Tg(CMV-IL2/IL15)1-1Jic/JcTac) were inoculated with Vδ1, Vδ2, or Vδ3 γδ CAR-T cells expanded as described in WO 2017/197347 or WO 2019/099744. Cells were cryopreserved after expansion and thawed one day prior to administration into cell culture media containing IL-2. On the day of administration, cells were labeled with CellTrace Violate for 30 minutes as per manufacturer instructions and dosed iv at 20×10⁶ cells/animal. A subset of animals also received ip injection of non-specific murine IgG fraction 4-5 hrs prior to administration of cells.

On Day 2, post cell administration, animals were administered Vδ-subtype specific activating agents as indicated. Control animals were not administered activating agents. On Day 4 or 5, animals were bled and circulating human γδ T cells were characterized for identify, activation markers, and proliferation. On Day 7 post cell administration, animals were sacrificed and various organs were harvested. Spleen and bone marrow were disintegrated using a GentleMax (Miltenyi) device and single-cell suspensions were analyzed by flow cytometry.

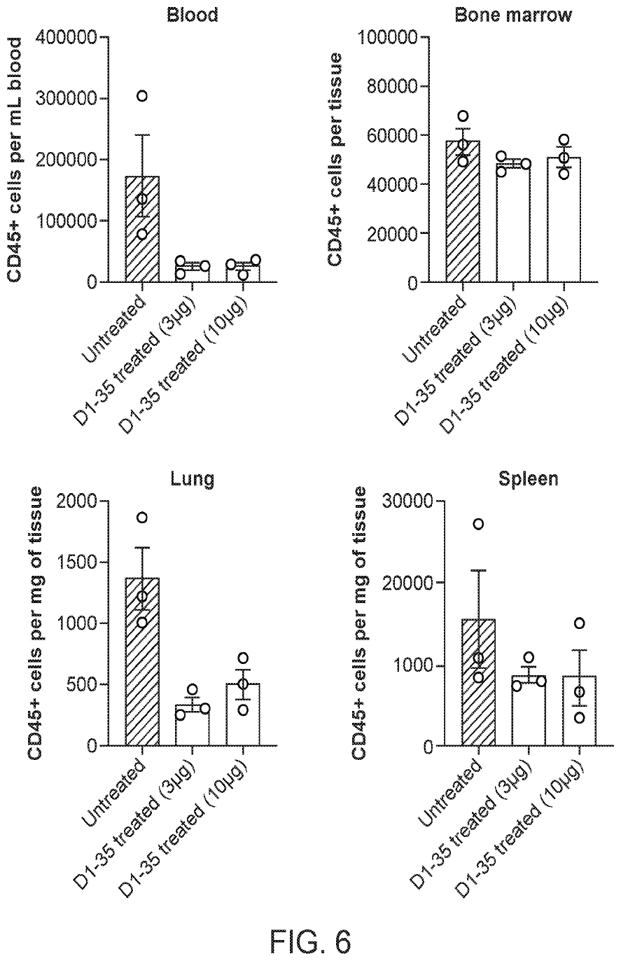

As shown in FIG. 6, the detected number of total human CD45⁺ cells in circulation and in the lung decreased compared to control untreated animals at both doses of activating agent (3 and 10 μg per animal). The number of cells found in other tissues tested (bone marrow, spleen) did not experience significant reduction.

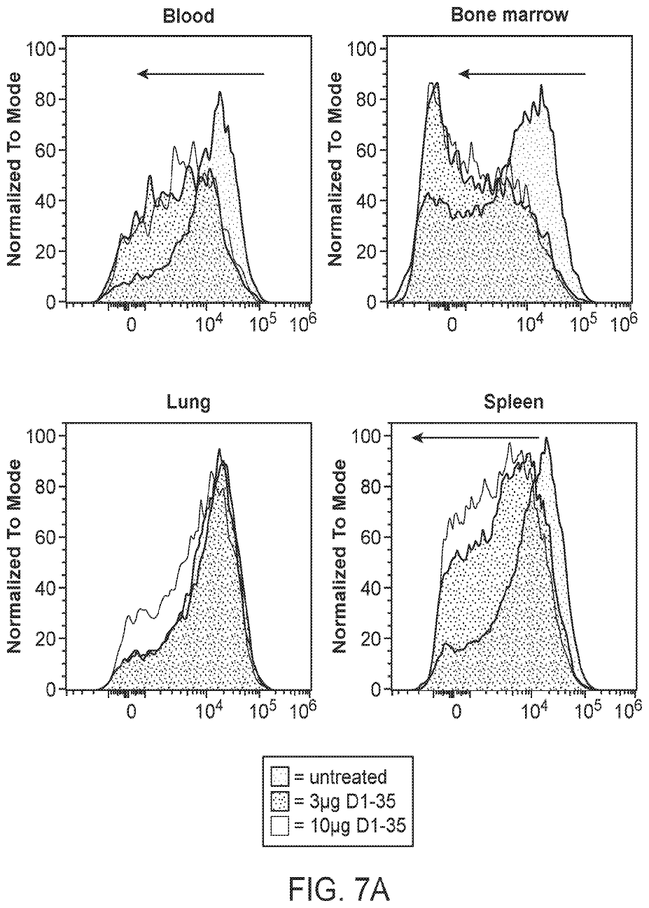

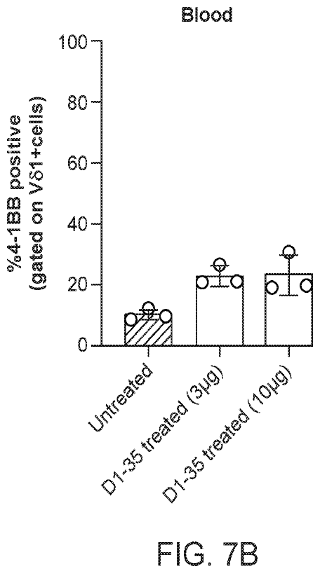

As shown in FIG. 7, cells in blood, bone-marrow and spleen have proliferated by Day 5 post treatment with activating agent, as evidenced by a shift in the CellTrace Violet profiles to the left toward decreased fluorescent intensity due to dye dilution in cellular progeny at both dose levels. CD137 upregulation in circulating cells treated with D1-35 MAb activating agent was also detected, providing further indication of activation.

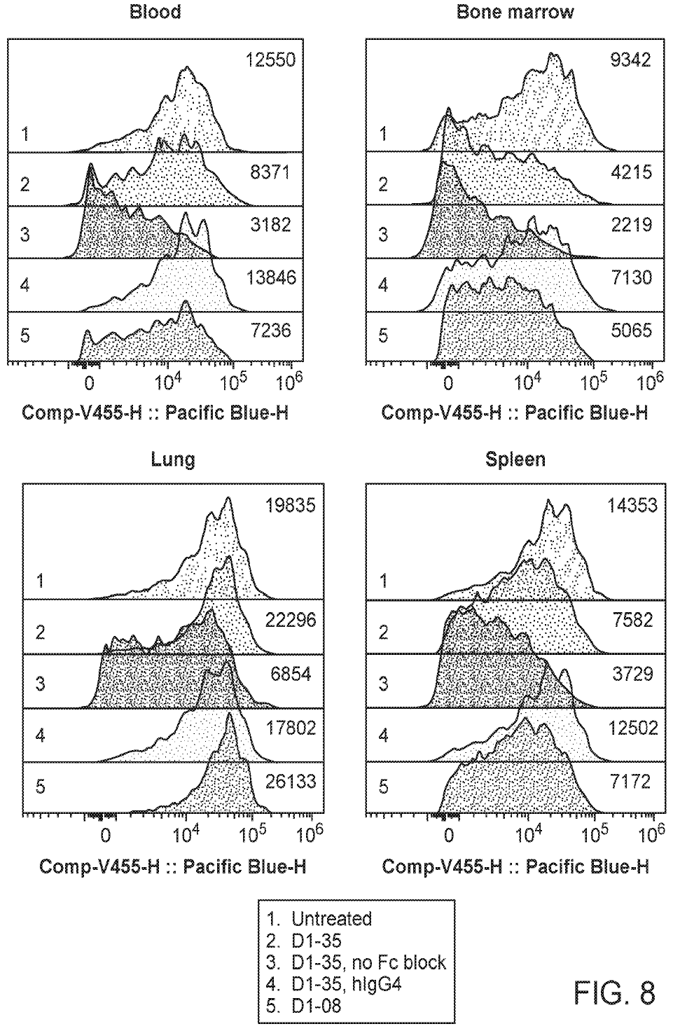

As shown in FIG. 8, animals that were administered D1-35 activating MAb but were not administered IgG fraction (row 3), exhibited increased activation as compared to animals administered both IgG fraction and D1-35 (row 2). Similarly, activating agent D1-08 MAb (row 5), a WI-specific antibody that binds an epitope distinct from D1-35, also has activating properties in vivo and induces cell proliferation as detected by CellTrace Violet. In contrast, changing the isotype of D1-35 antibody to hIgG4 significantly impaired the ability of the MAb to induce cell proliferation (row 4). hIgG4 exhibits significantly reduced affinity for Fc receptor. Accordingly, the impaired ability to induce cell proliferation may be due to reduced immobilization to Fc receptor. Without wishing to be bound by theory, the present inventors hypothesize that immobilization on the surface of a cell expressing an Fc receptor increases the activation effect provided by the administered antibodies on the administered γδ T cells, presumably by increasing TCR aggregation by cross-linking with the immobilized antibody. The present inventors further hypothesize that this increased TCR aggregation can also be achieved using an antigen-binding moiety with a high valency, e.g., a valency higher than IgG. Accordingly, one object of the present invention is, e.g., monospecific, highly multivalent γδ T cell activating agents. For example, robust γδ T cell activation, expansion, and/or maintenance can be obtained by contacting the γδ T cell with an, e.g., trivalent, tetravalent, pentavalent, etc., activating agent. In some cases, the contacting is performed in vivo (e.g., by administering the activating agent to a subject).

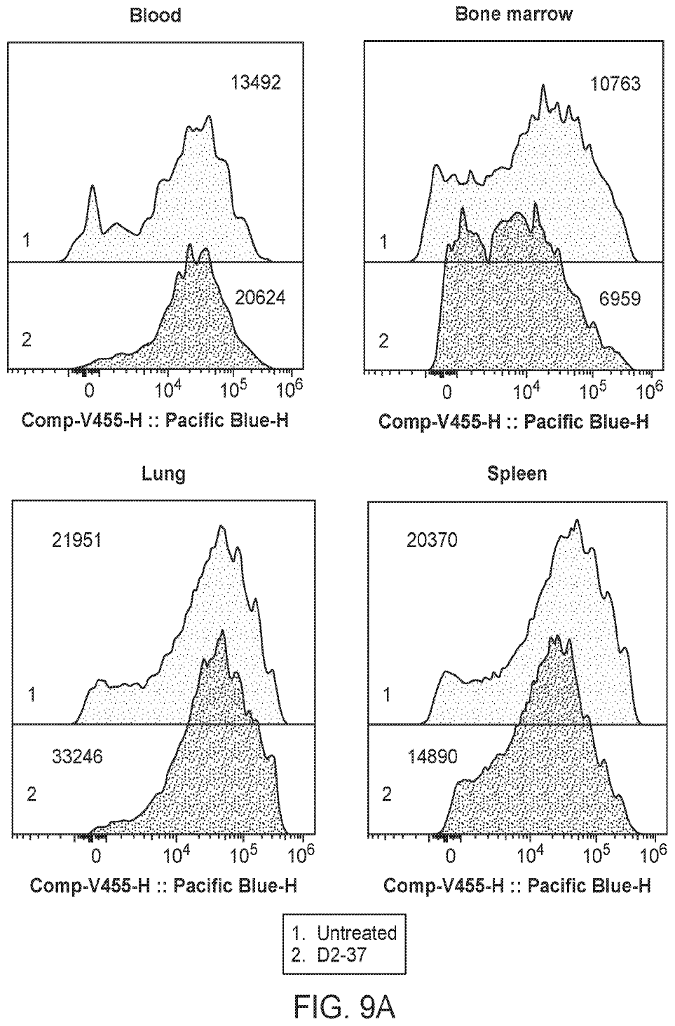

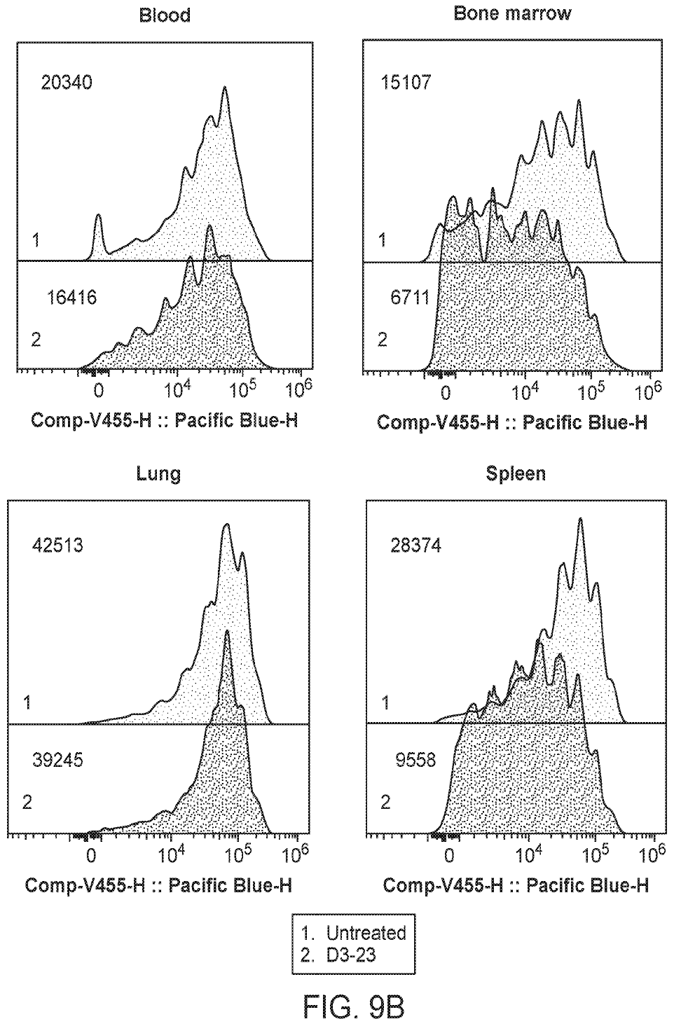

As shown in FIGS. 9A-B, proliferation of Vδ2 and Vδ3 cells was detected in bone marrow and spleen of animals treated with D2-37 and D3-23 MAb respectively as detected by CellTerace Violet dye dilution.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An in vivo method for activating, expanding, and/or maintaining a population of γδ T cells in a subject, the method comprising administering to the subject an effective amount of one or more agents which selectively expand δ1 T cells, δ2 T cells, or δ3 T cells, or a combination thereof, wherein the one or more agents that selectively expand δ1 T cells bind to an activating epitope specific of a δ1 TCR;
the one or more agents that selectively expand δ2 T cells bind to an activating epitope specific of a δ2 TCR; and
the one or more agents that selectively expand δ3 T cells bind to an activating epitope specific of a δ3 TCR,
thereby activating, expanding and/or maintaining the population of γδ T cells in the subject.

2. The method of claim 1, wherein the method comprises administering to the subject an effective amount of one or more agents that selectively expand δ1 T cells.

3. The method of claim 2, wherein the agent that selectively expands δ1 T-cells is selected from an agent which binds to the same epitope as an antibody selected from TS-1 and TS8.2.

4. The method of claim 2, wherein the agent that selectively expands δ1 T-cells is selected from an agent that does not compete with TS-1, TS8.2, or R9.12.

5. The method of claim 2, wherein the agent that selectively expands 81 T-cells is selected from an agent which specifically binds to an epitope comprising a 81 variable region.

6. The method of claim 2, wherein the agent that selectively expands 81 T-cells binds to an epitope comprising residues Arg71, Asp72 and Lys120 of the M variable region.

7. The method of claim 2, wherein the agent that selectively expands 81 T-cells has reduced binding to a mutant M TCR polypeptide comprising a mutation at K120 of delta J1 and delta J2.

8. The method of claim 2, wherein the agents that selectively expand δ1 T-cells are agents that bind a δ1 TCR Bin 1 δ1 epitope, Bin 1b δ1 epitope, Bin 2 δ1 epitope, Bin 2b δ1 epitope, Bin 2c δ1 epitope, Bin 3 δ1 epitope, Bin 4 δ1 epitope, Bin 5 δ1 epitope, Bin 6 δ1 epitope, Bin 7 δ1 epitope, Bin 8 δ1 epitope, or a Bin 9 δ1 epitope of a human δ1 TCR.

9. The method of claim 2, wherein the agent that selectively expands δ1 T-cells is selected from an agent which binds to the same, or essentially the same, epitope as, or competes with, an antibody selected from the group consisting of 61-05, δ1-08, δ1-18, 81-22, δ1-23, δ1-26, δ1-35, δ1-37, δ1-39, δ1-113, δ1-143, δ1-149, δ1-155, δ1-182, δ1-183, δ1-191, δ1-192, δ1-195, δ1-197, δ1-199, δ1-201, 61-203, δ1-239, δ1-253, δ1-257, δ1-278, δ1-282, and δ1-285.

10. The method of claim 2, wherein the agent that selectively expands δ1 T-cells is an antibody selected from the group consisting of 61-05, δ1-08, δ1-18, 81-22, δ1-23, δ1-26, δ1-35, δ1-37, 61-39, δ1-113, δ1-143, δ1-149, δ1-155, δ1-182, δ1-183, δ1-191, δ1-192, δ1-195, δ1-197, δ1-199, δ1-201, 61-203, δ1-239, δ1-253, δ1-257, δ1-278, δ1-282, and δ1-285.

11. The method of claim 2, wherein the agent that selectively expands δ1 T-cells selectively expands δ1 T cells and δ3 T cells.

12. The method of claim 2, wherein the agent that selectively expands M T cells comprises the CDRs of antibody M-35 or δ1-08, or binds the same epitope as antibody δ1-08 or δ1-35.

13. The method of claim 2, wherein the agent that selectively expands M T cells comprises the CDRs of antibody δ1-35.

14. The method of claim 2, wherein the agent that selectively expands δ1 T cells selectively expands δ1, δ3, δ4, and δ5 γδ T cells.

15. The method of claim 1, wherein the method comprises administering to the subject an effective amount of one or more agents that selectively expand δ2 T cells.

16. The method of claim 15, wherein the agent that selectively expands δ2 T-cells is selected from an agent which binds to the same epitope as an antibody selected from 15D and B6.

17. The method of claim 15, wherein the agent that selectively expands δ2 T-cells is selected from an agent which specifically binds to an epitope comprising a δ2 variable region.

18. The method of claim 15, wherein the agent that selectively expands δ2 T-cells has reduced binding to a mutant δ2 TCR polypeptide comprising a mutation at G35 of the δ2 variable region.

19. The method of claim 15, wherein the agent that selectively expands δ2 T-cells binds a δ2 TCR Bin 1 δ2 epitope, Bin 2 δ2 epitope, Bin 3 δ2 epitope, or Bin 4 δ2 epitope of a human δ2 TCR.

20. The method of claim 15, wherein the agent that selectively expands δ2 T-cells is an agent that binds to the same, or essentially the same, epitope as, or competes with, an antibody selected from the group consisting of δ2-14, δ2-17, δ2-22, δ2-30, δ2-31, δ2-32, δ2-33, δ2-35, δ2-36, and δ2-37.

21. The method of claim 15, wherein the agent that selectively expands 82 T-cells is an antibody selected from the group consisting of δ2-14, δ2-17, δ2-22, δ2-30, δ2-31, δ2-32, δ2-33, δ2-35, δ2-36, and δ2-37.

22. The method of claim 15, wherein the agent that selectively expands δ2 T-cells comprises the CDRs of antibody δ2-37 or binds the same epitope as antibody δ2-37.

23. The method of claim 1, wherein the method comprises administering to the subject an effective amount of one or more agents that selectively expand δ3 T cells.

24. The method of claim 23, wherein the agent that selectively expands δ3 T-cells is selected from an agent which binds to the same, or essentially the same, epitope as, or competes with, an antibody selected from the group consisting of δ3-08, δ3-20, δ3-23, δ3-31, δ3-42, δ3-47 and δ3-58.

25. The method of claim 23, wherein the agent that selectively expands 83 T-cells is an antibody selected from the group consisting of δ3-08, δ3-20, δ3-23, δ3-31, δ3-42, δ3-47 and δ3-58.

26. The method of claim 23, wherein the agent that selectively expands δ3 T-cells comprises the CDRs of antibody δ3-23 or binds the same epitope as antibody δ3-23.

27. The method of claim 1, wherein the method comprises administering to the patient a population of engineered and/or non-engineered γδ T cells.

28. The method of claim 27, wherein the method comprises administering the population of engineered and/or non-engineered γδ T cells before administering the one or more agents which selectively expand δ1 T cells, δ2 T cells, or δ3 T cells.

29. The method of claim 27, wherein the administered population of engineered and/or non-engineered γδ T cells is a population of cells that are autologous to the subject.

30. The method of claim 27, wherein the administered population of engineered and/or non-engineered γδ T cells is a population of cells that are allogeneic to the subject.

31. The method of claim 27, wherein the administered population of engineered and/or non-engineered γδ T cells is a population comprising at least 60% δ1 γδ T cells, and the method comprises administering the γδ T cells and sequentially or simultaneously administering the one or more agents which selectively expand δ1 T cells wherein the administered population of engineered and/or non-engineered γδ T cells is a population comprising at least 60% δ2 γδ T cells, and the method comprises administering the γδ T cells and sequentially or simultaneously administering the one or more agents which selectively expand δ2 T cells; or
wherein the administered population of engineered and/or non-engineered γδ T cells is a population comprising at least 60% δ3 γδ T cells, and the method comprises administering the γδ T cells and sequentially or simultaneously administering the one or more agents which selectively expand δ3 T cells.

32. (canceled)

33. (canceled)

34. The method of claim 1, wherein the method comprises administering an aminophosphonate or a prenyl-phosphate.

35. The method of claim 34, wherein the method comprises administering an aminophosphonate and the aminophosphonate is selected from the group consisting of zoledronate, pamidronic acid, alendronic acid, risedronic acid, ibandronic acid, and incadronic acid, or a salt thereof, and/or a hydrate thereof.

36. The method of claim 1, wherein the agent that selectively expands δ1 T cells, δ2 T cells, or δ3 T cells, or a combination thereof is multivalent, preferably wherein the multivalent agent comprises at least two antigen-binding-sites that specifically bind the same antigen, or wherein the multivalent agent comprises at least two antigen-binding sites that specifically bind the same epitope of the same antigen.

37. The method of claim 36, wherein the agent that selectively expands δ1 T cells, δ2 T cells, or δ3 T cells, or a combination thereof is, or is at least, bivalent, trivalent, or tetravalent.

38. The method of claim 1, wherein the method comprises simultaneously or sequentially administering a cytokine to the subject.

39. The method of claim 38, wherein the method comprises administering engineered γδ T cells to the subject, wherein the engineered γδ T cells comprise a transgene that encodes a secreted cytokine.

40. The method of claim 38, wherein the cytokine is a common gamma chain cytokine, or a cytokine selected from the group consisting of IL-2, IL-15 and IL-4, preferably wherein the cytokine is IL-2, IL-15, and/or IL-4.

41. The method of claim 1, wherein the method comprises administering a lymphodepletion protocol to the subject before administering γδ T cells.

42. The method of claim 1, wherein the method comprises repeatedly administering the one or more agents which selectively expand δ1 T cells, δ2 T cells, and/or δ3 T cells.

43. The method of claim 1, wherein the method comprises expanding or maintaining a population of administered γδ T cells in the subject.

44. The method of claim 1, wherein the method comprises expanding or maintaining a population of endogenous γδ T cells in the subject.

45. The method of claim 1, wherein the method comprises expanding a population of endogenous and/or administered γδ T cells in the subject by at least 10% over the amount of γδ T cells in a subject that has not been administered the one or more agents which selectively expand δ1 T cells, δ2 T cells, and/or δ3 T cells.

46. The method of claim 1, wherein the method comprises maintaining a larger population of endogenous and/or administered γδ T cells in the subject by at least 10% over the number of γδ T cells in a subject that has not been administered the one or more agents which selectively expand δ1 T cells, δ2 T cells, and/or δ3 T cells.

47. A method of treating a cancer, infectious disease, inflammatory disease, or an autoimmune disease in a subject in need thereof by performing the method according to claim 1.

48. A use of an agent that selectively expands δ1 T cells, β2 T cells, or δ3 T cells in the manufacture of a medicament for the in vivo expansion of γδ T cells in a subject in need thereof.

49. The use of claim 48, wherein the in vivo expansion of γδ T cells in a subject comprises treating a cancer, infectious disease, inflammatory disease, or an autoimmune disease in the subject.

50. The method of claim 27, wherein the engineered γδ T cells are engineered to stably express one or more tumor recognition moieties.


SEQUENCE LISTING (87 sequences)

SEQ ID NO: 1 - protein, 117 aa, Artificial Sequence
  source: /note=“Description of Artificial Sequence Synthetic polypeptide”
DVQLQESGPGMVKPSQSLSLTCTVTGYSITGGYDWHWIRHFPGNKLEWMAYISYSGSTDYNPSLKSRISVTHDTSKNLFFLNLTSVTTEDTATYYCAREGGRGFAYWGQGTLVTVSA

SEQ ID NO: 2 - protein, 122 aa, Artificial Sequence
  source: /note=“Description of Artificial Sequence Synthetic polypeptide”
QVQLQQSGAELVRPGASVTLSCKASGYTFTDYEVYWVKQTPVHGLEWIGAIDPETGRTAYNQKFKGKAILTTDKSSSTAYMALRSLTSEDSAVYYCARLKSGRYYGDLFAYWGQGTLVTVSA

SEQ ID NO: 3 - protein, 118 aa, Artificial Sequence
  source: /note=“Description of Artificial Sequence Synthetic polypeptide”
EVQLQQSGPELVKPGDSVKMSCKASGYTFTDYYMDWVKQSHGRSLEWIGYIYPKNVGISYNQKFKGKATLTVDKSSSTAYMELHSLTSEDSAVYYCARSLLWDALDYWGQGTSVTVSS

SEQ ID NO: 4 - protein, 121 aa, Artificial Sequence
  source: /note=“Description of Artificial Sequence Synthetic polypeptide”
QVQLQQSGPQLVKPGASVKLSCKASGYTFTSYDINWVKQRPGQGLEWIGWIYPGDGTTDYNGKFKGKATLTVDTSSSSAYMELHSLTSEDSAVYFCARMDDYDDGGAMDYWGQGTSVTVSS

SEQ ID NO: 5 - protein, 122 aa, Artificial Sequence
  source: /note=“Description of Artificial Sequence Synthetic polypeptide”
SDVQLQESGPGLVKPSQSLSVTCTVTGYSITSGYHWNWIRQFPGNRLEWMGYIHNSGSTNYNSFLKSRISITRDTSKNQFFLQLNSVTTEDTATYYCVAYYSNSREFWYAYWGQGTLVTVSA

SEQ ID NO: 6 - protein, 120 aa, Artificial Sequence
  source: /note=“Description of Artificial Sequence Synthetic polypeptide”
EVQLQQSGTVLARPGSSVKMSCKASGYTFTTYWMHWVKQRPGQGLDWIGAIYPGNSDTNYNQKFRGKAKLTAVTSASTAYMELSSLTNEDSAVYYCTYGYYVDYYAMDYWGQGTSVTVSS

SEQ ID NO: 7 - protein, 119 aa, Artificial Sequence
  source: /note=“Description of Artificial Sequence Synthetic polypeptide”
QVQLQQPGAELVRPGASVKLSCKAPGYTFTSYWMNWVKQRPEQGLEWIGKIDPYDSETHYNQKFKDKAILTVDKSSSTAYMQLSSLTSEDSAVYYCARGGDNYDPFAYWGQGTLVTVSA

SEQ ID NO: 8 - protein, 121 aa, Artificial Sequence
  source: /note=“Description of Artificial Sequence Synthetic polypeptide”
QVQLQQPGADLVRPGTSVKLSCKASGYTFTSYWMHWVQQRPGQGLEWIGVIDPSDSYTNYNQKFKGKATLTVDTSSSTAYMQLSSLTSEDSAVYYCARSDDYDEGYFFDQWGQGTTLTVSA

SEQ ID NO: 9 - protein, 121 aa, Artificial Sequence
  source: /note=“Description of Artificial Sequence Synthetic polypeptide”
EVKLEESGGGLVQPGGSMKLSCAASGFTFSDAWMDWVRQSPEKGLEWVAEIRAEANNHATYYAESVKGRFTISRDDSKSRVFLQMNSLRAEDTGIYYCTGLDYGSIGFAYWGQGTLVTVSA

SEQ ID NO: 10 - protein, 120 aa, Artificial Sequence
  source: /note=“Description of Artificial Sequence Synthetic polypeptide”
QVQLQQPGAEIVRPGASVKLSCKASGYAFTDYWMNWVKQRPGQGLEWIGTIDPSDSYASYNQKFKGKATLTVDTSSNSAYMHLSSLTSEDSAVYFCARESNDVCWYFDVWGAGTTVTVSS

SEQ ID NO: 11 - protein, 117 aa, Artificial Sequence
  source: /note=“Description of Artificial Sequence Synthetic polypeptide”
QVQLQQSGAELVRPGASVKLSCKASDYKFTDSEMYWVKQTPVHGLEWIGAIDPETGITAYNQRFKGKATLTSDKSSSTAYMELRSLTSEDSAVYYCTRAVPPWFAYWGQGTLVTVSA

SEQ ID NO: 12 - protein, 114 aa, Artificial Sequence
  source: /note=“Description of Artificial Sequence Synthetic polypeptide”
EVQLQQSGAELGRPGASVKLSCTTSGFNIKDDYMHWVKQRPEQGLEWIGWIDPENGDTAYASKFQGKATITADTSSNTAYLQLSSLTSEDTAVYYCNYYGFDYWGQGTTLTVSS

SEQ ID NO: 13 - protein, 114 aa, Artificial Sequence
  source: /note=“Description of Artificial Sequence Synthetic polypeptide”
QVQLQQSGAELVRPGASVTLSCKASGYKFIDYEMHWVKQTPVHGLEWVGDLDPGTGVTAYNQKFKGKAILTADKSSSTAYMELRSLTSEDSAVYYCTVWSADFWGQGTSVTVSS

SEQ ID NO: 14 - protein, 114 aa, Artificial Sequence
  source: /note=“Description of Artificial Sequence Synthetic polypeptide”
EVQLQMSGAELVRPGASVKLSCTASGFNIKDDYMYWVKQRPEQGLEWIGWIDPENGDTEYASKFQGKATITADTSSNTAYLQLSSLTSEDTAVYYCTYYAMDYWGQGTSVTVSS

SEQ ID NO: 15 - protein, 114 aa, Artificial Sequence
  source: /note=“Description of Artificial Sequence Synthetic polypeptide”
EVQLQQSGAELVRPGASVKLSCTASDFNIKDDYMHWVKQRPEQGLEWIGWIDPENGETEYASKFQGKATLTADTSSNTAYLQLSSLTSEDTAVYYCTELGFDYWGQGTTLTVSS

SEQ ID NO: 16 - protein, 120 aa, Artificial Sequence
  source: /note=“Description of Artificial Sequence Synthetic polypeptide”
QIQLVQSGPELKKPGETVKISCKVSGDTFTTYGMSWVKQAPGKGLKWMGWINTYSGVPTYADDFKGRFAFSLETSASTAYLQINNLKNEDTATYFCARSSYDYDDAMDYWGQGTSVTVSS

SEQ ID NO: 17 - protein, 121 aa, Artificial Sequence
  source: /note=“Description of Artificial Sequence Synthetic polypeptide”
EVKFEESGGGLVQPGGSMKLSCAASGFTFSDAWMDWVRQSPEKGLEWVAEIRAEANNHATYYAESVKGRFTISRDDSKSRVFLQMNSLRAEDTGIYYCTGLDYGSVGFAYWGQGTLVTVSA

SEQ ID NO: 18 - protein, 118 aa, Artificial Sequence
  source: /note=“Description of Artificial Sequence Synthetic polypeptide”
QVQLQQSGAELVRPGASVTLSCKASGYTFVDYEMHWVKQTPVHGLEWIGAIDPETGITAYNQKFKGKATLTADKSSSTAYMELSSLTSEDSAVYYCIRPRGGSHFDYWGQGTTLTVSS

SEQ ID NO: 19 - protein, 114 aa, Artificial Sequence
  source: /note=“Description of Artificial Sequence Synthetic polypeptide”
EVQLQQSGAELVRPGASVKLSCTASGFNIKDDYMSWVKQRPEQGLEWIGWIDPENGDTEYASKFQGKATITADTSSNTAYLRLSSLTSEDTAVYYCTELGFDYWGQGTTLTVSS

SEQ ID NO: 20 - protein, 118 aa, Artificial Sequence
  source: /note=“Description of Artificial Sequence Synthetic polypeptide”
QVQLQQSGADLVRPGASVTLSCKASGYTFTDYEMHWVKQTPVHGLEWIGAIDPETGITAYNQNFKGKATLTADKSSSTAYMELSSLTSEDSAVYYCTRPRGGSHFDHWGQGTPLTVSS

SEQ ID NO: 21 - protein, 122 aa, Artificial Sequence
  source: /note=“Description of Artificial Sequence Synthetic polypeptide”
SDVQLQESGPGLVKPSQSLSLTCSVTGYSITSGYYWNWIRQFPGNNLEWMGYISHDGSNNYNPALKNRISITRDTSKNQFFLKLNSVTTEDTGTYYCASVYYGDYEVWYTYWGQGTLVTVSA

SEQ ID NO: 22 - protein, 117 aa, Artificial Sequence; identical to SEQ ID NO: 11
  source: /note=“Description of Artificial Sequence Synthetic polypeptide”

SEQ ID NO: 23 - protein, 126 aa, Artificial Sequence
  source: /note=“Description of Artificial Sequence Synthetic polypeptide”
EVQLQQSGPELVKPGASVKMSCKASGYTFTDYYMNWVKQSHGKSLEWIGHINPYNGGTSYNQKFKGKATLTVDKSSSTAYMQLNSLTSEDSAVYYCARNHIYYYDGGYFYYAMDYWGQGTSVTVSS

SEQ ID NO: 24 - protein, 117 aa, Artificial Sequence
  source: /note=“Description of Artificial Sequence Synthetic polypeptide”
QVQLQQSGAELVRPGASVTLSCKASGYRFPDYEMHWVKQTPVHGLEWIGAIDPETGRTAYNQKFRGKAKLTADKSSSTVYMELRSLTSEDSAVYYCTRGYGIQFPYWGQGTLVTVSA

SEQ ID NO: 25 - protein, 117 aa, Artificial Sequence
  source: /note=“Description of Artificial Sequence Synthetic polypeptide”
SDVQLQESGPGLVKPSQSLSLTCSVTGYSITSDYYWNWIRQFPGNKLEWMGYITYDGSNNYNPSLKNRISITRDTSKNQFFLKLNSVTTEDTATYYCARDDGYFDYWGQGTTLTVSS

SEQ ID NO: 26 - protein, 113 aa, Artificial Sequence
  source: /note=“Description of Artificial Sequence Synthetic polypeptide”
EVKLVESGGGLVQPGGSLKLSCATSGFTFSDSYMYWVRQTPEKRLEWVAYISYGGVNTYYPDTVRGRFTISRDNAKSTLYLQMSRLKSEDTAMYYCASRGYYWGQGTSVTVSS

SEQ ID NO: 27 - protein, 118 aa, Artificial Sequence
  source: /note=“Description of Artificial Sequence Synthetic polypeptide”
EVQLVESGGGLVKPGGSLKLSCAASGFTFSDYGMHWVRQAPEKGLEWVAYISSGSRTIYYADTVKGRFTISRDNAKNTLFLQMTSLRSEDTAMYYCAREGAYSSFDYWGQGTTLTVSS

SEQ ID NO: 28 - protein, 111 aa, Artificial Sequence
  source: /note=“Description of Artificial Sequence Synthetic polypeptide”
NIVLTQSPASLAVSLGQRATISCRASESVDGYGNSFMHWYQQKPGQPPKLLIYLASNLESGVPARFSGSGSRTDFTLTIDPVESDDAATYYCQQNNEDPFTFGSGTKLEIK

SEQ ID NO: 29 - protein, 106 aa, Artificial Sequence
  source: /note=“Description of Artificial Sequence Synthetic polypeptide”
QIVLTQSPALMSASPGEKVTMTCSASSSVSYMYWYQQKPRSSPKPWIYFTSNLASGVPARFSGSGSGTSYSLTISSMEAEDAATYYCQQWSSNPLTFGAGTKLELK

SEQ ID NO: 30 - protein, 107 aa, Artificial Sequence
  source: /note=“Description of Artificial Sequence Synthetic polypeptide”
DIQMTQSPASLSVSVGETVTITCRASENIYSNLAWYQQKQGKSPQLLVYAATYLADGVPSRFSGSGSGTQYSLKINSLQSEDFGSYYCQHFWGIPYTFGGGTKLEIK

SEQ ID NO: 31 - protein, 112 aa, Artificial Sequence
  source: /note=“Description of Artificial Sequence Synthetic polypeptide”
DVVMTQTPLTLSVTVGQPASISCKSSQSLLHSNGKTYLNWLLQRPGQSPKLLIYLVSKLESGVPDRFSGSGSGTDFKLKISRVEAEDLGVYYCLQATHFPLTCGAGTKLELK

SEQ ID NO: 32 - protein, 113 aa, Artificial Sequence
  source: /note=“Description of Artificial Sequence Synthetic polypeptide”
DIVMSQSPSSLAVSVGEKVTMSCKSSQSLLYSSNQKNSLAWYQQKPGQSPKLLIYWASTRESGVPDRFTGSGSGTDFTLTISSVKAEDLAVYYCQQDYSYPLTFGGGTKLELK

SEQ ID NO: 33 - protein, 107 aa, Artificial Sequence
  source: /note=“Description of Artificial Sequence Synthetic polypeptide”
DIVMTQSHKFMSTSVGDRVSITCKASQDVSIDVAWYQQKPGQSPKLLIYSASYRYTGVPDRFTGSGSGTDFTFTISNVQAEDLAVYYCQQHYSIPCTFGSGTKLEIK

SEQ ID NO: 34 - protein, 107 aa, Artificial Sequence
  source: /note=“Description of Artificial Sequence Synthetic polypeptide”
DIQMTQSPASLSVSVGETVTITCRASENIYSNLAWYQQKQGKSPQLLVYGARNLADGVPSRFSGSGSGTQYSLKINSLQSEDFGSYFCQHFWDTTFTFGSGTKVEIK

SEQ ID NO: 35 - protein, 112 aa, Artificial Sequence
  source: /note=“Description of Artificial Sequence Synthetic polypeptide”
DVVMTQTPLTLSVTVGQPASISCKSSQSLLHSNGKTYLNWLLQRPGQSPKLLIYLVSKVESGVPDRFSGSGSGTDFTLKISRVEAEDLGLYYCLQVTHFPLTFGAGTKLELK

SEQ ID NO: 36 - protein, 113 aa, Artificial Sequence
  source: /note=“Description of Artificial Sequence Synthetic polypeptide”
DVLMTQTPLSLPVSLGDQASISCRSSQSVVHRNGNTFLEWYLQKPGQSPKLLIYKVSNRFSGVPDRFSGSGSGTDFTLKISRVEAEDLGLYYCFQGSHVPYTFGGGTKLEIKR

SEQ ID NO: 37 - protein, 106 aa, Artificial Sequence
  source: /note=“Description of Artificial Sequence Synthetic polypeptide”
DIQMTQTTSSLSASLGDRVTISCRASQDISNYLNWYQQKPDGTVKLLIYYTSRLHSGVPSRFSGSGSGTDYSLTISNLEQEDIATYFCQQGNTLRTFGGGTKLEIK

SEQ ID NO: 38 - protein, 107 aa, Artificial Sequence
  source: /note=“Description of Artificial Sequence Synthetic polypeptide”
QIVLTQSPALMSASPGEKVTMTCSASSSVSYMYWYQQKPRSSPKPWIYLTSNLAAGVPARFSGSGSGTSYSLTISSMEAEDAATYYCQQWSGDPPTFGGGTKLEIKR

SEQ ID NO: 39 - protein, 113 aa, Artificial Sequence
  source: /note=“Description of Artificial Sequence Synthetic polypeptide”
DVLMTQTPLSLPVSLGDQASISCRSSQSIVHSDGNTFLQWYLQKPGQSPKLLIYKVSNRFSGVPDRFSGSGSGTDFTLKISRVEAEDLGVYYCFQGSHVPYTFGGGTKLEIKR

SEQ ID NO: 40 - protein, 113 aa, Artificial Sequence
  source: /note=“Description of Artificial Sequence Synthetic polypeptide”
SDVVRPTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 41 - protein, 113 aa, Artificial Sequence
  source: /note=“Description of Artificial Sequence Synthetic polypeptide”
DVVMTQIPVSLPVTLGDQASISCRSSQSLLHSDGNTYLHWYLQKPGQSPKLLIYKVSNRFSGVPDRFSGSGSGTDFTLKISRVEAEDLGVYFCSQFTHVPPWTFGGGTKLEIK

SEQ ID NO: 42 - protein, 113 aa, Artificial Sequence
  source: /note=“Description of Artificial Sequence Synthetic polypeptide”
AVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSHSTHVPYTFGGGTKLEIKR

SEQ ID NO: 43 - protein, 108 aa, Artificial Sequence
  source: /note=“Description of Artificial Sequence Synthetic polypeptide”
DVQITQSPSYLAASPGETITINCRTSKSISKYLAWYQEKPGKTNKLLIYSGSTLQSGIPSRFSGSGSGTDFTLTISSLEPEDFAMYYCQHHNEYPYTFGGGTRLEIKR

SEQ ID NO: 44 - protein, 113 aa, Artificial Sequence
  source: /note=“Description of Artificial Sequence Synthetic polypeptide”
DVLMTQTPLSLPVSLGDQASISCRSSQSVVHRNGNTFLEWYLQKPGQSPKLLIYKVSNRFSGVPDRFSGSGSGTDFTLKISRVEAEDLGVYYCFQGSHVPYTFGGGTKLEIKR

SEQ ID NO: 45 - protein, 106 aa, Artificial Sequence
  source: /note=“Description of Artificial Sequence Synthetic polypeptide”
QIVLSQSPAILSASPGEKVTMTCRASSSVNYMHWYQQKPGSSPKPWIYATSNLASGVPARFSGSGSGTSYSLTISRVEAEDAATYYCQQWSSHQPTFGAGTKLELK

SEQ ID NO: 46 - protein, 113 aa, Artificial Sequence; identical to SEQ ID NO: 42
  source: /note=“Description of Artificial Sequence Synthetic polypeptide”

SEQ ID NO: 47 - protein, 106 aa, Artificial Sequence
  source: /note=“Description of Artificial Sequence Synthetic polypeptide”
QIVLSQSPAILSASPGEKVTMTCRASSSVRYIHWYQQKPGSSPKPWIYATSNLASGVPARFSGSGSGTSYSLTISRVEAEDAATYHCQQWYSDTPTFGAGTKLELK

SEQ ID NO: 48 - protein, 113 aa, Artificial Sequence
  source: /note=“Description of Artificial Sequence Synthetic polypeptide”
DIVMSQSPSSLAVSVGEKVALNCKSSQSLLYSINQKNYLAWYQRNPGQSPKLLIYWASTRESGVPDRFTGSGSGTDFTLTISSVKTEDLAVYYCQQYYSYPWTFGGGTKLEIK

SEQ ID NO: 49 - protein, 107 aa, Artificial Sequence; identical to SEQ ID NO: 38
  source: /note=“Description of Artificial Sequence Synthetic polypeptide”

SEQ ID NO: 50 - protein, 107 aa, Artificial Sequence
  source: /note=“Description of Artificial Sequence Synthetic polypeptide”
NIVLTQSPASLAVSLGQRATISCRPSESVDSYGNSFMHWYQQKPGQPPKLLIYLASNLESGVPARFSGSGSRTDLTLTIDPVEADDAATYYCQQNNEDPWTFGGGTK

SEQ ID NO: 51 - protein, 106 aa, Artificial Sequence
  source: /note=“Description of Artificial Sequence Synthetic polypeptide”
QIVLSQSPAILSASPGEKVTMTCRASSSVSYIHWFQQKPGSSPKFWIYGTSNLASGVPARFTGSGSGTSYSLTISRVEAEDAATYYCQQWSSDSPTFGGGTKLEIK

SEQ ID NO: 52 - protein, 106 aa, Artificial Sequence
  source: /note=“Description of Artificial Sequence Synthetic polypeptide”
QIVLSQSPAILSASPGEKVTMTCRASSSVNYMHWHQQKPGSSPKPRIYGTSNLASGVPARFSGSGSGTSYSLTISRVEAEDAATYYCQQWSSNPPTFGAGTKLELK

SEQ ID NO: 53 - protein, 111 aa, Artificial Sequence
  source: /note=“Description of Artificial Sequence Synthetic polypeptide”
DIVLTQSPPSLAVSLGQRATISCKASQSVDYDGDSYMNWYQQKPGQPPKLLIYGASNLESGIPARFSGSGSGTDFTLNIHPMEEEDAATYYCQQSNEDPWTFGGGTKLEIK

SEQ ID NO: 54 - protein, 111 aa, Artificial Sequence
  source: /note=“Description of Artificial Sequence Synthetic polypeptide”
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPWTFGGGTKLEIK

SEQ ID NO: 55 - protein, 122 aa, Artificial Sequence
  source: /note=“Description of Artificial Sequence Synthetic polypeptide”
EVQLQQSGPELVKPGASVKISCKASGYSFTGYYMNWVKQSPEKSLEWIGEINPSTGGTTYNQKFQAKATLTVDKSSSTAYMQLKSLTSEDSAVYYCSRGEDDGYFPYSMDFWGQGASVTVSS

SEQ ID NO: 56 - protein, 118 aa, Artificial Sequence
  source: /note=“Description of Artificial Sequence Synthetic polypeptide”
SDVQLQESGPGLVTPSQSLSVTCTVTGYSITSGSYWNWIRQFPGNKLEWMGYIHNSGSTTYNPSLKSRISITRDTSKNQFFLQLNSVTTEDTATYYCARSTGPPFTYWGQGTLVTVSA

SEQ ID NO: 57 - protein, 120 aa, Artificial Sequence
  source: /note=“Description of Artificial Sequence Synthetic polypeptide”
QVQLQQSGAELVKPGASVKLSCKTSGYTFTSYWIQWVKQRPGQGLGWIGEIFPGTGTTYYNEKFKGKATLTRDTSSSTAYMQLSSLTSEDSAVYFCARRGVFGNYAMDYWGQGTSVTVSS

SEQ ID NO: 58 - protein, 120 aa, Artificial Sequence
  source: /note=“Description of Artificial Sequence Synthetic polypeptide”
QVQLLQPGAELVKPGASVKLSCKASGYSFTNFWINWVKLRPGQGLEWIGNIFPGSSSPNYNEKFKSKATLTVDISSSTAYMQLSSLTSDGSAVYYCARYGSYGNWYFDVWGAGTTVTVSS

SEQ ID NO: 59 - protein, 120 aa, Artificial Sequence
  source: /note=“Description of Artificial Sequence Synthetic polypeptide”
EVKLVESGGGLVQPGGSLSLSCAASGFTFTDYYMSWVRQPPGKALEWLGFIRNKANGYTTESSASVKGRFTISRDNSQSILYLQMNVLRAEDSATYYCASSKPGWPMDYWGQGTSVTVSS

SEQ ID NO: 60 - protein, 122 aa, Artificial Sequence; identical to SEQ ID NO: 55
  source: /note=“Description of Artificial Sequence Synthetic polypeptide”

SEQ ID NO: 61 - protein, 120 aa, Artificial Sequence
  source: /note=“Description of Artificial Sequence Synthetic polypeptide”
DVKLVESGGGLVKPGGSLKLSCAASGFTFSSYYMSWVRQTPEKRLEWVATISNSGGSTYYPDSVKGRFTISRDNAKNTLYLQISSLNSEDTAVYYCSREYDFDGEFFDYWGQGTTLTVSS

SEQ ID NO: 62 - protein, 120 aa, Artificial Sequence
  source: /note=“Description of Artificial Sequence Synthetic polypeptide”
QVQLLQPGAELVKPGASVKLSCKASGYTFTNFWINWVKQRPGQGLEWIGNIYPGSSSPNYNEKFKFKATLTVDISSSTAYIQLSSLPSDDSAVYYCARYGTFGNWYFDVWGAGTTVTVSS

SEQ ID NO: 63 - protein, 121 aa, Artificial Sequence
  source: /note=“Description of Artificial Sequence Synthetic polypeptide”
QVQLQQPGAELVKPGASVKLSCKASGYTFTNHWISWVKQRPGQGLEWIGNIFPGSSSPNYNEKFKSKATLTVDTSSSTAYMQLSSLTSDASAVYYCTRWGNYGYYYAMDYWGQGTSVTVSS

SEQ ID NO: 64 - protein, 107 aa, Artificial Sequence
  source: /note=“Description of Artificial Sequence Synthetic polypeptide”
DIVLIQSPATLSVTPGDSVSLSCRASQSINNNLHWYQQKSHESPRLLIKYVSQSISGIPSRFSGSGSGTDFTLSINSVETEDFGMYFCQQSNSWPLTFGAGTKLELK

SEQ ID NO: 65 - protein, 110 aa, Artificial Sequence
  source: /note=“Description of Artificial Sequence Synthetic polypeptide”
NIVLTQSPGSLAVSLGQRATISCRASESVDNYGNSFMHWYQQKPGQPPKLLIYLASNLESGVPARFSGSGSRTDFTLTIDPVEADDAATYYCQQNNEDPTFGSGTKLEMK

SEQ ID NO: 66 - protein, 107 aa, Artificial Sequence
  source: /note=“Description of Artificial Sequence Synthetic polypeptide”
DIQMTQSPASLSVSVGETVTITCRASENIYSNLAWYQQKQGKSPQLLVYAATNLAGGVPSRFSGSGSGTQYSLKINSLQSEDFGSYYCQHFWGTPRTFGGGTKLEIK

SEQ ID NO: 67 - protein, 108 aa, Artificial Sequence
  source: /note=“Description of Artificial Sequence Synthetic polypeptide”
DIQMTQSPASLSVSVGETVTITCRASENIYSNLAWYQQKQGKSPQLLVYAATNLADGVPSRFSGSGSGTQYSLKINSLQSEDFGSYYCQHFGDTPYTFGGGTKLEIKR

SEQ ID NO: 68 - protein, 108 aa, Artificial Sequence
  source: /note=“Description of Artificial Sequence Synthetic polypeptide”
DLKMTQSPSSMYASLGERVTITCKASQDINSFLSWFQQIPGKSPKTLIYRANRLVDGVPSRFSGSGSGQDYSLTISSLEYEDMGIYYCLQSDEFPYTIGGGTKLEIKR

SEQ ID NO: 69 - protein, 107 aa, Artificial Sequence
  source: /note=“Description of Artificial Sequence Synthetic polypeptide”
DIQMTQTSSSFSVSLGDRVTITCKASEDIYNRLAWYQQKPGNAPRLLISGATSLEAGVPSRFSGSGSGNDYTLSITSLQTEDVATYYCQQYWYTPWTFGGGTKLEIK

SEQ ID NO: 70 - protein, 107 aa, Artificial Sequence; identical to SEQ ID NO: 64
  source: /note=“Description of Artificial Sequence Synthetic polypeptide”

SEQ ID NO: 71 - protein, 113 aa, Artificial Sequence
  source: /note=“Description of Artificial Sequence Synthetic polypeptide”
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSRHVPYTFGGGTKLEIKR

SEQ ID NO: 72 - protein, 108 aa, Artificial Sequence
  source: /note=“Description of Artificial Sequence Synthetic polypeptide”
DIKMTQSPSSMYASLGERVTITCKASQDINNFLSWFQQIPGKSPKTLIYRANRLVDGVPSRFSGSGSGQDYSLTISSLEYEDMGIYYCLQSDEFPYTIGGGTKLEIKR

SEQ ID NO: 73 - protein, 105 aa, Artificial Sequence
  source: /note=“Description of Artificial Sequence Synthetic polypeptide”
QIVLTQSPAIMSASLGEEITLTCSASSRVNYMHWYQQKSGTSPKLLIYSTSNLASGVPSRFSGSGSGTFYSLTIISVEAEDAADYYCHQWSSYPTFGGGTKLEIK

SEQ ID NO: 74 - protein, 121 aa, Artificial Sequence
  source: /note=“Description of Artificial Sequence Synthetic polypeptide”
EEKLEESGGGLVQPGGSMKLSCVASGFIFSIYWMNWVRQSPEKGLEWVGQIRLKSDNYATHYAESVKGRFTISRDDSKSSVYLQMNNLRAEDTGIYYCMYYGSSYERFAYWGQGTLVTVSA

SEQ ID NO: 75 - protein, 118 aa, Artificial Sequence
  source: /note=“Description of Artificial Sequence Synthetic polypeptide”
QVQLQQSGAELVRPGTSVKMSCKATGYTFSNYWTGWVKQRPGHGLERIGDIYPGGGYTNYNEEFKGKATLTADTSSSTVYMLLSSLTFEDSAIYYCARWGSDYAMDYWGQGTSVTVSS

SEQ ID NO: 76 - protein, 115 aa, Artificial Sequence
  source: /note=“Description of Artificial Sequence Synthetic polypeptide”
EVQLQQSGAELVKPGASVKLSCTASGFNIRDTYMHWVKQRPEQGLEWIGRIDPANGNTKYDPKFRGKATITADTSSNTAYLQLSSLTSEGTAVYYCSEGIYFDYWGQGTTLTVSS

SEQ ID NO: 77 - protein, 113 aa, Artificial Sequence
  source: /note=“Description of Artificial Sequence Synthetic polypeptide”
SDVQLQESGPDLVKPSQSLSLTCTVTGYSITSGYGWHWIRQFPGNKLEWMGYISFSGSNKYNPSLKSRISITRDTSKNQFFLQLNSVTTEDTATYYCANLDYWGQGTTLTVSS

SEQ ID NO: 78 - protein, 121 aa, Artificial Sequence
  source: /note=“Description of Artificial Sequence Synthetic polypeptide”
SDVQLQESGPDLVKPSQSLSLTCTVTGYSITSGYNWHWIRQFPGNKLEWMGYIHYSGNTDYNPSLRSRISITRDTSKNQFFLHLNSVPTEDTATYYCARSGITTDWYFDVWGAGTTVTVSS

SEQ ID NO: 79 - protein, 121 aa, Artificial Sequence
  source: /note=“Description of Artificial Sequence Synthetic polypeptide”
EVQLQQSGAELVRPGASVKLSCTASGFNIKDDYMNWVKQRPEQGLDWIGGIDPANGNTKYAPKFQDKATITADTSSNTAYLQLSSLTSEDTAVFYCARYRDYAVDYWGQGWGQGTSVTVSS

SEQ ID NO: 80 - protein, 119 aa, Artificial Sequence
  source: /note=“Description of Artificial Sequence Synthetic polypeptide”
EVKLVESGGGLVQPGGSLKLSCAASGFTFSSYAMSWVRQTPEKRLEWVAYIRDGGGGTYYPDTVEGRFTISRDNAKNTLYLQMSSLKSEDTAMYYCARHPPMNDWFLYWGQGTLVTVSA

SEQ ID NO: 81 - protein, 108 aa, Artificial Sequence
  source: /note=“Description of Artificial Sequence Synthetic polypeptide”
DIQMIQSPASLSVSVGETVTITCRASENIYSNLAWYQQKQGKSPQLLVYVATKLADGVPSRFSGSGSGTQYSLKINSLQSEDFGSYYCQHFWGTPPWTFGGGTKLEIK

SEQ ID NO: 82 - protein, 108 aa, Artificial Sequence
  source: /note=“Description of Artificial Sequence Synthetic polypeptide”
QIVLTQSPAIMSASPGEKVTMTCSASSSVSSRYLHWYQQKSGASPKFWIYGTSNLASGVPARFSGSGSGTSYSLTISSVEAEDAATYYCQQYHSDPPTFGGGTKLEIK

SEQ ID NO: 83 - protein, 108 aa, Artificial Sequence
  source: /note=“Description of Artificial Sequence Synthetic polypeptide”
DILLTQSPAILSVSPGERVSFSCRASQNIGTIIHWYQQRANGSPRLLIKYASESISGIPSRFSGSGSGTDFTLSINSVESEDIADYYCQQSNSWPYTFGGGTKLEIKR

SEQ ID NO: 84 - protein, 108 aa, Artificial Sequence
  source: /note=“Description of Artificial Sequence Synthetic polypeptide”
DIQMTQRTSSLSASLGDRVTISCSASQDITNYLHWFQQKPDGTVKLLIYYTSTLHSGVPSRFSGSGSGTDYSLTISNLEPEDIATYYCQQYSKLPYTFGGGTKLEITR

SEQ ID NO: 85 - protein, 106 aa, Artificial Sequence
  source: /note=“Description of Artificial Sequence Synthetic polypeptide”
QIVLSQSPAILSASPGEKVTMTCRASSSVNYMHWYQQKPGSSPKPWIYATSNLASGVPARFSGSGSGTSYSLTISKVEAEDAATYYCQQWSSHQPTFGAGTKLELK

SEQ ID NO: 86 - protein, 107 aa, Artificial Sequence
  source: /note=“Description of Artificial Sequence Synthetic polypeptide”
DIRMTQSPSSMYASLGERVTITCKASQDINTYLRWCQQKPGKSPKTLIYGANRLVDGVPSRFSGSGSGQDYSLTISSLEYEDMGIYYCLQYDEFPLTFGAGTKLELK

SEQ ID NO: 87 - protein, 108 aa, Artificial Sequence
  source: /note=“Description of Artificial Sequence Synthetic polypeptide”
DIQMTQSPASLSVSVGETVTITCRASENIYSHLAWYQQKQGKSPQLLVYAATNLADGVPSRFSGSGSGTQYSLKINSLQSEDFGSYYCQHFWGTPYTFGGGTKLEIKR